SEQUENCE LISTING (78 sequences)

SEQ ID NO: 1 - protein, 118 aa, Mus musculus
EVQLQQSGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHWGQGTTLTVSS

SEQ ID NO: 2 - protein, 120 aa, Homo sapiens [sequence omitted]

SEQ ID NO: 3 - protein, 118 aa, synthetic construct
  REGION 1..118: note = Antigen binding construct or fragment thereof
EVQLVESGGGLVQPGGSLRLSCAASGFNIKDTYIHWVRQAPGKGLEWVARIDPANDNTLYASKFQGRATISADTSKNTAYLQMNSLRAEDTAVYYCGRGYGYYVFDHWGQGTLVTVSS

SEQ ID NO: 4 - protein, 118 aa, Mus musculus
QVQLQQSGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHWGQGTTLTVSS

SEQ ID NO: 5 - protein, 115 aa, Homo sapiens
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCATAEYFQHWGQGTLVTVSS

SEQ ID NO: 6 - protein, 118 aa, synthetic construct
  REGION 1..118: note = Antigen binding construct or fragment thereof
QVQLVQSGAEVKKPGATVKISCKVSGFNIKDTYIHWVQQAPGKGLEWMGRIDPANDNTLYASKFQGRVTITADTSTDTAYMELSSLRSEDTAVYYCARGYGYYVFDHWGQGTLVTVSS

SEQ ID NO: 7 - protein, 107 aa, Mus musculus
DVQINQSPSFLAASPGETITINCRTSRSISQYLAWYQEKPGKTNKLLIYSGSTLQSGIPSRFSGSGSGTDFTLTISGLEPEDFAMYYCQQHNENPLTFGAGTKLELK

SEQ ID NO: 8 - protein, 107 aa, Homo sapiens
DIQMTQSPSSLSASVGDRVTITCRASQGISNYLAWYQQKPGKVPKLLIYAASTLQSGVPSRFSGSGSGTDFTLTISSLQPEDVATYYCQKYNSAPLTFGGGTKVEIK

SEQ ID NO: 9 - protein, 107 aa, synthetic construct
  REGION 1..107: note = Antigen binding construct or fragment thereof
DVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIK

SEQ ID NO: 10 - DNA, 768 bp, synthetic construct
  misc_feature 1..768: note = Antigen binding construct or fragment thereof
  source 1..768: mol_type = other DNA
atggagaccgatacactgctgctgtgggtgctgctgctctgggtccctggcagcacaggagacatccagatgacacagagccctagctccctgagcgcttccgtgggagatagggtgaccatcacatgccggacctccaggtccatctcccagtacctggcctggtaccagcagaagcccggcaaggtgcccaagctgctcatctatagcggcagcaccctgcagagcggagtgccttcccggttttccggatccggctccggcacagactttaccctgaccatctccagcctgcagcctgaggatgtcgccacctactactgccaacagcacaacgagaaccccctgaccttcggcggcggaaccaaggtcgagatcaagggaggaggctccggaggaggaggccaagtgcagctggtccaatccggcgccgaagtgaaaaagcccggcgccaccgtgaagatcagctgcaaggtgtccggcttcaacatcaaggacacctatatccactgggtccagcaagcccccggaaaaggcctggagtggatgggacggattgaccccgccaacgacaacacactctatgcctccaagttccagggcagggtgacaatcaccgccgacaccagcaccgacacagcttatatggagctgtcctccctccggtccgaggataccgccgtctactactgcgccaggggctacggctactacgtgtttgaccactggggccagggcaccctggtgacagtgtccagcggaggctgc

SEQ ID NO: 11 - DNA, 768 bp, synthetic construct
  misc_feature 1..768: note = Antigen binding construct or fragment thereof
  source 1..768: mol_type = other DNA
atggagaccgacaccctgctgctctgggtcctcctgctgtgggtgcctggcagcacaggacaggtgcaactggtgcagagcggcgccgaggtcaagaaacctggcgccaccgtgaagatcagctgcaaggtgtccggcttcaacatcaaggacacctacatccactgggtccaacaagcccccggaaagggcctggaatggatgggccggattgaccccgccaacgacaacaccctctatgccagcaagttccagggcagggtcaccatcaccgccgacaccagcaccgacaccgcctacatggagctgagcagcctgcggagcgaagacaccgccgtgtactactgcgccaggggctacggctactacgtcttcgaccattggggacagggcaccctcgtgacagtgtccagcggaggaggatccggcggaggaggagatatccagatgacccagagcccttccagcctgtccgcttccgtgggagatcgggtgaccatcacatgcaggacctccaggtccatctcccagtacctggcctggtaccaacagaagcccggcaaggtgcccaagctgctgatctacagcggcagcacactgcaatccggcgtcccttcccggttttccggatccggatccggcaccgacttcaccctgaccatcagctccctgcaacccgaggacgtggccacctactactgtcagcagcacaacgagaaccccctcacctttggcggcggaaccaaggtcgagatcaagggcggctgc

SEQ ID NO: 12 - protein, 253 aa, synthetic construct
  REGION 1..253: note = Antigen binding construct or fragment thereof
METDTLLLWVLLLWVPGSTGDIQMTQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKSGGGGQVQLVQSGAEVKKPGATVKISCKVSGFNIKDTYIHWVQQAPGKGLEWMGRIDPANDNTLYASKFQGRVTITADTSTDTAYMELSSLRSEDTAVYYCARGYGYYVFDHWGQGTLVTVSSGGC

SEQ ID NO: 13 - protein, 253 aa, synthetic construct
  REGION 1..253: note = Antigen binding construct or fragment thereof
METDTLLLWVLLLWVPGSTGQVQLVQSGAEVKKPGATVKISCKVSGFNIKDTYIHWVQQAPGKGLEWMGRIDPANDNTLYASKFQGRVTITADTSTDTAYMELSSLRSEDTAVYYCARGYGYYVFDHWGQGTLVTVSSSGGGGDIQMTQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKGGC

SEQ ID NO: 14 - protein, 256 aa, synthetic construct
  REGION 1..256: note = Antigen binding construct or fragment thereof
METDTLLLWVLLLWVPGSTGDIQMTQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKGGGSGGGGQVQLVQSGAEVKKPGATVKISCKVSGFNIKDTYIHWVQQAPGKGLEWMGRIDPANDNTLYASKFQGRVTITADTSTDTAYMELSSLRSEDTAVYYCARGYGYYVFDHWGQGTLVTVSSGGC

SEQ ID NO: 15 - protein, 256 aa, synthetic construct
  REGION 1..256: note = Antigen binding construct or fragment thereof
METDTLLLWVLLLWVPGSTGQVQLVQSGAEVKKPGATVKISCKVSGFNIKDTYIHWVQQAPGKGLEWMGRIDPANDNTLYASKFQGRVTITADTSTDTAYMELSSLRSEDTAVYYCARGYGYYVFDHWGQGTLVTVSSGGGSGGGGDIQMTQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKGGC

SEQ ID NO: 16 - protein, 394 aa, synthetic construct
  REGION 1..394: note = Antigen binding construct or fragment thereof
METDTLLLWVLLLWVPGSTGDVQINQSPSFLAASPGETITINCRTSRSISQYLAWYQEKPGKTNKLLIYSGSTLQSGIPSRFSGSGSGTDFTLTISGLEPEDFAMYYCQQHNENPLTFGAGTKLELKGSTSGGGSGGGSGGGGSSEVQLQQSGAELVKPGASVKLSCTASGFNIKDTYIHFVRQRPEQGLEWIGRIDPANDNTLYASKFQGKATITADTSSNTAYMHLSSLTSGDTAVYYCGRGYGYYVFDHWGQGTTLTVSSEPKSCDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 1185 bp, synthetic construct
  misc_feature 1..1185: note = Antigen binding construct or fragment thereof
  source 1..1185: mol_type = other DNA
atggagacagacaccctgctcctgtgggtgctgctcctctgggtccctggatccaccggcgatgtccagatcaaccaaagccccagctttctggctgcctcccctggagagacaatcaccatcaattgccggaccagccggagcatttcccagtacctcgcctggtaccaggaaaagcctggcaagaccaacaagctgctgatctactccggctccacactccagagcggcattccctccaggtttagcggatccggatccggaaccgacttcacactcaccatctccggcctggagcccgaggacttcgccatgtattattgccagcagcacaatgagaaccccctgaccttcggcgctggcaccaagctggagctgaaaggctccaccagcggaggcggatccggaggaggaagcggcggcggaggctcctccgaagtgcagctgcaacagagcggcgccgaactggtgaagcctggagcttccgtgaaactcagctgtaccgccagcggcttcaacatcaaggatacctacatccacttcgtgcggcaaaggcctgagcagggcctggaatggatcggcaggatcgaccccgccaacgacaacaccctctacgcctccaagttccaaggcaaggccacaatcaccgctgatacaagctccaacaccgcctacatgcacctcagctccctgaccagcggagacaccgccgtgtactactgcggacggggatacggctactatgtgttcgaccactggggccaaggcaccacactcaccgtgtcctccgagcccaagtcctgcgacaagacacacacctgtcccccttgtggaggaggatcctccggaggcggctccggcggacagcctagggagccccaggtgtacacactgcccccttccagggacgaactcaccaagaaccaggtgtccctgacctgcctggtgaagggattctaccccagcgacatcgccgtggagtgggagtccaacggccaacccgagaacaattacaagaccaccccccctgtgctcgattccgacggctccttcttcctgtactccaagctcaccgtggacaagtcccggtggcaacagggcaatgtgttctcctgcagcgtcatgcacgaggccctgcataaccactacacccagaaatccctcagcctctcccctggaaaatga

SEQ ID NO: 18 - protein, 394 aa, synthetic construct
  REGION 1..394: note = Antigen binding construct or fragment thereof
METDTLLLWVLLLWVPGSTGEVQLQQSGAELVKPGASVKLSCTASGFNIKDTYIHFVRQRPEQGLEWIGRIDPANDNTLYASKFQGKATITADTSSNTAYMHLSSLTSGDTAVYYCGRGYGYYVFDHWGQGTTLTVSSGSTSGGGSGGGSGGGGSSDVQINQSPSFLAASPGETITINCRTSRSISQYLAWYQEKPGKTNKLLIYSGSTLQSGIPSRFSGSGSGTDFTLTISGLEPEDFAMYYCQQHNENPLTFGAGTKLELKEPKSCDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 1185 bp, synthetic construct
  misc_feature 1..1185: note = Antigen binding construct or fragment thereof
  source 1..1185: mol_type = other DNA
atggagaccgacacactcctgctctgggtgctcctgctgtgggtgcctggcagcacaggagaagtgcagctgcagcagtccggcgccgaactcgtcaaacccggagcctccgtcaaactgtcctgcacagccagcggcttcaacatcaaggacacctacatccatttcgtgcggcaaaggcctgaacagggactcgagtggatcggcaggatcgaccccgccaacgacaataccctctacgcctccaagttccagggaaaggccaccattaccgccgacacatccagcaacaccgcctacatgcacctcagctccctgacatccggcgacaccgccgtgtactactgcggcaggggctacggctactacgtgtttgaccactggggccagggaacaaccctgaccgtgtccagcggctccacctccggaggcggaagcggcggaggatccggaggaggaggctcctccgacgtgcaaatcaaccagtcccctagcttcctggccgctagccctggcgagacaatcacaatcaattgtcggaccagccggtccatctcccagtatctggcctggtaccaggagaagcccggcaagacaaacaagctgctcatctacagcggcagcaccctccaatccggcatcccttcccggtttagcggctccggatccggaaccgactttaccctgaccatcagcggcctggaacccgaggatttcgccatgtactactgccagcagcacaacgagaatcccctgacctttggagccggcacaaagctcgagctgaaggagcccaagagctgcgacaaaacccacacctgtcccccttgcggaggaggatcctccggcggcggaagcggaggacaacccagggagccccaggtctacaccctgcctcctagccgggacgaactgacaaagaaccaggtgtccctgacctgtctcgtcaagggcttctacccttccgacatcgccgtcgagtgggaaagcaacggccagcccgagaacaattacaagaccacaccccccgtcctggacagcgatggcagcttcttcctctactccaagctgaccgtggacaagagccggtggcaacaaggcaacgtgttctcctgcagcgtcatgcatgaggccctgcacaatcactacacccagaagagcctgagcctctcccccggcaagtga

SEQ ID NO: 20 - protein, 394 aa, synthetic construct
  REGION 1..394: note = Antigen binding construct or fragment thereof
METDTLLLWVLLLWVPGSTGDIQMTQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKGSTSGGGSGGGSGGGGSSQVQLVQSGAEVKKPGATVKISCKVSGFNIKDTYIHWVQQAPGKGLEWMGRIDPANDNTLYASKFQGRVTITADTSTDTAYMELSSLRSEDTAVYYCARGYGYYVFDHWGQGTLVTVSSEPKSCDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 1185 bp, synthetic construct
  misc_feature 1..1185: note = Antigen binding construct or fragment thereof
  source 1..1185: mol_type = other DNA
atggagacagacaccctcctgctgtgggtcctgctgctgtgggtgcctggcagcacaggagacatccaaatgacccagtcccctagcagcctcagcgcttccgtcggagacagggtcaccatcacatgcaggacctccaggtccatcagccagtatctggcctggtatcagcagaaacccggcaaggtgcctaagctgctgatctacagcggcagcacactccagagcggagtgcccagccggttttccggaagcggatccggaaccgacttcaccctgaccatttccagcctgcaacctgaagacgtggccacctactactgtcagcagcacaacgagaaccccctcaccttcggcggaggcaccaaagtcgagatcaagggcagcaccagcggaggaggaagcggcggaggctccggaggaggaggctcctcccaagtgcagctcgtccaaagcggcgctgaggtgaaaaagcccggcgccacagtcaaaatctcctgcaaggtcagcggcttcaacatcaaggatacctacatccactgggtgcaacaggcccccggcaaaggactcgaatggatgggccggatcgaccctgctaacgacaacacactctacgcctccaagttccagggcagggtgaccatcaccgccgatacctccaccgacacagcctacatggagctgagcagcctgaggtccgaggacaccgccgtctattactgcgcccggggatacggctactacgtgtttgaccattggggacagggaacactcgtgaccgtgagctccgagcccaagagctgcgacaagacccacacatgtcctccttgcggaggaggcagctccggaggcggatccggcggacaacctagggagccccaggtctataccctgccccccagcagggacgagctgacaaagaaccaggtctccctgacctgcctggtgaaaggattctaccccagcgacatcgctgtcgaatgggagtccaacggccagcccgagaacaactacaagacaaccccccccgtgctggattccgacggcagcttcttcctctactccaagctgaccgtcgacaagtccaggtggcagcagggcaacgtgttttcctgctccgtgatgcatgaggccctgcacaaccactacacccagaagtccctgagcctcagccctggcaagtga

SEQ ID NO: 22 - protein, 394 aa, synthetic construct
  REGION 1..394: note = Antigen binding construct or fragment thereof
METDTLLLWVLLLWVPGSTGQVQLVQSGAEVKKPGATVKISCKVSGFNIKDTYIHWVQQAPGKGLEWMGRIDPANDNTLYASKFQGRVTITADTSTDTAYMELSSLRSEDTAVYYCARGYGYYVFDHWGQGTLVTVSSGSTSGGGSGGGSGGGGSSDIQMTQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKEPKSCDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 1185 bp, synthetic construct
  misc_feature 1..1185: note = Antigen binding construct or fragment thereof
  source 1..1185: mol_type = other DNA
atggagaccgatacactgctgctctgggtgctgctgctgtgggtgcctggaagcaccggacaggtgcaactggtccagtccggcgccgaggtgaaaaagcctggcgccaccgtcaagatctcctgtaaggtgagcggcttcaacatcaaggacacctacatccactgggtgcagcaggctcccggaaagggactggagtggatgggcaggatcgaccctgccaatgacaacaccctctacgccagcaagttccaaggacgggtgaccatcacagccgacacatccaccgacacagcctatatggagctctccagcctgaggtccgaggacaccgccgtgtactactgtgccaggggatacggctattacgtgttcgaccactggggacagggcaccctggtgaccgtgagcagcggaagcaccagcggcggaggcagcggaggcggaagcggcggcggcggatcctccgacattcagatgacccaatccccctccagcctgtccgctagcgtgggagacagggtgacaatcacatgtcggacctccaggtccatcagccaatatctcgcctggtatcagcagaagcccggcaaggtgcccaagctcctgatctacagcggctccaccctccaaagcggagtgccttcccggtttagcggaagcggcagcggcacagactttaccctgacaatcagctccctgcaacctgaggacgtcgccacatactactgccagcagcacaacgagaaccctctcacctttggcggcggcaccaaagtggagatcaaggagcccaaatcctgcgacaagacacacacctgccccccttgtggaggaggcagctccggcggcggcagcggcggacaaccccgggaacctcaggtgtatacactccccccttccagggatgagctgaccaagaaccaagtctccctgacctgtctggtgaaaggcttctacccctccgacatcgctgtcgagtgggagagcaacggccagcccgaaaacaactataagaccaccccccccgtgctcgattccgatggcagcttcttcctgtactccaagctcacagtcgacaagagccggtggcaacagggcaacgtcttctcctgtagcgtcatgcacgaggccctccacaaccactacacccagaagtccctctccctgagccccggaaaatga

SEQ ID NO: 24 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 25 - DNA, 60 bp, synthetic construct
  misc_feature 1..60: note = Antigen binding construct or fragment thereof
  source 1..60: mol_type = other DNA
atggaaaccgacaccctgctgctgtgggtgctgctgctctgggtcccaggctccaccggt

SEQ ID NO: 26 - protein, 20 aa, synthetic construct
  REGION 1..20: note = Antigen binding construct or fragment thereof
METDTLLLWVLLLWVPGSTG

SEQ ID NO: 27 - DNA, 15 bp, synthetic construct
  misc_feature 1..15: note = Antigen binding construct or fragment thereof
  source 1..15: mol_type = other DNA
agtggtggaggaggc

SEQ ID NO: 28 - protein, 5 aa, synthetic construct
  REGION 1..5: note = Antigen binding construct or fragment thereof
SGGGG

SEQ ID NO: 29 - DNA, 24 bp, synthetic construct
  misc_feature 1..24: note = Antigen binding construct or fragment thereof
  source 1..24: mol_type = other DNA
ggcggagggagtggcggaggcggc

SEQ ID NO: 30 - protein, 8 aa, synthetic construct
  REGION 1..8: note = Antigen binding construct or fragment thereof
GGGSGGGG

SEQ ID NO: 31-32 - skipped (2 entries)

SEQ ID NO: 33 - DNA, 60 bp, synthetic construct; identical to SEQ ID NO: 25
  misc_feature 1..60: note = Antigen binding construct or fragment thereof
  source 1..60: mol_type = other DNA

SEQ ID NO: 34 - protein, 20 aa, synthetic construct
  REGION 1..20: note = Antigen binding construct or fragment thereof
METDTLLLWVLLLWVPGSTG

SEQ ID NO: 35 - DNA, 54 bp, synthetic construct
  misc_feature 1..54: note = Antigen binding construct or fragment thereof
  source 1..54: mol_type = other DNA
ggctccacatccggcggaggctctggcggtggatctggcggaggcggctcatcc

SEQ ID NO: 36 - protein, 18 aa, synthetic construct
  REGION 1..18: note = Antigen binding construct or fragment thereof
GSTSGGGSGGGSGGGGSS

SEQ ID NO: 37 - DNA, 393 bp, synthetic construct
  misc_feature 1..393: note = Antigen binding construct or fragment thereof
  source 1..393: mol_type = other DNA
gagcctaagtcctgcgacaagacccacacctgtcccccttgcggcggaggaagcagcggaggcggatccggtggccagcctcgggagcctcaggtgtacaccctgcctccctcccgggacgagctgaccaagaaccaggtgtccctgacctgtctggtcaagggcttctacccttccgatatcgccgtggagtgggagtccaacggccagcctgagaacaactacaagaccacccctcctgtgctggactccgacggctccttcttcctgtactccaagctcacagtggataagtcccggtggcagcagggcaacgtgttctcctgctccgtgatgcacgaggccctgcacaaccactatacccagaagtccctgtccctgtctcctggcaag

SEQ ID NO: 38 - protein, 131 aa, synthetic construct
  REGION 1..131: note = Antigen binding construct or fragment thereof
EPKSCDKTHTCPPCGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - DNA, 321 bp, Mus musculus
  source 1..321: mol_type = other DNA
gatgtccagataaaccagtctccatcttttcttgctgcgtctcctggagaaaccattactataaattgcaggacaagtaggagtattagtcaatatttagcctggtatcaagagaaacctgggaaaactaataagcttcttatctactctggatccactctgcaatctggaattccatcaaggttcagtggcagtggatctggtacagatttcactctcaccatcagtggcctggagcctgaagattttgcaatgtattactgtcaacagcataatgaaaacccgctcacgttcggtgctgggaccaagctggagctgaag

SEQ ID NO: 40 - protein, 107 aa, Mus musculus; identical to SEQ ID NO: 7

SEQ ID NO: 41 - DNA, 321 bp, synthetic construct
  misc_feature 1..321: note = Antigen binding construct or fragment thereof
  source 1..321: mol_type = other DNA
gacgtccagataacccagtctccatcctccctgtctgcatctgtaggagacagagtcaccatcacttgcaggacaagtaggagtattagtcaatatttagcctggtatcagcagaaaccagggaaagttcctaagctcctgatctattctggatccactctgcaatctggagtcccatctcggttcagtggcagtggatctgggacagatttcactctcaccatcagcagcctgcagcctgaagatgttgcaacttattactgtcaacagcataatgaaaacccgctcacgttcggcggagggaccaaggtggagatcaaa

SEQ ID NO: 42 - protein, 107 aa, synthetic construct; identical to SEQ ID NO: 9
  REGION 1..107: note = Antigen binding construct or fragment thereof

SEQ ID NO: 43 - DNA, 354 bp, Mus musculus
  source 1..354: mol_type = other DNA
gaggtccagctgcagcagtctggggcagagcttgtgaagccaggggcctcagtcaagttgtcctgcacagcttctggcttcaacattaaagacacctatatacacttcgtgaggcagaggcctgaacagggcctggagtggattggaaggattgatcctgcgaatgataatactttatatgcctcaaagttccagggcaaggccactataacagcagacacatcatccaacacagcctacatgcacctctgcagcctgacatctggggacactgccgtctattactgtggtagaggttatggttactacgtatttgaccactggggccaaggcaccactctcacagtctcctca

SEQ ID NO: 44 - protein, 118 aa, Mus musculus; identical to SEQ ID NO: 1

SEQ ID NO: 45 - DNA, 354 bp, synthetic construct
  misc_feature 1..354: note = Antigen binding construct or fragment thereof
  source 1..354: mol_type = other DNA
gaagtgcagctggtggaaagcggcggcggcctggtgcagccgggcggcagcctgcgcctgagctgcgcggcgagcggctttaacattaaagatacctatattcattttgtgcgccaggcgccgggcaaaggcctggaatggattggccgcattgatccggcgaacgataacaccctgtatgcgagcaaatttcagggcaaagcgaccattagcgcggataccagcaaaaacaccgcgtatctgcagatgaacagcctgcgcgcgggagataccgcggtgtattattgcggccgcggctatggctattatgtgtttgatcattggggccagggcaccctggtgaccgtgagcagc

SEQ ID NO: 46 - protein, 118 aa, synthetic construct
  REGION 1..118: note = Antigen binding construct or fragment thereof
EVQLVESGGGLVQPGGSLRLSCAASGFNIKDTYIHFVRQAPGKGLEWIGRIDPANDNTLYASKFQGKATISADTSKNTAYLQMNSLRAGDTAVYYCGRGYGYYVFDHWGQGTLVTVSS

SEQ ID NO: 47 - DNA, 354 bp, synthetic construct
  misc_feature 1..354: note = Antigen binding construct or fragment thereof
  source 1..354: mol_type = other DNA
gaagtgcagctggtggaaagcggcggcggcctggtgcagccgggcggcagcctgcgcctgagctgcgcggcgagcggctttaacattaaagatacctatattcattttgtgcgccaggcgccgggcaaaggcctggaatggattggccgcattgatccggcgaacgataacaccctgtatgcgagcaaatttcagggcaaagcgaccattagcgcggataccagcaaaaacaccgcgtatctgcagatgaacagcctgcgcgcggaagataccgcggtgtattattgcggccgcggctatggctattatgtgtttgatcattggggccagggcaccctggtgaccgtgagcagc

SEQ ID NO: 48 - protein, 118 aa, synthetic construct
  REGION 1..118: note = Antigen binding construct or fragment thereof
EVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSS

SEQ ID NO: 49 - DNA, 354 bp, synthetic construct
  misc_feature 1..354: note = Antigen binding construct or fragment thereof
  source 1..354: mol_type = other DNA
caggtgcagctggtgcagagcggcgcggaagtgaaaaaaccgggcgcgaccgtgaaaattagctgcaaagtgagcggctttaacattaaagatacctatattcattttgtgcagcaggcgccgggcaaaggcctggaatggattggccgcattgatccggcgaacgataacaccctgtatgcgagcaaatttcagggcaaagcgaccattaccgcggataccagcaccgataccgcgtatatggaactgagcagcctgcgcagcggagataccgcggtgtattattgcggccgcggctatggctattatgtgtttgatcattggggccagggcaccctggtgaccgtgagcagc

SEQ ID NO: 50 - protein, 118 aa, synthetic construct
  REGION 1..118: note = Antigen binding construct or fragment thereof
QVQLVQSGAEVKKPGATVKISCKVSGFNIKDTYIHFVQQAPGKGLEWIGRIDPANDNTLYASKFQGKATITADTSTDTAYMELSSLRSGDTAVYYCGRGYGYYVFDHWGQGTLVTVSS

SEQ ID NO: 51 - DNA, 354 bp, synthetic construct
  misc_feature 1..354: note = Antigen binding construct or fragment thereof
  source 1..354: mol_type = other DNA
caggtgcagctggtgcagagcggcgcggaagtgaaaaaaccgggcgcgaccgtgaaaattagctgcaaagtgagcggctttaacattaaagatacctatattcattttgtgcagcaggcgccgggcaaaggcctggaatggattggccgcattgatccggcgaacgataacaccctgtatgcgagcaaatttcagggcaaagcgaccattaccgcggataccagcaccgataccgcgtatatggaactgagcagcctgcgcagcgaagataccgcggtgtattattgcggccgcggctatggctattatgtgtttgatcattggggccagggcaccctggtgaccgtgagcagc

SEQ ID NO: 52 - protein, 117 aa, synthetic construct
  REGION 1..117: note = Antigen binding construct or fragment thereof
QVQLVQSGAEVKKPGATVKISCKVSGFNIKDTYIHFVQQAPGKGLEWIGRIDPANDNTLYASKFQGKATITADTSTDTAYMELSSLRSEDTAVYYCGRGYGYYVFDHWGQGTLVTVS

SEQ ID NO: 53 - protein, 23 aa, synthetic construct
  REGION 1..23: note = Antigen binding construct or fragment thereof
EPKSCDKTHTCPPCPAPELLGGP

SEQ ID NO: 54 - protein, 25 aa, synthetic construct
  REGION 1..25: note = Antigen binding construct or fragment thereof
EPKSCDKTHTCPPCPGGGSSGGGSG

SEQ ID NO: 55 - protein, 19 aa, synthetic construct
  REGION 1..19: note = Antigen binding construct or fragment thereof
ERKCCVECPPCPAPPVAGP

SEQ ID NO: 56 - protein, 70 aa, synthetic construct
  REGION 1..70: note = Antigen binding construct or fragment thereof
ELKTPLGDTTHTCPRCPEPKSCDTPPPCPRCPEPKSCDTPPPCPRCPEPKSCDTPPPCPRCPAPELLGGP

SEQ ID NO: 57 - protein, 25 aa, synthetic construct
  REGION 1..25: note = Antigen binding construct or fragment thereof
ELKTPLGDTTHTCPRCPAPELLGGP

SEQ ID NO: 58 - protein, 23 aa, synthetic construct
  REGION 1..23: note = Antigen binding construct or fragment thereof
EPKSCDTPPPCPRCPAPELLGGP

SEQ ID NO: 59 - protein, 20 aa, synthetic construct
  REGION 1..20: note = Antigen binding construct or fragment thereof
ESKYGPPCPSCPAPEFLGGP

SEQ ID NO: 60 - protein, 20 aa, synthetic construct
  REGION 1..20: note = Antigen binding construct or fragment thereof
ESKYGPPCPPCPAPEFLGGP

SEQ ID NO: 61 - DNA, 15 bp, synthetic construct
  misc_feature 1..15: note = Restriction site
  source 1..15: mol_type = other DNA
tctagagccgccacc

SEQ ID NO: 62 - DNA, 15 bp, synthetic construct
  misc_feature 1..15: note = Restriction site
  source 1..15: mol_type = other DNA
tctagagccgccacc

SEQ ID NO: 63-64 - skipped (2 entries)

SEQ ID NO: 65 - DNA, 15 bp, synthetic construct
  misc_feature 1..15: note = Restriction site
  source 1..15: mol_type = other DNA
tctagagccgccacc

SEQ ID NO: 66 - skipped

SEQ ID NO: 67 - DNA, 15 bp, synthetic construct
  misc_feature 1..15: note = Restriction site
  source 1..15: mol_type = other DNA
tctagagccgccacc

SEQ ID NO: 68 - skipped

SEQ ID NO: 69 - DNA, 15 bp, synthetic construct
  misc_feature 1..15: note = Restriction site
  source 1..15: mol_type = other DNA
tctagagccgccacc

SEQ ID NO: 70 - skipped

SEQ ID NO: 71 - DNA, 15 bp, synthetic construct
  misc_feature 1..15: note = Restriction site
  source 1..15: mol_type = other DNA
tctagagccgccacc

SEQ ID NO: 72 - skipped

SEQ ID NO: 73 - DNA, 15 bp, synthetic construct
  misc_feature 1..15: note = Restriction site
  source 1..15: mol_type = other DNA
tctagagccgccacc

SEQ ID NO: 74 - skipped

SEQ ID NO: 75 - DNA, 15 bp, synthetic construct
  misc_feature 1..15: note = Restriction site
  source 1..15: mol_type = other DNA
tctagagccgccacc

SEQ ID NO: 76 - skipped

SEQ ID NO: 77 - DNA, 759 bp, synthetic construct
  misc_feature 1..759: note = Antigen binding construct or fragment thereof
  source 1..759: mol_type = other DNA
atggagaccgatacactgctgctgtgggtgctgctgctctgggtccctggcagcacaggagacatccagatgacacagagccctagctccctgagcgcttccgtgggagatagggtgaccatcacatgccggacctccaggtccatctcccagtacctggcctggtaccagcagaagcccggcaaggtgcccaagctgctcatctatagcggcagcaccctgcagagcggagtgccttcccggttttccggatccggctccggcacagactttaccctgaccatctccagcctgcagcctgaggatgtcgccacctactactgccaacagcacaacgagaaccccctgaccttcggcggcggaaccaaggtcgagatcaagtccggaggaggaggccaagtgcagctggtccaatccggcgccgaagtgaaaaagcccggcgccaccgtgaagatcagctgcaaggtgtccggcttcaacatcaaggacacctatatccactgggtccagcaagcccccggaaaaggcctggagtggatgggacggattgaccccgccaacgacaacacactctatgcctccaagttccagggcagggtgacaatcaccgccgacaccagcaccgacacagcttatatggagctgtcctccctccggtccgaggataccgccgtctactactgcgccaggggctacggctactacgtgtttgaccactggggccagggcaccctggtgacagtgtccagcggaggctgc

SEQ ID NO: 78 - DNA, 759 bp, synthetic construct
  misc_feature 1..759: note = Antigen binding construct or fragment thereof
  source 1..759: mol_type = other DNA
atggagaccgacaccctgctgctctgggtcctcctgctgtgggtgcctggcagcacaggacaggtgcaactggtgcagagcggcgccgaggtcaagaaacctggcgccaccgtgaagatcagctgcaaggtgtccggcttcaacatcaaggacacctacatccactgggtccaacaagcccccggaaagggcctggaatggatgggccggattgaccccgccaacgacaacaccctctatgccagcaagttccagggcagggtcaccatcaccgccgacaccagcaccgacaccgcctacatggagctgagcagcctgcggagcgaagacaccgccgtgtactactgcgccaggggctacggctactacgtcttcgaccattggggacagggcaccctcgtgacagtgtccagctccggcggaggaggagatatccagatgacccagagcccttccagcctgtccgcttccgtgggagatcgggtgaccatcacatgcaggacctccaggtccatctcccagtacctggcctggtaccaacagaagcccggcaaggtgcccaagctgctgatctacagcggcagcacactgcaatccggcgtcccttcccggttttccggatccggatccggcaccgacttcaccctgaccatcagctccctgcaacccgaggacgtggccacctactactgtcagcagcacaacgagaaccccctcacctttggcggcggaaccaaggtcgagatcaagggcggctgc

(19) United States
(12) Patent Application Publication
(10) Pub. No.: US 2026/0015422 A1
(43) Pub. Date: Jan. 15, 2026

(54) ANTIGEN BINDING CONSTRUCTS TO CD8

(71) Applicant: IMAGINAB, INC., Inglewood, CA (US)

(72) Inventors: David T. Ho, Long Beach, CA (US); Tove Olafsen, Reseda, CA (US); Giti Agahi, Los Angeles, CA (US); Christian P. Behrenbruch, Inglewood, CA (US)

(21) Appl. No.: 19/339,053

(22) Filed: Sep. 24, 2025

Related U.S. Application Data
(63) Continuation of application No. 16/745,064, filed on Jan. 16, 2020, now Pat. No. 12,435,141.
Application No. 16/745,064 is a division of application No. 16/417,474, filed on May 20, 2019, now abandoned.
Application No. 16/417,474 is a division of application No. 15/230,085, filed on Aug. 5, 2016, now Pat. No. 10,377,826.
Application No. 15/230,085 is a continuation of application No. 14/202,999, filed on Mar. 10, 2014, now abandoned.
(60) Provisional application No. 61/780,286, filed on Mar. 13, 2013.

(51) Int. Cl. C07K 16/28; A61K 39/00; A61K 47/68; A61K 51/10; G01N 33/566; G01N 33/68

(52) U.S. Cl. CPC C07K 16/2815; A61K 47/6849; A61K 51/1027; G01N 33/566; G01N 33/6872; A61K 2039/505; C07K 2317/24; C07K 2317/31; C07K 2317/35; C07K 2317/56; C07K 2317/565; C07K 2317/622; C07K 2317/624; C07K 2317/626; C07K 2317/92; G01N 2333/70517

(57) ABSTRACT

Antigen binding constructs that bind to CD8, for example antibodies, including antibody fragments (such as scFv, minibodies, and cys-diabodies) that bind to CD8, are described herein. Methods of use are described herein.

### CROSS-REFERENCE TO RELATED APPLICATIONS

The present application is a continuation of U.S. application Ser. No. 16/745,064, filed Jan. 16, 2020, which is a divisional of U.S. application Ser. No. 16/417,474, filed May 20, 2019, now abandoned, which is a divisional of U.S. application Ser. No. 15/230,085, filed Aug. 5, 2016, now U.S. Pat. No. 10,377,826, issued Aug. 13, 2019, which is a continuation of U.S. application Ser. No. 14/202,999, filed Mar. 10, 2014, now abandoned, which claims the benefit of U.S. Provisional Application No. 61/780,286, filed Mar. 13, 2013, each of which is hereby incorporated by reference in its entirety.

### REFERENCE TO SEQUENCE LISTING, TABLE, OR COMPUTER PROGRAM LISTING

The present application is being filed along with a Sequence Listing in electronic format. The Sequence Listing is provided as a file entitled SeqListIGNAB014D2C2.xml, created on Sep. 24, 2025, which is 100,305 bytes in size. The information in the electronic format of the Sequence Listing is incorporated herein by reference in its entirety.

### FIELD

Embodiments described herein relate generally to antigen binding constructs, such as antibodies, including antibody fragments, that bind to CD8, (such as minibodies, cys-diabodies, scFv), as well as methods for their use.

BACKGROUND

CD8 (cluster of differentiation 8) is a transmembrane glycoprotein which is a specific marker for a subclass of T-cells (which includes cytotoxic T-cells). CD8 assembles as either a heterodimer of the CD8 alpha and CD8 beta subunits or a CD8 alpha homodimer. The assembled dimeric CD8 complex acts as a co-receptor together with the T-cell receptor (TCR) to recognize antigen presentation by MHC class I cells. CD8 plays a role in the development of T-cells and activation of mature T-cells. Changes in T-cell localization can reflect the progression of an immune response and can occur over time.

### SUMMARY

Some embodiments provided herein relate to antigen binding constructs, such as antibodies, including antibody fragments, that includes a HCDR1 of a HCDR1 sequence in SEQ ID NO:3 or 6; a HCDR2 of a HCDR2 sequence in SEQ ID NO:3 or 6; a HCDR3 of a HCDR3 sequence in SEQ ID NO:3 or 6; a LCDR1 of a LCDR1 sequence in SEQ ID NO:9; a LCDR2 of a LCDR2 sequence in SEQ ID NO:9; and a LCDR3 of a LCDR3 sequence in SEQ ID NO:9). In some embodiments, the antigen binding construct binds specifically to CD8. In some embodiments, the antigen binding construct includes a detectable marker as described herein. In some embodiments, the antigen binding construct includes a therapeutic agent as described herein.

Some embodiments provided herein relate to a humanized cys-diabody that binds to CD8. The humanized cys-diabody can include a polypeptide that comprises from N-terminus to C-terminus: a single-chain variable fragment (scFv) comprising a variable heavy (V_{H}) domain linked to a variable light (V_{L}) domain; and a C-terminal cysteine.

Some embodiments provided herein relate to a humanized minibody that binds to CD8. The humanized minibody can include a polypeptide that comprises, from N-terminus to C-terminus: a single-chain variable fragment (scFv) comprising a variable heavy (V_{H}) domain linked to a variable light (V_{L}) domain; a hinge-extension domain comprising a human IgG1 hinge region; and a human IgG C_{H}3 sequence.

Some embodiments provided herein relate to a nucleic acid encoding an antigen binding construct as described herein, for example a CD8 antibody or antibody fragment.

Some embodiments provided herein relate to a cell line that produces an antigen binding construct as described herein, for example a CD8 antibody or antibody fragment.

Some embodiments provided herein relate to a kit. The kit can include an antigen binding construct as described herein, for example a CD8 antigen binding fragment. In some embodiments, the kit includes a detectable marker.

Some embodiments provided herein relate to a method of detecting the presence or absence of CD8. The method can comprise applying an antigen binding construct as described herein, for example a CD8 antigen binding construct to a sample. The method can include detecting a binding or an absence of binding of the antigen binding construct to CD8. In some embodiments, the method is performed in vivo. In some embodiments, the method is performed in vitro. In some embodiments, part of the method is performed in vivo, and part of the method is performed in vitro.

Some embodiments provided herein relate to a method of targeting a therapeutic agent to a CD8. The method can include administering to a subject an antigen binding construct as described herein, for example a CD8 antibody or antibody fragment. In some embodiments, the antigen binding construct is conjugated to a therapeutic agent.

### BRIEF DESCRIPTION OF THE DRAWINGS

FIG. 1A illustrates some embodiments of a schematic of a minibody having bivalent binding to CD8.

FIG. 1B illustrates some embodiments of a schematic of a minibody.

FIG. 1C provides an example of a CD8 alpha.

FIG. 2A illustrates some embodiments of an alignment of the murine OKT8 Variable Heavy (V_{H}) region against a human antibody and a humanized V_{H} region (the huOKT8 construct).

FIG. 2B illustrates some embodiments of an alignment of the murine OKT8 Variable Light (V_{L}) region against a humanized V_{L} region and the huOKT8 construct.

FIG. 3A illustrates some embodiments of a schematic of a cys-diabody showing bivalent binding to an antigen.

FIG. 3B illustrates a schematic of a cys-diabody showing bivalent binding to an antigen.

FIG. 4 illustrates some embodiments of a chimeric OKT8 minibody V_{L}-V_{H} sequence.

FIG. 5 illustrates some embodiments of a chimeric OKT8 minibody V_{H}-V_{L} sequence.

FIG. 6 illustrates some embodiments of a humanized OKT8 minibody V_{L}-V_{H} sequence.

FIG. 7 illustrates some embodiments of a humanized OKT8 minibody V_{H}-V_{L} sequence.

FIG. 8 illustrates some embodiments of a humanized OKT8 cys-diabody V_{L}-5-V_{L} sequence.

FIG. 9 illustrates some embodiments of a humanized OKT8 cys-diabody V_{H}-5-V_{L} sequence.

FIG. 10 illustrates some embodiments of a humanized OKT8 cys-diabody V_{L}-8-V_{H} sequence.

FIG. 11 illustrates some embodiments of a humanized OKT8 cys-diabody V_{H}-8-V_{L} sequence.

FIG. 12A depicts some embodiments of sequences for cys-diabodies.

FIG. 12B depicts some embodiments of sequences for minibodies.

FIG. 12C depicts some embodiments of sequences for V_{L}.

FIG. 12D depicts some embodiments of sequences for huV_{L}.

FIG. 12E depicts some embodiments of sequences for V_{H}.

FIG. 12F depicts some embodiments of sequences for huV_{H} (version “a” from Version 1).

FIG. 12G depicts some embodiments of sequences for huV_{H} (version “b” from Version 1).

FIG. 12H depicts some embodiments of sequences for huV_{H} (version “c” from Version 2).

FIG. 12I depicts some embodiments of sequences for huV_{H} (version “c” from Version 2).

FIG. 13 illustrates some embodiments of a vector map for pcDNA™ 3.1/myc-His(-) Versions A, B, C.

FIG. 14 illustrates some embodiments of a method of detecting a presence or absence of a target.

FIG. 15 illustrates a western blot analysis of chimeric and humanized OKT8 minibodies.

FIG. 16 is a graph displaying binding of the IAb_Mb_CD8 variants to purified rhCD8 by ELISA.

FIGS. 17A-17D depict the results from the flow cytometry analysis of the IAb_Mb_CD8 variants.

FIGS. 18A and 18B are depictions of gels of western blots of the humanized OKT8 Minibodies.

FIG. 19 is a graph displaying IAb_Mb_CD8 expression analysis by ELISA.

FIG. 20 is a graph depicting the binding of the IAb_Mb_CD8 variants A and B to rhCD8 by ELISA.

FIG. 21 is a graph depicting the binding of IAb_Mb_CD8 variants C and D to rhCD8 by ELISA.

FIGS. 22A and 22B are graphs displaying the flow cytometry analysis of the IAb_Mb_CD8 variants A and B.

FIGS. 23A and 23B are graphs displaying the flow cytometry analysis of the IAb_Mb_CD8 variants C and D.

FIG. 24 depicts an image of a western blot analysis of IAb_Cys-Dba_CD8 variants.

FIG. 25 is a graph depicting the binding of IAb_Cys-Dba_CD8 variants to rhCD8 by ELISA.

FIGS. 26A and 26B display flow cytometry analysis of the IAb_Cys-Dba CD8 variants.

FIG. 27 is a set of graphs depicting the flow cytometry analysis of the IAb_Cys-Dba_CD8 variants.

### DETAILED DESCRIPTION

Described herein are antigen binding constructs, including antibodies and fragments thereof, such as cys-diabodies and minibodies, that bind to a target molecule, CD8. Such antigen binding constructs can be useful for detecting the presence, localization, and/or quantities of the target molecule (CD8 and/or CD8+ cells, for example, certain classes of T-cells). Such antigen binding constructs can also be useful for targeting therapeutic agents to cells that express the target molecule. In some embodiments, methods are provided for detecting the presence or absence of the target molecule (or “target”) using antigen binding constructs (including antibodies, and constructs such as cys-diabodies and/or minibodies). In some embodiments, methods are provided for using the antigen binding constructs for therapeutic purposes.

### Definitions and Various Embodiments

“Treating” or “treatment” of a condition may refer to preventing the condition, slowing the onset and/or rate of development of the condition, reducing the risk of developing the condition, preventing and/or delaying the development of symptoms associated with the condition, reducing or ending symptoms associated with the condition, generating a complete or partial regression of the condition, or some combination thereof. The term “prevent” does not require the absolute prohibition of the disorder or disease.

A “therapeutically effective amount” or a “therapeutically effective dose” is an amount that produces a desired therapeutic effect in a subject, such as preventing, treating a target condition, delaying the onset of the disorder and/or symptoms, and/or alleviating symptoms associated with the condition. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and/or the route of administration. One skilled in the clinical and pharmacological arts will be able to determine a therapeutically effective amount through routine experimentation, for example by monitoring a subject's response to administration of a compound and adjusting the dosage accordingly, given the present disclosure. For additional guidance, see Remington: The Science and Practice of Pharmacy 21.sup.st Edition, Univ. of Sciences in Philadelphia (USIP), Lippincott Williams & Wilkins, Philadelphia, Pa., 2005.

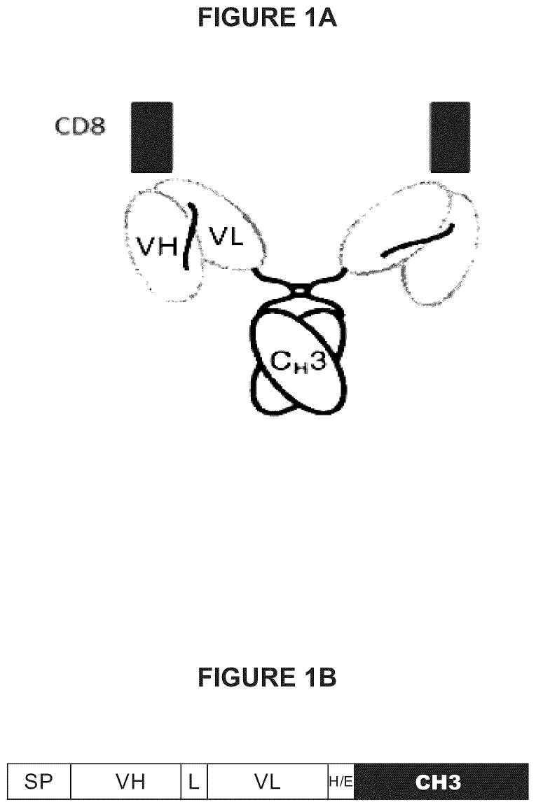

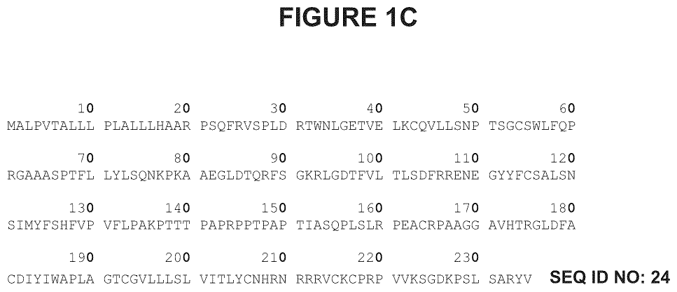

The term “antigen binding construct” includes all varieties of antibodies, including binding fragments thereof. Further included are constructs that include 1, 2, 3, 4, 5, and/or 6 CDRs. In some embodiments, these CDRs can be distributed between their appropriate framework regions in a traditional antibody. In some embodiments, the CDRs can be contained within a heavy and/or light chain variable region. In some embodiments. the CDRs can be within a heavy chain and/or a light chain. In some embodiments, the CDRs can be within a single peptide chain. In some embodiments, the CDRs can be within two or more peptides that are covalently linked together. In some embodiments, they can be covalently linked together by a disulfide bond. In some embodiments, they can be linked via a linking molecule or moiety. In some embodiments, the antigen binding proteins are non-covalent, such as a diabody and a monovalent scFv. Unless otherwise denoted herein, the antigen binding constructs described herein bind to the noted target molecule. The term “target” or “target molecule” denotes the CD8 protein. Examples of CD8 proteins are known in the art, and include, for example the CD8 protein of SEQ ID NO:24, FIG. 1C.

The term “antibody” includes, but is not limited to, genetically engineered or otherwise modified forms of immunoglobulins, such as intrabodies, chimeric antibodies, fully human antibodies, humanized antibodies, antibody fragments, and heteroconjugate antibodies (e.g., bispecific antibodies, diabodies, triabodies, tetrabodies, etc.). The term “antibody” includes cys-diabodies and minibodies. Thus, each and every embodiment provided herein in regard to “antibodies” is also envisioned as cys-diabody and/or minibody embodiments, unless explicitly denoted otherwise. The term “antibody” includes a polypeptide of the immunoglobulin family or a polypeptide comprising fragments of an immunoglobulin that is capable of noncovalently, reversibly, and in a specific manner binding a corresponding antigen. An exemplary antibody structural unit comprises a tetramer. In some embodiments, a full length antibody can be composed of two identical pairs of polypeptide chains, each pair having one “light” and one “heavy” chain (, connected through a disulfide bond. The recognized immunoglobulin genes include the kappa, lambda, alpha, gamma, delta, epsilon, and mu constant region genes, as well as the myriad immunoglobulin variable region genes. For full length chains, the light chains are classified as either kappa or lambda. For full length chains, the heavy chains are classified as gamma, mu, alpha, delta, or epsilon, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (V_{L}) and variable heavy chain (V_{H}) refer to these regions of light and heavy chains respectively. As used in this application, an “antibody” encompasses all variations of antibody and fragments thereof. Thus, within the scope of this concept are full length antibodies, chimeric antibodies, humanized antibodies, single chain antibodies (scFv), Fab, Fab′, and multimeric versions of these fragments (e.g., F(ab′)₂) with the same binding specificity. In some embodiments, the antibody binds specifically to a desired target.

“Complementarity-determining domains” or “complementarity-determining regions (“CDRs”) interchangeably refer to the hypervariable regions of V_{L} and V_{H}. The CDRs are the target protein-binding site of the antibody chains that harbors specificity for such target protein. In some embodiments, there are three CDRs (CDR1-3, numbered sequentially from the N-terminus) in each V_{L} and/or V_{H}, constituting about 15-20% of the variable domains. The CDRs are structurally complementary to the epitope of the target protein and are thus directly responsible for the binding specificity. The remaining stretches of the V_{L} or V_{H}, the so-called framework regions (FRs), exhibit less variation in amino acid sequence (Kuby, Immunology, 4th ed., Chapter 4. W.H. Freeman & Co., New York, 2000).

The positions of the CDRs and framework regions can be determined using various well known definitions in the art, e.g., Kabat (Wu, T. T., E. A. Kabat. 1970. An analysis of the sequences of the variable regions of Bence Jones proteins and myeloma light chains and their implications for antibody complementarity. J. Exp. Med. 132:211-250; Kabat, E. A., Wu, T. T., Perry, H., Gottesman, K., and Foeller, C. (1991) Sequences of Proteins of Immunological Interest, 5th ed., NIH Publication No. 91-3242, Bethesda, MD), Chothia Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997)), ImMunoGeneTics database (IMGT) (on the worldwide web at imgt.org/) Giudicelli, V., Duroux, P., Ginestoux, C., Folch, G., Jabado-Michaloud, J., Chaume, D. and Lefranc, M.-P. IMGT/LIGM-DB, the IMGT® comprehensive database of immunoglobulin and T cell receptor nucleotide sequences Nucl. Acids Res., 34, D781-D784 (2006), PMID: 16381979; Lefranc, M.-P., Pommié, C., Ruiz, M., Giudicelli, V., Foulquier, E., Truong, L., Thouvenin-Contet, V. and Lefranc, G., IMGT unique numbering for immunoglobulin and T cell receptor variable domains and Ig superfamily V-like domains Dev. Comp. Immunol., 27, 55-77 (2003). PMID: 12477501; Brochet, X., Lefranc, M.-P. and Giudicelli, V. IMGT/V-QUEST: the highly customized and integrated system for IG and TR standardized V-J and V-D-J sequence analysis Nucl. Acids Res, 36, W503-508 (2008); AbM (Martin et al., Proc. Natl. Acad. Sci. USA, 86:9268-9272 (1989); the contact definition (MacCallum et al., J. Mol. Biol., 262:732-745 (1996)), and/or the automatic modeling and analysis tool Honegger A, Plückthun A. (world wide web at bioc dot uzh dot ch/antibody/Numbering/index dot html).

The term “binding specificity determinant” or “BSD” interchangeably refer to the minimum contiguous or non-contiguous amino acid sequence within a complementarity determining region necessary for determining the binding specificity of an antibody. A minimum binding specificity determinant can be within one or more CDR sequences. In some embodiments, the minimum binding specificity determinants reside within (i.e., are determined solely by) a portion or the full-length of the CDR3 sequences of the heavy and light chains of the antibody. In some embodiments, CDR3 of the heavy chain variable region is sufficient for the antigen binding construct specificity.

An “antibody variable light chain” or an “antibody variable heavy chain” as used herein refers to a polypeptide comprising the V_{L} or V_{H}, respectively. The endogenous V_{L} is encoded by the gene segments V (variable) and J (junctional), and the endogenous V_{H} by V, D (diversity), and J. Each of V_{L} or V_{H} includes the CDRs as well as the framework regions. In this application, antibody variable light chains and/or antibody variable heavy chains may, from time to time, be collectively referred to as “antibody chains.” These terms encompass antibody chains containing mutations that do not disrupt the basic structure of V_{L} or V_{H}, as one skilled in the art will readily recognize. In some embodiments, full length heavy and/or light chains are contemplated. In some embodiments, only the variable region of the heavy and/or light chains are contemplated as being present.

Antibodies can exist as intact immunoglobulins or as a number of fragments produced by digestion with various peptidases. Thus, for example, pepsin digests an antibody below the disulfide linkages in the hinge region to produce F(ab)′₂, a dimer of Fab′ which itself is a light chain (V_{L}-C_{L}) joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is a Fab with part of the hinge region. (Paul, Fundamental Immunology 3d ed. (1993). While various antibody fragments are defined in terms of the digestion of an intact antibody, one of skill will appreciate that such fragments may be synthesized de novo either chemically or by using recombinant DNA methodology. Thus, the term “antibody,” as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., Monoclonal Antibodies and Cancer Therapy, pp. 77-96. Alan R. Liss, Inc. 1985; Advances in the production of human monoclonal antibodies Shixia Wang, Antibody Technology Journal 2011:1 1-4; J Cell Biochem. 2005 Oct. 1; 96(2):305-13; Recombinant polyclonal antibodies for cancer therapy; Sharon J, Liebman M A, Williams B R; and Drug Discov Today. 2006 July, 11 (13-14): 655-60, Recombinant polyclonal antibodies: the next generation of antibody therapeutics?, Haurum JS). Techniques for the production of single chain antibodies (U.S. Pat. No. 4,946,778) can be adapted to produce antibodies to polypeptides of this invention. Also, transgenic mice, or other organisms such as other mammals, may be used to express fully human monoclonal antibodies. Alternatively, phage display technology can be used to identify high affinity binders to selected antigens (see, e.g., McCafferty et al., supra; Marks et al., Biotechnology, 10:779-783, (1992)).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. In some embodiments, the terms “donor” and “acceptor” sequences can be employed. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some complementarity determining region (“CDR”) residues and possibly some framework (“FR”) residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, and drug; or (b) the variable region, or a portion thereof, is altered, replaced or exchanged with a variable region having a different or altered antigen specificity.

Antibodies further include one or more immunoglobulin chains that are chemically conjugated to, or expressed as, fusion proteins with other proteins. In some embodiments, the antigen binding constructs can be monovalent scFv constructs. In some embodiments, the antigen binding constructs can be bispecific constructs. A bispecific or bifunctional antibody is an artificial hybrid antibody having two different heavy/light chain pairs and two different binding sites. Other antigen-binding fragments or antibody portions of the invention include bivalent scFv (diabody), bispecific scFv antibodies where the antibody molecule recognizes two different epitopes, single binding domains (sdAb or nanobodies), and minibodies.

The term “antibody fragment” includes, but is not limited to one or more antigen binding fragments of antibodies alone or in combination with other molecules, including, but not limited to Fab′, F(ab′)₂, Fab, Fv, rIgG (reduced IgG), scFv fragments (monovalent, tri-valent, etc.), single domain fragments (nanobodies), peptibodies, minibodies, diabodies, and cys-diabodies. The term “scFv” refers to a single chain Fv (“fragment variable”) antibody in which the variable domains of the heavy chain and of the light chain of a traditional two chain antibody have been joined to form one chain.

A pharmaceutically acceptable carrier may be a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or some combination thereof. Each component of the carrier is “pharmaceutically acceptable” in that it is be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. The pharmaceutical compositions described herein may be administered by any suitable route of administration. A route of administration may refer to any administration pathway known in the art, including but not limited to aerosol, enteral, nasal, ophthalmic, oral, parenteral, rectal, transdermal (e.g., topical cream or ointment, patch), or vaginal. “Transdermal” administration may be accomplished using a topical cream or ointment or by means of a transdermal patch. “Parenteral” refers to a route of administration that is generally associated with injection, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. In some embodiments, the antigen binding construct can be delivered intraoperatively as a local administration during an intervention or resection.

The term “CD8 dependent disorder” includes cancers for which there is an immunological component (including response to cancer immunotherapies), autoimmune disorders inflammation disorders, etc.

A minibody is an antibody format that has a smaller molecular weight than the full-length antibody while maintaining the bivalent binding property against an antigen. Because of its smaller size, the minibody has a faster clearance from the system and enhanced penetration when targeting tumor tissue. With the ability for strong targeting combined with rapid clearance, the minibody is advantageous for diagnostic imaging and delivery of cytotoxic/radioactive payloads for which prolonged circulation times may result in adverse patient dosing or dosimetry.

The phrase “specifically (or selectively) bind,” when used in the context of describing the interaction between an antigen, e.g., a protein, to an antibody or antibody-derived binding agent, refers to a binding reaction that is determinative of the presence of the antigen in a heterogenous population of proteins and other biologics, e.g., in a biological sample, e.g., a blood, serum, plasma or tissue sample. Thus, under designated immunoassay conditions, in some embodiments, the antibodies or binding agents with a particular binding specificity bind to a particular antigen at least two times the background and do not substantially bind in a significant amount to other antigens present in the sample. Specific binding to an antibody or binding agent under such conditions may require the antibody or agent to have been selected for its specificity for a particular protein. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity). Typically a specific or selective binding reaction will produce a signal at least twice over the background signal and more typically at least than 10 to 100 times over the background.

The term “equilibrium dissociation constant (K_{D}, M)” refers to the dissociation rate constant (k_{d}, time⁻¹) divided by the association rate constant (kₐ, time⁻¹, M⁻¹). Equilibrium dissociation constants can be measured using any known method in the art. The antibodies of the present invention generally will have an equilibrium dissociation constant of less than about 10⁻⁷ or 10⁻⁸ M, for example, less than about 10⁻⁹ M or 10⁻¹⁰ M, in some embodiments, less than about 10⁻¹¹ M, 10⁻¹² M, or 10⁻¹³ M.

The term “isolated,” when applied to a nucleic acid or protein, denotes that the nucleic acid or protein is essentially free of other cellular components with which it is associated in the natural state. In some embodiments, it can be in either a dry or aqueous solution. Purity and homogeneity can be determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. In some embodiments, this can denote that the nucleic acid or protein is at least 85% pure, more preferably at least 95% pure, and most preferably at least 99% pure of molecules that are present under in vivo conditions.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., an .alpha.-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (sec, e.g., Creighton, Proteins (1984)).

“Percentage of sequence identity” can be determined by comparing two optimally aligned sequences over a comparison window, wherein the portion of the polynucleotide sequence in the comparison window may comprise additions or deletions (i.e., gaps) as compared to the reference sequence (e.g., a polypeptide of the invention), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

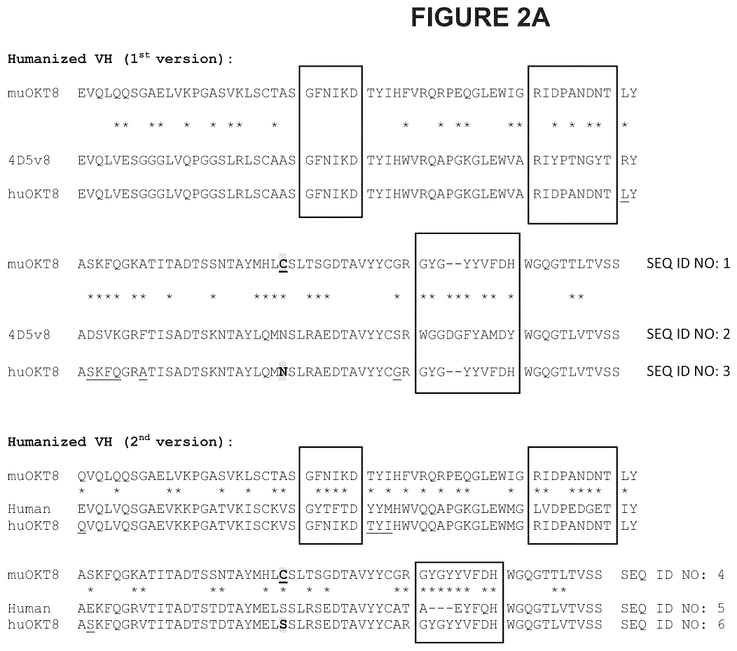

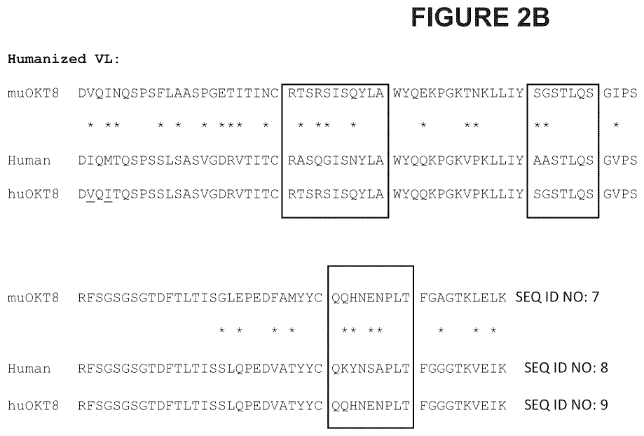

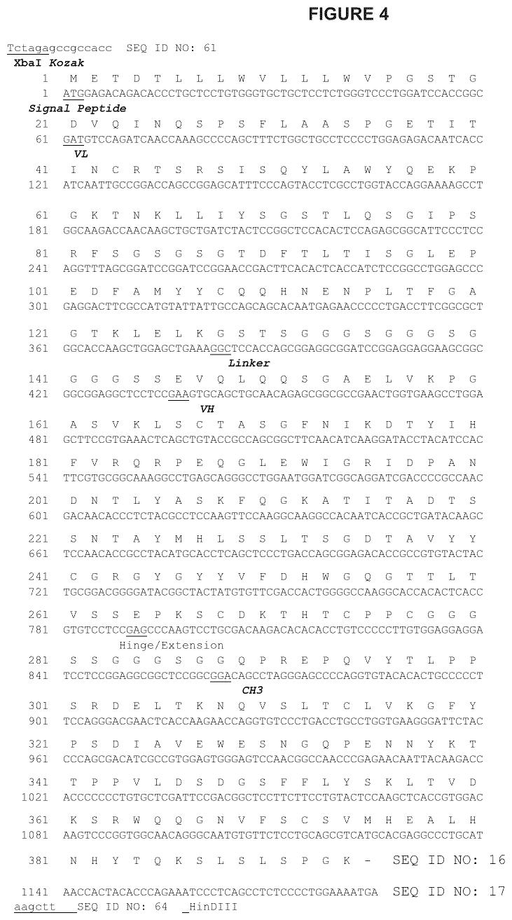

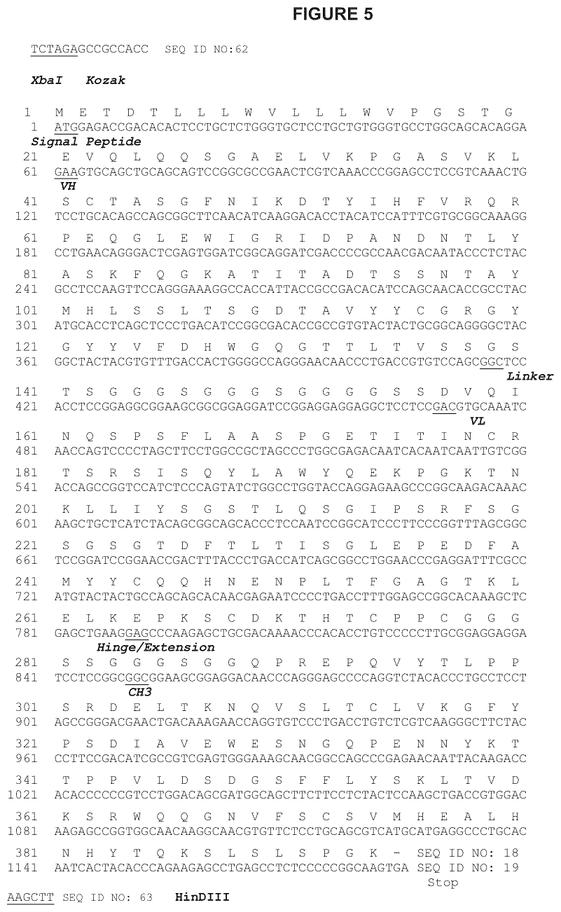

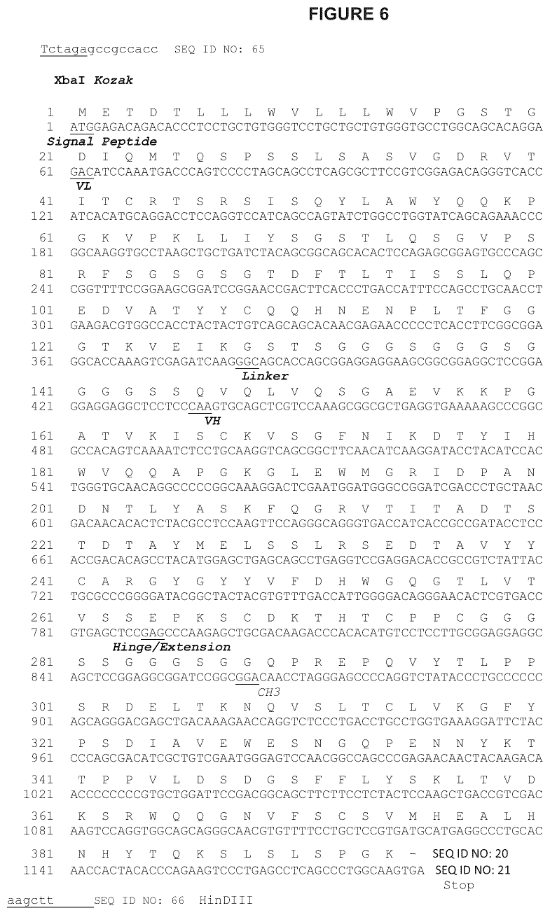

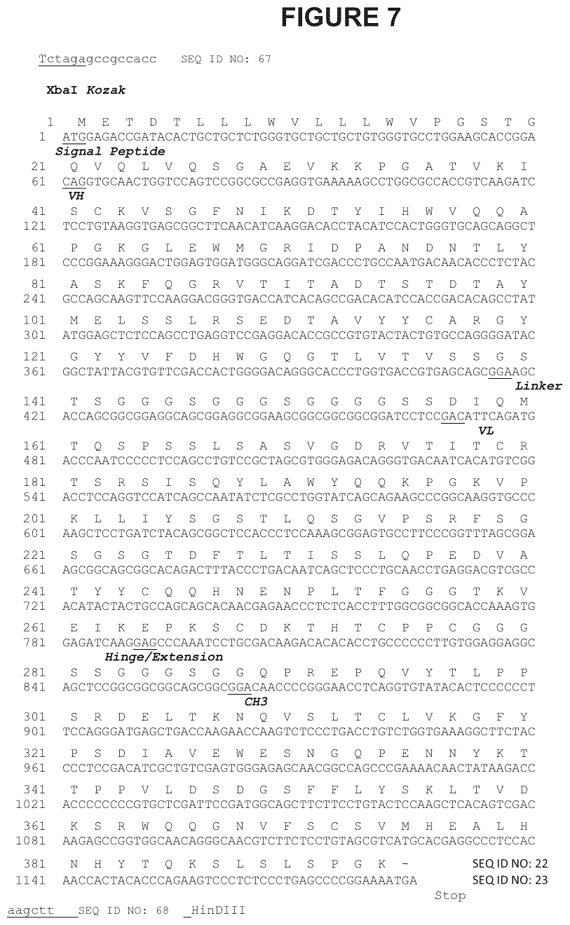

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (for example, 70%, 75%, 80%, 85%, 90%, 95%, 96%, 97%, 98% or 99% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. Some embodiments provided herein provide polypeptides or polynucleotides that are substantially identical to the polypeptides or polynucleotides, respectively, exemplified herein (e.g., the variable regions exemplified in any one FIGS. 2A, 2B, or 4-11, 12C-12I; the CDRs exemplified in any one of FIGS. 2A, 2B, or 12C to 12I; the FRs exemplified in any one of FIGS. 2A, 2B, or 12C-12I; and the nucleic acid sequences exemplified in any one of FIGS. 12A-12I or 4-11). Optionally, the identity exists over a region that is at least about 15, 25 or 50 nucleotides in length, or more preferably over a region that is 100 to 500 or 1000 or more nucleotides in length, or over the full length of the reference sequence. With respect to amino acid sequences, identity or substantial identity can exist over a region that is at least 5, 10, 15 or 20 amino acids in length, optionally at least about 25, 30, 35, 40, 50, 75 or 100 amino acids in length, optionally at least about 150, 200 or 250 amino acids in length, or over the full length of the reference sequence. With respect to shorter amino acid sequences, e.g., amino acid sequences of 20 or fewer amino acids, in some embodiments, substantial identity exists when one or two amino acid residues are conservatively substituted, according to the conservative substitutions defined herein.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2: 482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11. an expectation (E) or 10, M=5, N=−4 and a comparison of both strands. For amino acid sequences. the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, in some embodiments, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The terms “subject,” “patient,” and “individual” interchangeably refer to an entity that is being examined and/or treated. This can include, for example, a mammal, for example, a human or a non-human primate mammal. The mammal can also be a laboratory mammal, e.g., mouse, rat, rabbit, hamster. In some embodiments, the mammal can be an agricultural mammal (e.g., equine, ovine, bovine, porcine, camelid) or domestic mammal (e.g., canine, feline).

The term “therapeutically acceptable amount” or “therapeutically effective dose” interchangeably refer to an amount sufficient to effect the desired result. In some embodiments, a therapeutically acceptable amount does not induce or cause undesirable side effects. A therapeutically acceptable amount can be determined by first administering a low dose, and then incrementally increasing that dose until the desired effect is achieved.

The term “co-administer” refers to the administration of two active agents in the blood of an individual or in a sample to be tested. Active agents that are co-administered can be concurrently or sequentially delivered.

### Antigen Binding Constructs (Including Antibodies and Binding Fragments)

Antigen binding constructs that bind to the target are described herein. An antigen binding construct is a molecule that includes one or more portions of an immunoglobulin or immunoglobulin-related molecule that specifically binds to, or is immunologically reactive with the target molecule.

In some embodiments, the antigen binding constructs allow for the detection of human CD8 which is a specific biomarker found on the surface of a subset of T-cells for diagnostic imaging of the immune system. Imaging of CD8 allows for the in vivo detection of T-cell localization. Changes in T-cell localization can reflect the progression of an immune response and can occur over time as a result various therapeutic treatments or even disease states.

In some embodiments, this is useful for imaging T-cell localization for immunotherapy. Adoptive immunotherapy is a form of therapy where a patient's own T-cells are manipulated in vitro and re-introduced into the patient. For this form of treatment. imaging of T-cells is useful for determining the status of the treatment.

In addition, CD8 plays a role in activating downstream signaling pathways that are important for the activation of cytolytic T cells that function to clear viral pathogens and provide immunity to tumors. CD8 positive T cells can recognize short peptides presented within the MHCI protein of antigen presenting cells. In some embodiments, engineered fragments directed to CD8 can potentiate signaling through the T cell receptor and enhance the ability of a subject to clear viral pathogens and respond to tumor antigens. Thus, in some embodiments, the antigen binding constructs provided herein can be agonists and can activate the CD8 target. In some embodiments, an agonist scFv, minibody, cys-diabody, and/or antibody is provided. In some embodiments, the agonist antigen binding construct includes one or more of the CDRs, heavy chain variable regions, or light chain variable regions provided herein. In some embodiments, the agonist can activate downstream signaling pathways through CD8 for the activation of cytolytic T cells that function to clear viral pathogens and provide immunity to tumors.

In some situations, using full-length antibodies for imaging is not optimal since they typically require imaging times to be scheduled more than 1 week after administration due to the long serum half-lives of full-length antibodies.

Another target-based approach for imaging subtypes of immune cells involves small molecules. For example, one approach for diagnostic imaging of the endogenous immune system has involved the use of small molecule tracers which detect changes in the cell's metabolic pathway such as ¹⁸F-fluoroacetate ([¹⁸F] FAC). Since such tracers detect changes in the metabolic pathway, they target cell populations with elevated metabolic activities which primarily include activated T-cells. The limitation of this approach is that it will only detect the activated subset of T-cells, whereas imaging with anti-CD8 antibody fragments will detect the entire population of CD8 expressing T-cells as the target is expressed on both activated and resting CD8 cells.

The variable regions of the OKT8 antibody were reformatted by protein engineering into various alternative antigen binding constructs. The minibody format is a homodimer with each monomer having a single-chain variable fragment (scFv) linked to the human IgG1 C_{H}3 domain (see FIGS. 1A and 1B). In some embodiments, the scFv is composed of the variable heavy (V_{H}) and light (V_{L}) domains and is connected by an 18 amino acid GlySer-rich linker. In some embodiments, the scFv is tethered to the human IgG1 C_{H}3 domain by the human IgG1 upper and core hinge regions (15 residues) followed by a 10 amino acid GlySer linker. The minibody (V_{H}-V_{L}-C_{H}3) exists as a stable dimer due to the association between the C_{H}3 domains as well as the formation of disulfide bonds within the hinge regions. To allow for secretion of the minibody, a signal sequence is fused at the N-terminus of the variable heavy domain. In some embodiments, the GlySer residues allow for flexibility. In some embodiments, glutamine and/or lysine residues can be added to enhance solubility.

Two variants of the chimeric OKT8 minibody were engineered that differed in the orientation of the variable regions (V_{H} to V_{L} and V_{L} to V_{H}). Every antibody V domain contains two cysteines that form intra-disulfide bonds. The murine OKT8 V_{H} has an extra cysteine in framework 3 (FR3) which may interfere with the expression of the protein as it may lead to aggregation and consequently retention in the endoplasmic reticulum (ER). The chimeric minibodies were made with a serine replacing the extra cysteine in the framework (C84S of the murine V_{H}). In some embodiments, any of the embodiments provided herein can be adjusted to include the C84S adjustment. Tables 0.1, 0.2, and 0.3 provide a summary of come embodiments of the arrangements of various antigen binding constructs provided herein.

TABLE 0.1
Minibodies

| 1 | 2 | 3 | 4 | 5 | 6 |
|---|---|---|---|---|---|
| Name | Leader | Region 1 | Linker | Region 2 | Remainder |
| Chimeric | Leader | murine V_{L} | 18 aa linker | Murine V_{H} | IgG1 |
| IAb_Mb1_CD8 | SEQ ID | SEQ ID | SEQ ID | SEQ ID | hinge/linker- |
| SEQ ID | NO: 34 | NO: 40 | NO: 36 | NO: 44 | C_{H}3 domain |
| NO: | | | | | SEQ ID |
| | | | | | NO: 38 |
| Chimeric | Leader | murine V_{H} | 18 aa linker | Murine V_{L} | IgG1 |
| IAb_Mb2_CD8 | SEQ ID | SEQ ID | SEQ ID | SEQ ID | hinge/linker- |
| | NO: 34 | NO: 44 | NO: 36 | NO: 40 | C_{H}3 domain |
| | | | | | SEQ ID |
| | | | | | NO: 38 |
| Humanized | Leader | hu V_{L} | 18 aa linker | huV_{H} (2^{nd}) | IgG1 |
| IAb_Mb1_CD8 | SEQ ID | SEQ ID | SEQ ID | SEQ ID | hinge/linker- |
| | NO: 34 | NO: 9 | NO: 36 | NO: 6 | C_{H}3 domain |
| | | | | | SEQ ID |
| | | | | | NO: 38 |
| Humanized | Leader | Hu V_{H} (2^{nd}) | 18 aa linker | huV_{L} | IgG1 |
| IAb_Mb2_CD8 | SEQ ID | SEQ ID | SEQ ID | SEQ ID | hinge/linker- |
| | NO: 34 | NO: 6 | NO: | NO: 9 | C_{H}3 domain |
| | | | SEQ ID | | SEQ ID |
| | | | NO: 36 | | NO: 38 |

TABLE 0.2
Affinity Matured Minibodies

| 1 | 2 | 3 | 4 | 5 | 6 |
|---|---|---|---|---|---|
| Name | Leader | Region 1 | Linker | Region 2 | Remainder |
| IAb_Mb1a_CD8 | Leader | huV_{L} | 18aa Linker | huV_{H} | IgG1 |
| | SEQ ID | SEQ ID | SEQ ID | (version a) | hinge/linker- |
| | NO: 34 | NO: 42 | NO: 36 | SEQ ID | C^{H}3 domain |
| | | | | NO: 46 | SEQ ID |
| | | | | | NO: 38 |
| IAb_Mb2a_CD8 | Leader | huV_{H} | 18aa Linker | huV_{L} | IgG1 |
| | SEQ ID | (version a) | SEQ ID | SEQ ID | hinge/linker- |
| | NO: 34 | SEQ ID | NO: 36 | NO: 42 | C^{H}3 domain |
| | | NO: 46 | | | SEQ ID |
| | | | | | NO: 38 |
| IAb_Mb1b_CD8 | Leader | huV_{L} | 18aa Linker | huV_{H} | IgG1 |
| | SEQ ID | SEQ ID | SEQ ID | (version b) | binge/linker- |
| | NO: 34 | NO: 42 | NO: 36 | SEQ ID | C^{H}3 domain |
| | | | | NO: 48 | SEQ ID |
| | | | | | NO: 38 |
| IAb_Mb2b_CD8 | Leader | huV_{H} | 18aa Linker | huV_{L} | IgG1 |
| | SEQ ID | (version b) | SEQ ID | SEQ ID | hinge/linker- |
| | NO: 34 | SEQ ID | NO: 36 | NO: 42 | C^{H}3 domain |
| | | NO: 48 | | | SEQ ID |
| | | | | | NO: 38 |
| IAb_Mb1c_CD8 | Leader | huV_{L} | 18aa Linker | huV_{H} | IgG1 |
| | SEQ ID | SEQ ID | SEQ ID | (version c) | hinge/linker- |
| | NO: 34 | NO: 42 | NO: 36 | SEQ ID | C^{H}3 domain |
| | | | | NO: 50 | SEQ ID |
| | | | | | NO: 38 |
| IAb_Mb2c_CD8 | Leader | huV_{H} | 18aa Linker | huV_{L} | IgG1 |
| | SEQ ID | (version c) | SEQ ID | SEQ ID | hinge/linker- |
| | NO: 34 | SEQ ID | NO: 36 | NO: 42 | C^{H}3 domain |
| | | NO: 50 | | | SEQ ID |
| | | | | | NO: 38 |
| IAb_Mb1d_CD8 | Leader | huV_{L} | 18aa Linker | huV_{H} | IgG1 |
| | SEQ ID | SEQ ID | SEQ ID | (version d) | hinge/linker- |
| | NO: 34 | NO: 42 | NO: 36 | SEQ ID | C_{H}3 domain |
| | | | | NO: 52 | SEQ ID |
| | | | | | NO: 38 |
| IAb_Mb2d_CD8 | Leader | huV_{H} | 18aa Linker | huV_{L} | IgG1 |
| | SEQ ID | (version d) | SEQ ID | SEQ ID | binge/linker- |
| | NO: 34 | SEQ ID | NO: 36 | NO: 42 | C_{H}3 domain |
| | | NO: 52 | | | SEQ ID |
| | | | | | NO: 38 |

TABLE 0.3
Cys-Diabodies

| 1 | 2 | 3 | 4 | 5 | 6 |
|---|---|---|---|---|---|
| Name | Leader | Region 1 | Linker | Region 2 | Remainder |
| IAb_Cys- | Leader | huV_{L} | 5aa Linker | huV_{H} | Cys Tail |
| Db1b_CD8 | SEQ ID | SEQ ID | SEQ ID | (version b) | SEQ ID |
| | NO: 26 | NO: 42 | NO: 28 | SEQ ID | NO: 32 |
| | | | | NO: 48 | |
| IAb_Cys- | Leader | huV_{H} | 5aa Linker | huV_{L} | Cys Tail |
| Db2b_CD8 | SEQ ID | (version b) | SEQ ID | SEQ ID | SEQ ID |
| | NO: 26 | SEQ ID | NO: 28 | NO: 42 | NO: 32 |
| | | NO: 48 | | | |
| IAb_Cys- | Leader | huV_{L} | 8aa Linker | huV_{H} | Cys Tail |
| Db3b_CD8 | SEQ ID | SEQ ID | SEQ ID | (version a) | SEQ ID |
| | NO: 26 | NO: 42 | NO: 30 | SEQ ID | NO: 32 |
| | | | | NO: 48 | |
| IAb_Cys- | Leader | huV_{H} | 8aa Linker | huV_{L} | Cys Tail |
| Db4b_CD8 | SEQ ID | (version a) | SEQ ID | SEQ ID | SEQ ID |
| | NO: 26 | SEQ ID | NO: 30 | NO: 42 | NO: 32 |
| | | NO: 48 | | | |

Depicted in Tables 0.1, 0.2, and 03 are arrangements of sequences for monomers that can be used in minibodies (Table 0.1 and 0.2) and cys-diabodies (Table 0.3). Each row of the table represents the sequence of a monomer construct, with left-to-right representing N-terminus to C-terminus. In some embodiments, the shown sequences of each monomer construct are directly linked to each other. Thus, in some embodiments, the construct can include any of the constructs on a single row in Table 0.1, Table 0.2, or Table 0.3. In some embodiments, the constructs can include any combination in Table 0.1, Table 0.2, or Table 0.3. In some embodiments, for example, the first item in the first row, column 2 can be combined with the first row, column 3 to the first row column 4, to the first row column 5, to the first row, column 6. In some embodiments, column 3 and column 6 can be swapped with one another. In some embodiments, the first item in the first row, column 2 can be combined with the first row, column 3 to the second row column 4, to the second row column 5, to the second row, column 6. Thus, the tables represent all possible combinations, both within a single row and across various rows (and with columns swapped).

In some embodiments, an antigen binding construct includes a heavy chain CDR1 (HCDR1) of the HCDR1 in SEQ ID NOs:3, 6, 44, 46, 48, 50, or 52; a heavy chain CDR2 (HCDR2) of the HCDR2 in SEQ ID NOs:3, 6, 44, 46, 48, 50, or 52; a heavy chain CDR3 (HCDR3) of the HCDR3 in SEQ ID NOs:3, 6, 44, 46, 48, 50, or 52; a light chain CDR1 (LCDR1) of the LCDR1 in SEQ ID NOs:9 or 42; a light chain CDR2 (LCDR2) of the LCDR2 in SEQ ID NOs:9 or 42; and/or a light chain CDR3 (LCDR3) of the LCDR3 in SEQ ID NOs:9 or 42. In some embodiments, the antigen binding construct includes 6, 5, 4, 3, 2, or 1, the above CDRs (some embodiments of the CDRs are indicated in FIGS. 2A, 2B, 12C-121). In some embodiments, the antigen binding construct includes HCDR3. In some embodiments, the antigen binding construct binds specifically to the target molecule. In some embodiments, the antigen binding construct competes for binding with one or more of the antibodies having the herein provided CDRs. In some embodiments, the antigen binding construct includes at least the 3 heavy chain CDRs noted herein. In some embodiments, the antigen binding construct includes heavy chain CDR3. In some embodiments, the antigen binding construct further includes any one of the heavy chain CDR2 sequences provided herein.

In some embodiments, the antigen binding construct is human or humanized. In some embodiments, the antigen binding construct includes at least one human framework region, or a framework region with at least about 80% sequence identity, for example at least about 80%, 85%, 90%, 93%, 95%, 97%, or 99% identity to a human framework region. In some embodiments the antigen binding construct includes a heavy chain FR1 (HFR1) of the HFR1 in SEQ ID NO:3, 6, 44, 46, 48, 50, or 52; a heavy chain FR2 (HFR2) of the HFR2 in SEQ ID NO:3, 6, 44, 46, 48, 50, or 52; a heavy chain FR3 (HFR3) of the HFR3 in SEQ ID NO:3, 6, 44, 46, 48, 50, or 52; a heavy chain FR4 (HFR4) of the HFR4 in SEQ ID NO:3, 6, 44, 46, 48, 50, or 52; a light chain FR1 (LFR1) of the LFR 1 in SEQ ID NO:9 or 42; a light chain FR2 (LFR2) of the LFR2 in SEQ ID NO:9 or 42; a light chain FR3 (LFR3) of the LFR3 in SEQ ID NO:9 or 42; and a light chain FR4 (LFR4) of the LFR4 in SEQ ID NO:9 or 42. In some embodiments, the antigen binding construct includes 8, 7, 6, 5, 4, 3, 2, or 1 of the listed FRs.

In some embodiments, the antigen binding construct includes a detectable marker. In some embodiments, the antigen binding construct includes a therapeutic agent.

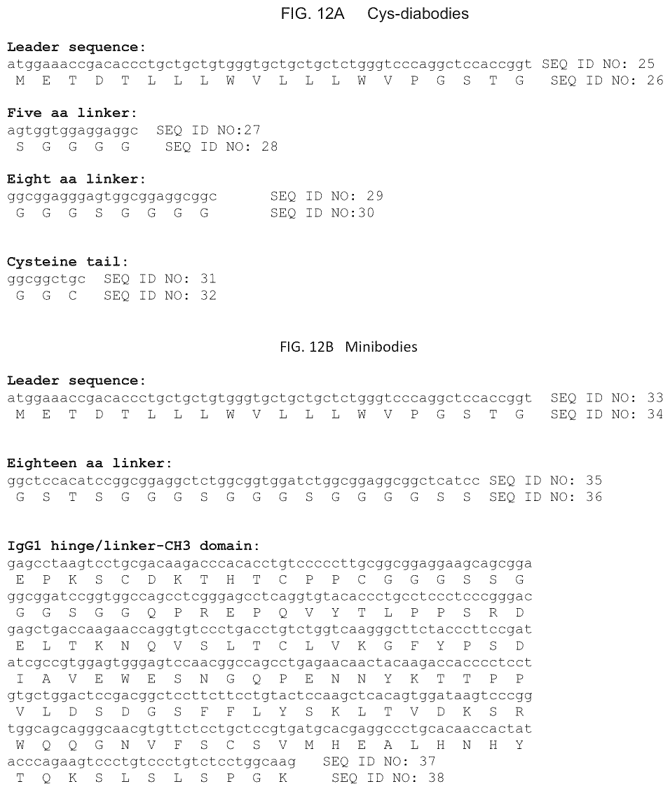

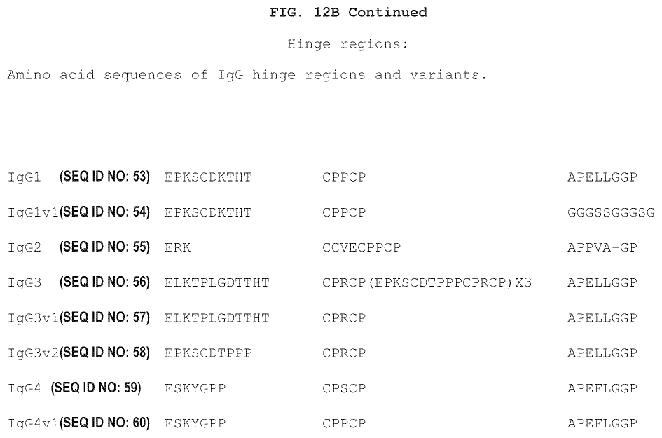

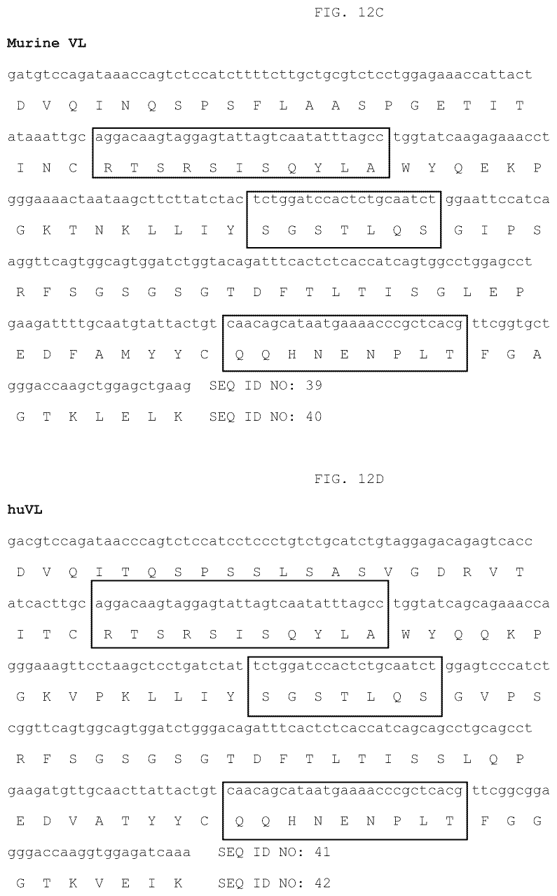

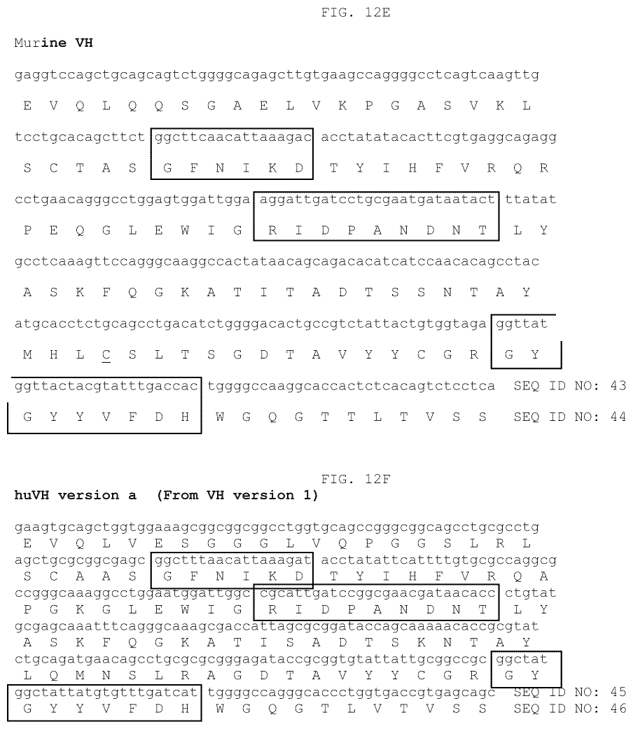

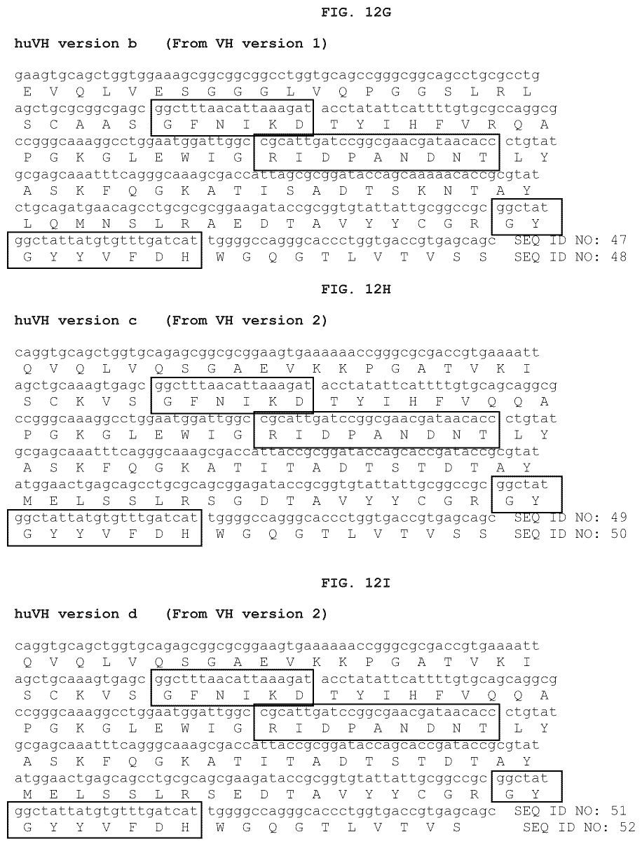

In some embodiments, the antigen binding construct is bivalent. Bivalent antigen binding construct can include at least a first antigen binding domain, for example a first scFv, and at least a second antigen binding domain, for example a second scFv. In some embodiments, a bivalent antigen binding construct is a multimer that includes at least two monomers, for example at least 2, 3, 4, 5, 6, 7, or 8 monomers, each of which has an antigen binding domain. In some embodiments, the antigen binding construct is a minibody. In some embodiments, the antigen binding construct is a diabody, including, for example, a cys-diabody. The scFv, and/or minibody and/or the cys-diabody can include any of the CDR and heavy chain variable region and/or light chain variable region embodiments provided herein (for example, the CDR sequences provided in FIGS. 2A, 2B, and 12C-12I). In some embodiments, the antigen binding construct is a monovalent scFv. In some embodiments, a monovalent scFv is provided that includes the HCDR1 in the HCDR1 of FIG. 2A, FIG. 12E-12I, or SEQ ID NO:3, 6, 44, 46, 48, 50, or 52; the HCDR2 in the HCDR2 of FIG. 2A, FIG. 12E-12I, or SEQ ID NO:3, 6, 44, 46, 48, 50, or 52; the HCDR3 in the HCDR3 of FIG. 2A, FIG. 12E-12I, or SEQ ID NO:3, 6, 44, 46, 48, 50, or 52; the LCDR1 in the LCDR1 of FIG. 2B, FIG. 12C, FIG. 12D, SEQ ID NO:9 or 42; the LCDR2 in the LCDR2 of FIG. 2B, FIG. 12C, FIG. 12D, SEQ ID NO:9 or 42, and the LCDR3 in the LCDR3 of FIG. 2B, FIG. 12C, FIG. 12D, SEQ ID NO:9 or 42. In some embodiments, the monovalent scFv includes the heavy chain variable region of the heavy chain variable region in FIG. 2A, FIGS. 4-11, FIGS. 12C-121, or SEQ ID NO:3, 6, 44, 46. 48, 50, or 52. In some embodiments, the monovalent scFv includes the light chain variable region of the light chain variable region in FIG. 2B, 4-11, 12C, 12D, SEQ ID NO:9, 42, or 40. In some embodiments, the monovalent scFv includes the heavy chain variable region of the heavy chain variable region in FIG. 2A, FIGS. 4-11, FIGS. 12C-12I, or SEQ ID NO:3, 6, 44, 46, 48, 50, or 52 and the light chain variable region of the light chain variable region in in FIG. 2B, 4-11, 12C, 12D, SEQ ID NO:9, 42, or 40.

In some embodiments, the antigen binding construct is bispecific. Bispecific antibodies can include at least a first binding domain, for example an scFv that binds specifically to a first epitope, and at least a second binding domain, for example an scFv that binds specifically to a second epitope. Thus, bispecific antigen binding constructs can bind to two or more epitopes. In some embodiments, the first epitope and the second epitope are part of the same antigen, and the bispecific antigen binding construct can thus bind to two epitopes of the same antigen. In some embodiments, the first epitope is part of a first antigen, and the second epitope is part of a second antigen, and the bispecific antigen binding construct can thus bind to two different antigens. In some embodiments, the antigen binding construct binds to two epitopes simultaneously.

In some embodiments, the antigen binding construct has a heavy chain variable region of the heavy chain variable region in SEQ ID NO:3, 6, 16, 18, 20, 22, 44, 46, 48, 50, or 52. In some embodiments, the antigen binding construct has a heavy chain variable region that includes a sequence with at least about 80% identity, for example at least about 80%, 85%, 86%, 87%, 88%, 89%, 90%, 91%, 92%, 93%, 94%, 95%, 96%, 97%, 98%, or 99% identity to SEQ ID NO:3. In some embodiments, the antigen binding construct has a heavy chain variable region that includes a sequence with at least about 80% identity, for example at least about 80%, 85%, 86%. 87%, 88%, 89%, 90%, 91%, 92%, 93%, 94%, 95%, 96%. 97%, 98%, or 99% identity to SEQ ID NO:6. In some embodiments, the antigen binding construct has a heavy chain variable region that includes a sequence with at least about 80% identity, for example at least about 80%, 85%, 86%, 87%, 88%, 89%, 90%, 91%, 92%, 93%, 94%, 95%, 96%, 97%, 98%, or 99% identity to SEQ ID NO:44. In some embodiments, the antigen binding construct has a heavy chain variable region that includes a sequence with at least about 80% identity, for example at least about 80%, 85%, 86%, 87%, 88%, 89%, 90%, 91%, 92%, 93%, 94%, 95%, 96%, 97%, 98%, or 99% identity to SEQ ID NO:46. In some embodiments, the antigen binding construct has a heavy chain variable region that includes a sequence with at least about 80% identity, for example at least about 80%, 85%, 86%, 87%, 88%, 89%, 90%, 91%, 92%, 93%, 94%, 95%, 96%, 97%, 98%, or 99% identity to SEQ ID NO:48. In some embodiments, the antigen binding construct has a heavy chain variable region that includes a sequence with at least about 80% identity, for example at least about 80%, 85%, 86%, 87%, 88%, 89%, 90%, 91%, 92%, 93%, 94%, 95%, 96%, 97%, 98%, or 99% identity to SEQ ID NO:50. In some embodiments, the antigen binding construct has a heavy chain variable region that includes a sequence with at least about 80% identity, for example at least about 80%, 85%, 86%, 87%, 88%, 89%, 90%, 91%, 92%, 93%, 94%, 95%, 96%, 97%, 98%, or 99% identity to SEQ ID NO:52.

In some embodiments, the antigen binding construct has a light chain variable region that includes SEQ ID NO:9, 16, 18, 20, 22, 40, or 42. In some embodiments, the antigen binding construct has a light chain variable region that includes a sequence with least about 80% identity, for example at least about 80%, 85%, 86%, 87%, 88%, 89%, 90%. 91%, 92%, 93%, 94%, 95%. 96%, 97%, 98%, or 99% identity to SEQ ID NO:9. In some embodiments, the antigen binding construct has a light chain variable region that includes a sequence with least about 80% identity, for example at least about 80%, 85%, 86%, 87%, 88%, 89%, 90%, 91%, 92%, 93%, 94%, 95%, 96%, 97%, 98%, or 99% identity to SEQ ID NO:40. In some embodiments, the antigen binding construct bas a light chain variable region that includes a sequence with least about 80% identity, for example at least about 80%, 85%, 86%, 87%, 88%, 89%, 90%, 91%, 92%, 93%, 94%, 95%, 96%, 97%, 98%, or 99% identity to SEQ ID NO:42. In some embodiments, the antigen binding construct is a human antigen binding construct and has a heavy chain variable region, a light chain variable region, or a heavy and light chain that is at least as identical as at least the heavy and/or light chain variable sequences noted above.

Some embodiments provided herein include an antigen binding construct that competes for binding to the target molecule with one or more antigen binding constructs provided herein. In some embodiments, the competing antigen binding construct binds to the same epitope on the target molecule as the reference antigen binding construct. In some embodiments, the reference antigen binding construct binds to a first epitope of the target molecule, and the competing antigen binding construct binds to a second epitope of the target molecule, but interferes with binding of the reference antigen binding construct to the target molecule, for example by sterically blocking binding of the reference antigen binding construct, or by inducing a conformational change in the target molecule. In some embodiments, the first epitope overlaps with the second epitope. In some embodiments. columns 3 and 5 of Tables 0.1 and/or 0.2 can be swapped. In some embodiments, any of the heavy chains variable regions provided herein can be combined with any of the light chain variable regions herein for a scFv, minibody, and/or diabody. In some embodiments, any of the heavy and/or light chain variable regions (columns 3 and 5) in tables 0.1, 0.2, and 0.3 can be exchanged with one another or another light or heavy chain variable region, to produce an antigen binding construct (such as a scFv, a cys-diabody, a minibody, or an antibody).

In some embodiments, the minibody and cys-diabody formats have advantageous pharmacokinetic characteristics for diagnostic imaging and certain therapeutic applications while maintaining the high binding affinity and specificity of a parental antibody. Compared to imaging with the full-length parental antibody, the pharmacokinetics are more desirable for these fragments in that they are able to target the antigen and then rapidly clear the system for rapid high-contrast imaging. In some embodiments, the shorter serum half lives for the minibody and the cys-diabody allow for imaging to occur over a range of times, approximately 8-48 hours post injection for the minibody and 2-24 hours post-injection for the cys-diabody. The rapid serum clearance together with better tissue penetration can allow for same day imaging, providing a significant advantage in the clinic with respect to patient care management.

In addition, the cys-diabody antibody format features the C-terminus cysteine tail. These two sulfhydryl groups (following mild reduction) provide a strategy for site-specific conjugation of functional moieties such as radiolabels that need not interfere with the cys-diabody's binding activity.

### Diabodies that Bind to the Target Molecule

In some embodiments, the antigen binding construct can be a diabody. The diabody can include a first polypeptide chain which includes a heavy (V_{H}) chain variable domain connected to a light chain variable domain (V_{L}) on the first polypeptide chain. In some embodiments, the light and heavy variable chain domains can be connected by a linker. The linker can be of the appropriate length to reduce the likelihood of pairing between the two domains on the first polypeptide chain and a second polypeptide chain comprising a light chain variable domain (V_{L}) linked to a heavy chain variable domain V_{H} on the second polypeptide chain connected by a linker that is too short to allow significant pairing between the two domains on the second polypeptide chain.

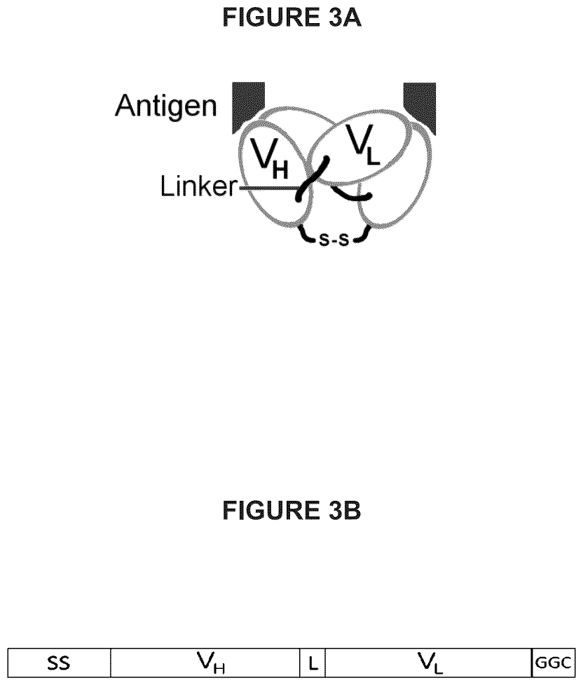

In some embodiments, the appropriate length of the linker encourages chain pairing between the complementary domains of the first and the second polypeptide chains and can promote the assembly of a dimeric molecule with two functional antigen binding sites. Thus, in some embodiments, the diabody is bivalent. In some embodiments. the diabody can be a cysteine linked diabody (a Cys-Db). A schematic of a Cys-Db binding to two antigen sites is illustrated in FIG. 3A and 3B.

In some embodiments, the linker can be a peptide. In some embodiments, the linker can be any suitable length that promotes such assembly, for example, between 1 and 20 amino acids, such as 5 and 10 amino acids in length. As described further herein, some cys-diabodies can include a peptide linker that is 5 to 8 amino acids in length. In some embodiments, the linker need not be made from, or exclusively from amino acids, and can include, for example, modified amino acids (see, for example, Increased Resistance of Peptides to Serum Proteases by Modification of their Amino Groups, Rossella Galati, Alessandra Verdina, Giuliana Falasca, and Alberto Chersi, (2003) Z. Naturforsch, 58c, 558-561). In some embodiments, the linker can be 1, 2, 3, 4, 5, 6, 7, 8, 9, 10, 11, 12, 13, 14, 15, 16, 17, 18, 19, or 20 amino acids in length. In some embodiments, the linker can be from 2 to 30 angstroms in length, for example 2.5 to 27 angstroms.

In some embodiments, the antigen binding construct includes a humanized cys-diabody. The humanized cys-diabody can include a single-chain variable fragment (scFv) that includes a variable heavy (V_{H}) domain linked to a variable light (V_{L}) domain, and a C-terminal cysteine. In some embodiments, the humanized cys-diabody is a homodimer. In some embodiments, the humanized diabody is a heterodimer. In some embodiments, individual monomers are provided that each have a cysteine terminal residue.

In some embodiments, the scFv of the humanized cys-diabody has a V_{H}-V_{L} orientation or a V_{L}-V_{H} orientation. As used herein, a V_{H}-V_{L} (which may also be referred to herein as “V_{H}V_{L}”) orientation means that the variable heavy domain (V_{H}) of the scFv is upstream from the variable light domain (V_{L}) and a V_{L}V_{H} orientation means that the V_{L} domain of the scFv is upstream from the V_{H} domain. As used herein, “upstream” means toward the N-terminus of an amino acid or toward the 5′ end of a nucleotide sequence.

The antibody variable regions can be linked together by a linker as described herein. In some embodiments, the linker is a GlySer linker as described herein.

In some embodiments, the cys-diabody includes a detectable marker.

In some embodiments, the cys-diabody includes a pair of monomers. Each monomer can include a polypeptide. In some embodiments, the polypeptides of the monomers are identical (for example, cys-diabody can be a homodimer). In some embodiments, the polypeptides of the monomers are different (for example, the cys-diabody can be a heterodimer).

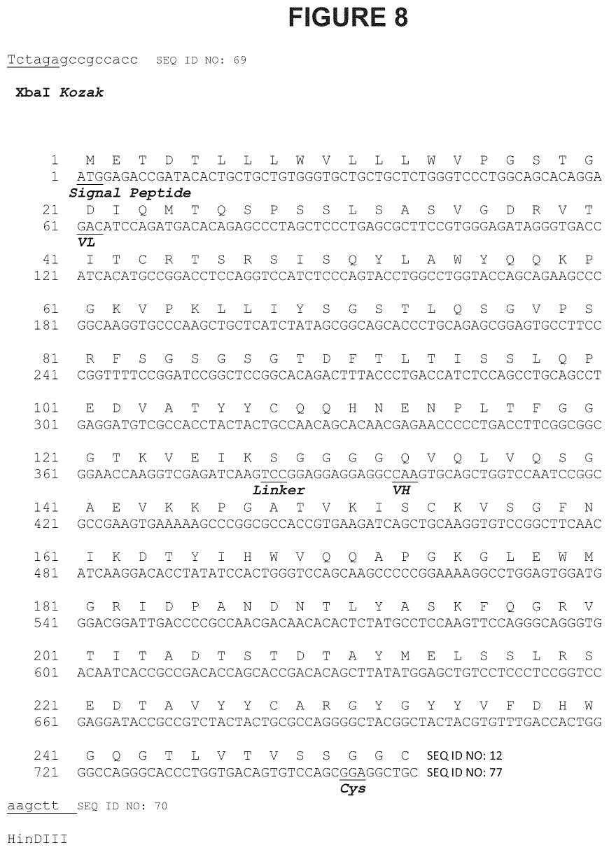

In some embodiments, the polypeptide of the monomer includes SEQ ID NO:12 (See FIG. 8). In some embodiments, the polypeptide of the monomer includes a sequence with least about 80% identity, for example at least about 80%, 85%, 86%, 87%, 88%, 89%, 90%, 91%, 92%, 93%, 94%, 95%, 96%, 97%, 98%, or 99% identity to SEQ ID NO:12 (cys-diabody (V_{L}-5-V_{H})).

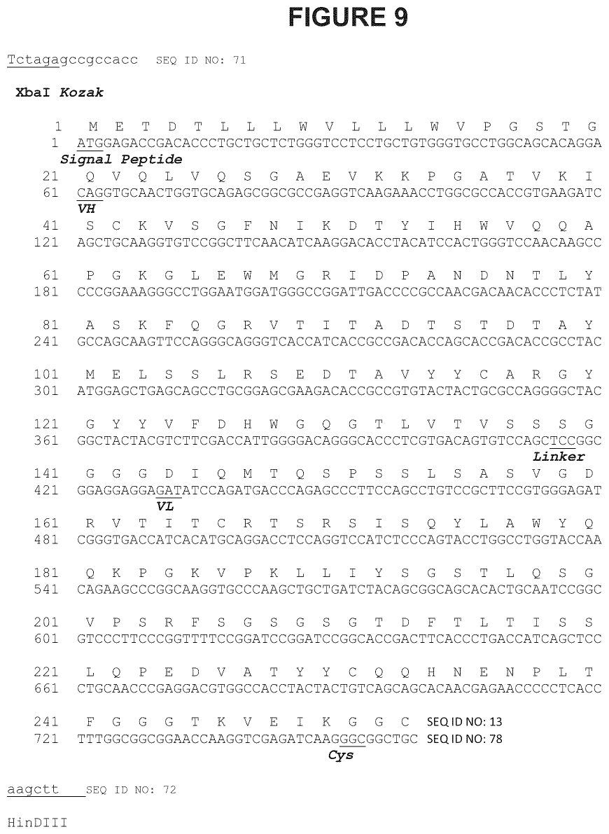

In some embodiments, the polypeptide of the monomer includes SEQ ID NO:13 (See FIG. 9). In some embodiments, the polypeptide of the monomer includes a sequence with least about 80% identity, for example at least about 80%, 85%, 86%, 87%, 88%, 89%, 90%, 91%, 92%, 93, 94, 95%, 96%, 97%, 98%, or 99% identity to SEQ ID NO:13 (cys-diabody (V_{H}-5-V_{L})).

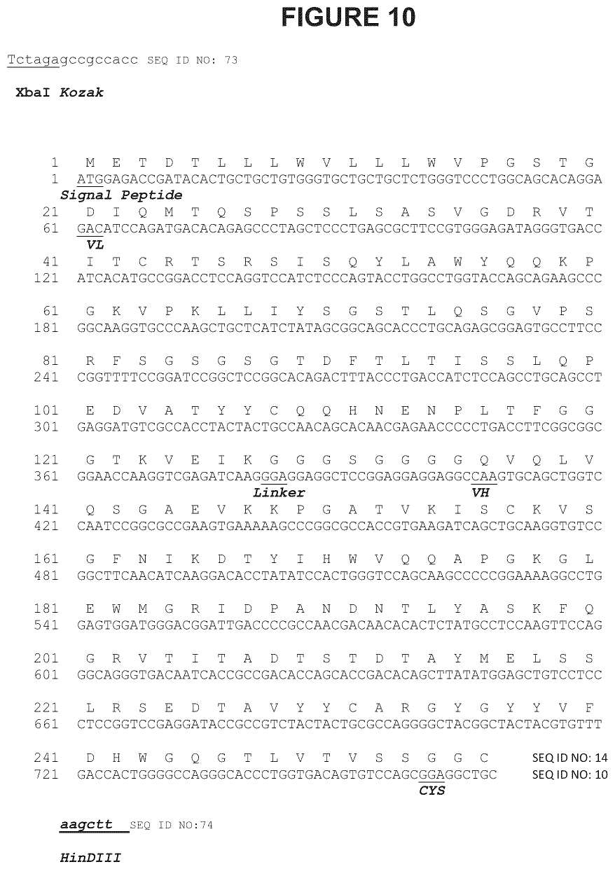

In some embodiments, the polypeptide of the monomer includes SEQ ID NO:14 (V_{L}-8-V_{H}) (See FIG. 10). In some embodiments, the polypeptide of the monomer includes a sequence with least about 80% identity, for example at least about 80%, 85%, 86%, 87%, 88%, 89%, 90%, 91%, 92%, 93, 94, 95%, 96%, 97%, 98%, 99%, or 100% identity to SEQ ID NO:14.

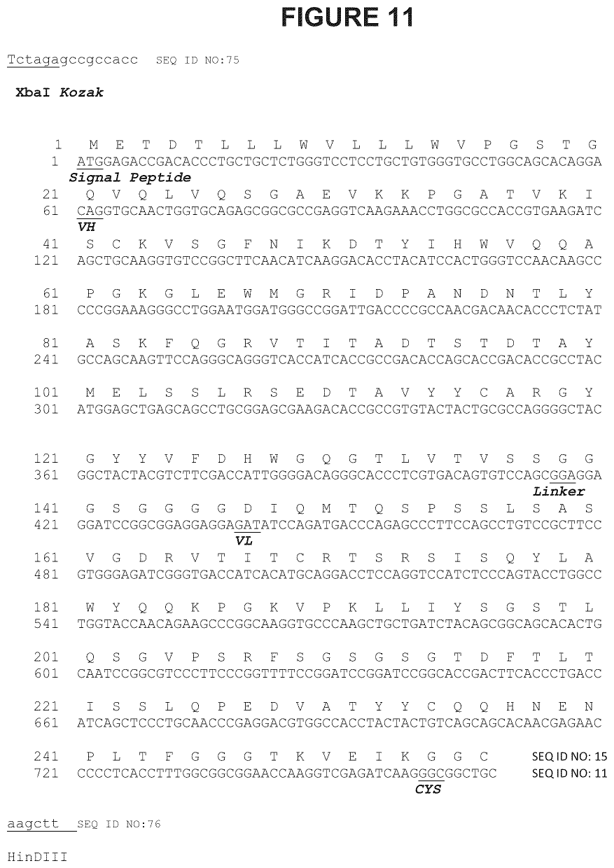

In some embodiments, the polypeptide of the monomer includes SEQ ID NO:15 (humanized OKT8 cys-diabody (V_{H}-8-V_{L})) (See FIG. 11). In some embodiments, the polypeptide of the monomer includes a sequence with least about 80% identity, for example at least about 80%, 85%, 86%, 87%, 88%, 89%, 90%, 91%, 92%, 93, 94, 95%, 96%, 97%, 98%, 99%, or 100% identity to SEQ ID NO:15.

In some embodiments, the polypeptide of the monomer includes any of the combined sections as indicated in Table 0.3, including polypeptides of the monomer with a sequences of at least about 80% identity, for example at least about 80%, 85%, 86%, 87%, 88%, 89%, 90%, 91%, 92%, 93, 94, 95%, 96%, 97%, 98%, 99%, or 100% identity to the monomers as set forth in Table 0.3.

In some embodiments, the cysteines are cross-linked with one another. In some embodiments, the cysteines are reduced, and thus, these tail forming cysteines do not form a disulfide bond with one another. In some embodiments, one or more of the “tail forming” cysteines form a covalent bond with one or more detectable marker, such as a fluorescent probe.

As will be appreciated by those of skill in the art, while the present disclosure generally references “cys-diabodies” alternative arrangements can be employed to achieve the same or similar ends. In some embodiments, any covalently modifiable moiety can be employed in place of one or more of the cysteines. For example, this can include a GlySer linker, a GlyLeu linker, and/or an insert cysteine after a short tag. In some embodiments, the connection can be established via a coiled coil or a leucine zipper. In some embodiments, the “tail” itself can include functional groups on its end so that it can selectively bind to a desired residue and/or location at the ends of each of the polypetides, in place of the disulfide bond itself. In some embodiments, rather than the tail providing space between the two polypeptide chains, the covalently modifiable moieties can be attached directly to the end of the heavy or light chain polypeptide, but the two covalently modifiable moieties can be connected by a linker.

In some embodiments, a chimeric cys-diabody that binds to the target molecule is provided. In some embodiments, the chimeric cys-diabody includes a monomer in the V_{L}-V_{H} format, and includes the sequence of SEQ ID NO:12 or 14, or a sequence having at least about 80% identity thereto, for example at least about 80%, 85%, 90%, 95%, 96%. 97%, 98%, or 99% % identity thereto. In some embodiments, the chimeric cys-diabody includes a monomer in the V_{H}-V_{H} format, and includes the sequence of SEQ ID NO:13 or 15, or a sequence having at least about 80% identity thereto, for example at least about 80%, 85%, 90%, 95%, 96%, 97%, 98%, or 99% % identity thereto.

In some embodiments, any of the constructs provided herein (including those arrangements noted as cys-diabody embodiments, can be provided as a scFv embodiment. In such embodiments, the construct can still include the cysteine on the tail, but simply not be cross-linked. In other embodiments, the construct need not have the cysteine in a tail or the tail at all.

### Linker and/or Tail Options

In some embodiments, for individual antibodies, the heavy and light chain variable domains can associate in different ways. For this reason, the use of different linker lengths allows for conformational flexibility and range-of-motion to ensure formation of the disulfide bonds.

In some embodiments, the two linker lengths can be somewhere between (and including) about 1 to 50 amino acids, for example, 2 to 15, 2 to 14, 3 to 13, 4 to 10, or 5 amino acids to 8 amino acids. In some embodiments, each linker within a pair for a diabody can be the same length. In some embodiments, each linker within the pair can be a different length. In some embodiments, any combination of linker length pairs can be used, as long as they allow and/or promote the desired combinations. In some embodiments, a modified amino acid can be used.

FIGS. 8-11 provide four Cys-Db variants, V_{H}-5-V_{L}, V_{H}-8-V_{L}, V_{L}-5-V_{H}, and VL8VH (sec FIGS. 8-11, and Table 0.3). Producing and testing the expression and binding of all four variants allows for identification of a desired format for protein production for each new Cys-Db. Evaluating the set of variants can help to make certain that a high-quality, stable protein is produced where the disulfide bridge is available. Therefore, engineering a Cys-Db can involve using two distinct linker lengths, not one—as in the minibody, as well as both orientations of the variable regions, V_{H}-V_{L} and V_{L}-V_{H}.

In some embodiments, the linker is a GlySer linker. The GlySer linker can be a polypeptide that is rich in Gly and/or Ser residues. In some embodiments, at least about 40% of the amino acid residues of the GlySer linker are Gly, Ser, or a combination of Gly and Ser, for example at least about 40%, 50%, 60%, 70%, 80%, or 90%. In some embodiments, the GlySer linker is at least about 2 amino acids long, for example at least about 3, 4, 5, 6, 7, 8, 9, 10, 11, 12, 13, 14, 15, 16, 17, 18, 19, 20, 25, 30, 35, or 40 amino acids long. In some embodiments, the linker includes at least one of SEQ ID NO:28, 30, and/or 36.

In some embodiments, a cysteine is added at the C-terminus of the diabody. This cysteine can allow the diabody complex to form covalent cysteine bonds and provides the option for available sulfur residues for site-specific conjugation of functional moieties such as radiolabels. In some embodiments, a terminal end of the antibody itself is altered so as to contain a cysteine. In some embodiments, a tail sequence, for example (Gly-Gly-Cys) is added at the C-terminus. In some embodiments, the cysteine tail sequence allows two monomers of a cys-diabody to form disulfide bonds with each other. In some embodiments, the cysteine tail sequence allows a cys-diabody to form disulfide linkages with a detectable moiety such as a detectable marker and/or therapeutic agent. The sulfhydryl groups of the cysteine tail can undergo mild reduction prior to site-specific conjugation of a desired functional moiety, for example a detectable marker and/or therapeutic agent. In some embodiments, the tail is at least about 1 amino acid long, for example at least about 1, 2, 3, 4, 5, 6, 7, 8, 9, 10, 11, 12, 13, 14, 15, 16, 17, 18, 19, 20, 25, 30, 35, or 40 amino acids long. In some embodiments, the tail includes at least one of SEQ ID NO:32. In some embodiments, the tail is 3 to 8 amino acids in length. In some embodiments, the tail can and/or include a coiled coil and/or a leucine zipper. As noted above, in some embodiments, the cysteine is located at the c-terminus; however, this does not require that the cysteine be located as the last c-terminal amino acid. Instead, this denotes that the cysteine can be part of any of the residues that are located in the C-terminus of the protein.

In some embodiments, the linking option between the two C-terminuses can be achieved by a cysteine, for direct and/or indirect, cross-linking.

### Minibodies that Bind to the Target Molecule

A “minibody” as described herein includes a homodimer, wherein each monomer is a single-chain variable fragment (scFv) linked to a human IgG1 C_{H}3 domain by a linker, such as a hinge sequence. In some embodiments, the hinge sequence is a human IgG1 hinge sequence as shown in FIG. 12B, SEQ ID NOs:53-60.

In some embodiments, the hinge sequence is an artificial hinge sequence. In some embodiments, the hinge sequence can be an IgG hinge from any one or more of the four classes. The artificial hinge sequence may include a portion of a human IgG1 hinge and a GlySer linker sequence.

In some embodiments, the artificial hinge sequence includes approximately the first 14 or 15 residues of the human IgGI hinge followed by a linker sequence. In some embodiments, the linker can be any of those provided herein. In some embodiments, the linker can be a GlySer linker sequence that is 6, 7, 8, 9 or 10 amino acids in length. In some embodiments, the artificial hinge sequence includes approximately the first 15 residues of the IgGI hinge followed by a GlySer linker sequence that is about 10 amino acids in length. In some embodiments, association between the C_{H}3 domains causes the minibody to exist as a stable dimer.

In some embodiments, the minibody scFv sequence can include CDR and/or FR, and or variable region sequences that are similar and/or the same to a diabody sequence described herein (for Example, as found in FIGS. 2A, 2B, 4, 5, 6, 7, 8, 9, 10, 11, and 12C-12I and tables 0.1 and 0.2). In some embodiments, the minibody scFv has a sequence (CDR, CDRs, full set of 6 CDRS, heavy chain variable region, light chain variable region, heavy and light chain variable regions, etc) that is at identical to a scFv of a cys-diabody described herein.

In some embodiments, the minibody has a sequence that is at least about 80% identical to a sequence in SEQ ID NO:3, 6, 9, 16, 18, 20, 22, 34, 36, 38, 53-60, 40, 42, 44, 46, 48, 50, and 52, and/or the sequence for the arrangements in Tables 0.1 and/or 0.2, for example at least about 80%, 85%, 86%, 87%, 88%, 89%, 90%, 91%, 92%, 93, 94, 95%, 96%, 97%, 98%, or 99% identity.

In some embodiments, the minibody has a variable chain region that is at least about 80% identical to a sequence in SEQ ID NO:3, 6, 9, 16, 18, 20, 22, 40, 42, 44, 46, 48, 50, and 52, for example at least about 80%, 85%, 86%, 87%, 88%, 89%, 90%, 91%, 92%, 93, 94, 95%, 96%, 97%, 98%, or 99% identity.

The scFv can have a V_{H}-V_{L} or a V_{L}-V_{H} orientation. In some embodiments, the V_{H} and V_{H} are linked to each other by an amino acid linker sequence. The amino acid linker can be a linker as described herein. In some embodiments, the linker is GlySer-rich and approximately 15-20 amino acids in length. In another embodiment, the linker is GlySer rich and is 18 amino acids in length. In some embodiments, the linker length varies between (and including) about 1 to 50 amino acids, for example, 2 to 30, 3 to 20, 4 to 15, or 5 amino acids to 8 amino acids. In some embodiments, the minibody scFv has a sequence that is at least about 80% identical to a scFv of a cys-diabody described herein, for example at least about 80%, 85%, 86%, 87%, 88%, 89%, 90%, 91%, 92%, 93, 94, 95%, 96%, 97%, 98%, or 99% identity. The scFv can have a V_{H}V_{L} or a V_{L}V_{H} orientation.

In some embodiments, each monomer of the minibody includes the following elements, from N-terminus to C-terminus: (a) an scFv sequence that includes a V_{H} domain linked to a V_{L} domain and that binds to the target molecule, (b) a hinge-extension domain comprising a human IgG1 hinge region, and (c) a human IgG C_{H}3 sequence. In some embodiments, each monomer of the minibody includes an IgG2, an IgG3, or an IgG4 C_{H}3. In some embodiments, the minibody is encoded by a nucleic acid can be expressed by a cell, a cell line or other suitable expression system as described herein. Thus, a signal sequence can be fused to the N-terminus of the scFv to enable secretion of the minibody when expressed in the cell or cell line.

In some embodiments, the scFv, minibody, cys-diabody and/or antibody includes one or more of the residues in the humanized sequence shown in FIG. 2A and/or 2B and denoted with an asterisk. In some embodiments, while one or more of the residues marked with an asterisk in FIG. 2A or 2B is present; the remaining sequence can be varied. For example, the sequence can be 80, 81, 82, 83, 84, 85, 86, 87, 88, 89, 90, 91, 92, 93, 94, 95, 96, 97, 98, 99 percent or greater identity to the remaining sections of the sequence. In some embodiments, the human and/or humanized antigen binding construct will include one or more of the asterisked residues in FIG. 2A, for example 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, or 47. In some embodiments, the antigen binding construct includes one or more of the underlined residues in FIG. 2A. In some embodiments, the antigen binding construct includes one or more of the non-underlined residues in FIG. 2A. In some embodiments, the antigen binding construct includes one or more of the non-underlined residues in FIG. 2A as well as the boxed CDR sections, whereas other residues are allowed to vary. In some embodiments, the antigen binding construct

Alternatively, and/or in addition to, the antigen binding construct can include one or more of the asterisked residues in FIG. 2A or 2B, for example 1, 2, 3, 4, 5, 6, 7, 8, 9, 10, 11, 12, 13, 14, 15, 16, 17, 18, 19, or 20. In some embodiments, the antigen binding construct includes one or more of the non-underlined residues in FIG. 2A or 2B. In some embodiments, the antigen binding construct includes one or more of the non-underlined residues in FIG. 2A or 2B as well as the boxed CDR sections, whereas other residues are allowed to vary. In some embodiments, the CDR residues are maintained and the residues with the asterisk are maintained, but one or more of the other residues are allowed to vary.

In some embodiments. a chimeric minibody that binds to the target molecule is provided. In some embodiments, the chimeric minibody includes a monomer in the V_{L}-V_{H} format, and includes the sequence of SEQ ID NO:16 or 20, or a sequence having at least about 80% identity thereto, for example at least about 80%, 85%, 90%, 95%, 96%, 97%, 98%, or 99% % identity thereto. In some embodiments, the chimeric minibody includes a monomer in the V_{H}-V_{L} format, and includes the sequence of SEQ ID NO:18 or 22, or a sequence having at least about 80% identity thereto, for example at least about 80%, 85%, 90%, 95%, 96%, 97%, 98%, or 99% % identity thereto.

In some embodiments, the polypeptide of the monomer includes any of the combined sections as indicated in Tables 0.1 and 0.2, including polypeptides of the monomer with a sequences of at least about 80% identity, for example at least about 80%, 85%, 86%, 87%, 88%, 89%, 90%, 91%, 92%, 93, 94, 95%, 96%, 97%, 98%, 99%, or 100% identity to the monomers as set forth in Tables 0.1 and 0.2.

### Nucleic Acids

In some embodiments, the polypeptides of the antigen binding constructs can be encoded by nucleic acids and expressed in vivo or in vitro, or these peptide can be synthesized chemically. Thus, in some embodiments, a nucleic acid encoding an antigen binding construct is provided. In some embodiments, the nucleic acid encodes one part or monomer of a cys-diabody or minibody. In some embodiments, the nucleic acid encodes two or more monomers, for example, at least 2 monomers. Nucleic acids encoding multiple monomers can include nucleic acid cleavage sites between at least two monomers, can encode transcription or translation start site between two or more monomers, and/or can encode proteolytic target sites between two or more monomers.

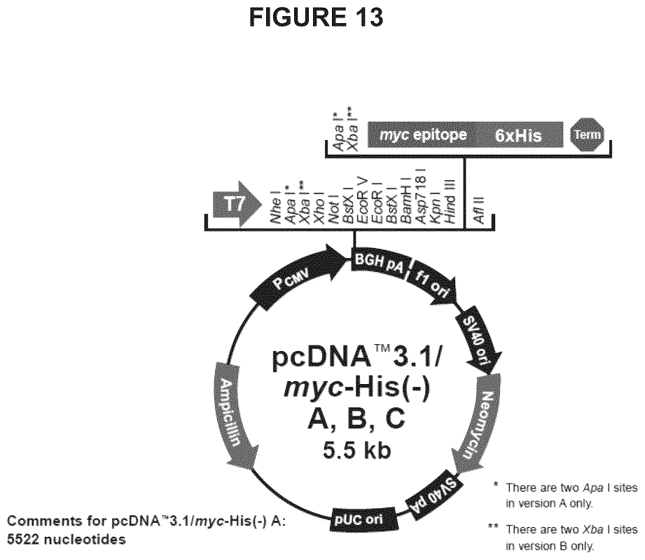

In some embodiments, an expression vector contains a nucleic acid encoding an antigen binding construct as disclosed herein. In some embodiments, the expression vector includes pcDNA3.1™/myc-His (-) Version A vector for mammalian expression (Invitrogen, Inc.), or a variant thereof (see FIG. 13). The pcDNA3.1 expression vector features a CMV promoter for mammalian expression and both mammalian (Neomycin) and bacterial (Ampicillin) selection markers (see FIG. 10). In some embodiments, the expression vector includes a plasmid. In some embodiments, the vector includes a viral vector, for example a retroviral or adenoviral vector. In embodiments, the vector includes a cosmid, YAC, or BAC.

In some embodiments, the nucleotide sequence encoding at least one of the minibody monomers comprises at least one of SEQ ID NOs:17, 19, 21, 23, 39, 41, 43, 45, 47, 49, 51, or a sequence having at least about 80% identity, for example about 80%, 85%, 86%, 87%, 88%, 89%, 90%, 91%, 92%, 93, 94, 95%, 96%. 97%, 98%, 99%, or greater identity thereto.

In some embodiments, the nucleotide sequence encoding at least one of the cys-diabody monomers includes SEQ ID NOs:77, 78, 10, 11, 39, 41, 43, 45, 47, 49, 51, or a sequence having at least about 80% identity, for example about 80%, 85%, 86%, 87%, 88%, 89%, 90%, 91%, 92%, 93, 94, 95%, 96%, 97%, 98%, 99% or greater identity thereto.

### Cell Lines

In some embodiments, a cell line is provided that expresses at least one of the antigen binding constructs described herein. In some embodiments, a mammalian cell line (e.g., CHO-K1 cell line) is an expression system to produce the minibodies, cys-diabodies or other antibodies as described herein. In some embodiments, the minibodies, cys-diabodies and other antibodies or antibody fragments described herein are non-glycosylated, and a mammalian expression system is not required, as such post-translational modifications are not needed. Thus, in some embodiments, one or more of a wide variety of mammalian or non-mammalian expression systems are used to produce the antigen binding constructs disclosed herein (for example., anti-CD8 minibodies and cys-diabodies) including, but not limited to mammalian expression systems (e.g., CHO-K1 cells), bacterial expression systems (e.g., E. Coli, B. subtilis) yeast expression systems (e.g., Pichia, S. cerevisiae) or any other known expression system. Other systems can include insect cells and/or plant cells.

### Antigen Binding Construct Modifications

In some embodiments, the antigen binding construct includes at least one modification. Exemplary modifications include, but are not limited to, antigen binding constructs that have been modified by glycosylation, acetylation, pegylation, phosphorylation, amidation, derivatization by known protecting/blocking groups, proteolytic cleavage, and linkage to a cellular ligand or other protein. Any of numerous chemical modifications may be carried out by known techniques, including, but not limited to, specific chemical cleavage, acetylation, formylation and metabolic synthesis of tunicamycin. In some embodiments, the derivative can contain one or more non-natural amino acids.

In some embodiments, the antigen binding construct is conjugated to another substance to form an anti-target conjugate. The conjugates described herein can be prepared by known methods of linking antigen binding constructs with lipids, carbohydrates, protein or other atoms and molecules. In some embodiments, the conjugate is formed by site-specific conjugation using a suitable linkage or bond. Site-specific conjugation is more likely to preserve the binding activity of an antigen binding construct. The substance may be conjugated or attached at the hinge region of a reduced antigen binding construct via disulfide bond formation. For example, introduction of cysteine residues at the C-terminus of a scFv fragment, such as those that can be introduced in the cys-diabodies described herein, allows site-specific thiol-reactive coupling at a site away from the antigen binding site to a wide variety of agents. Other linkages or bonds used to form the conjugate can include, but are not limited to, a covalent bond, a non-covalent bond, a sulfide linkage, a hydrazone linkage, a hydrazine linkage, an ester linkage, an amido linkage, and amino linkage, an imino linkage, a thiosemicabazone linkage, a emicarbazone linkage, an oxime linkage and a carbon-carbon linkage.

### Detectable Markers

In some embodiments, a modified antigen binding construct is conjugated to a detectable marker. As used herein, a “detectable marker” includes an atom, molecule, or compound that is useful in diagnosing, detecting or visualizing a location and/or quantity of a target molecule, cell, tissue, organ and the like. Detectable markers that can be used in accordance with the embodiments herein include, but are not limited to, radioactive substances (e.g., radioisotopes, radionuclides, radiolabels or radiotracers), dyes, contrast agents, fluorescent compounds or molecules, bioluminescent compounds or molecules, enzymes and enhancing agents (e.g., paramagnetic ions). In addition, some nanoparticles, for example quantum dots and metal nanoparticles (described below) can be suitable for use as a detection agent. In some embodiments, the detectable marker is IndoCyanine Green (ICG).

Exemplary radioactive substances that can be used as detectable markers in accordance with the embodiments herein include, but are not limited to, ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴-¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Exemplary Paramagnetic ions substances that can be used as detectable markers include, but are not limited to ions of transition and lanthanide metals (e.g. metals having atomic numbers of 6 to 9, 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

When the detectable marker is a radioactive metal or paramagnetic ion, in some embodiments, the marker can be reacted with a reagent having a long tail with one or more chelating groups attached to the long tail for binding these ions. The long tail can be a polymer such as a polylysine, polysaccharide, or other derivatized or derivatizable chain having pendant groups to which may be bound to a chelating group for binding the ions. Examples of chelating groups that may be used according to the embodiments herein include, but are not limited to, ethylenediaminetetraacetic acid (EDTA), diethylenetriaminepentaacetic acid (DTPA), DOTA, NOTA, NOGADA, NETA, deferoxamine (DfO), porphyrins, polyamines, crown ethers, bis-thiosemicarbazones, polyoximes, and like groups. The chelate can be linked to the antigen binding construct by a group which allows formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antigen binding constructs and carriers described herein. Macrocyclic chelates such as NOTA, NOGADA, DOTA, and TETA are of use with a variety of metals and radiometals including, but not limited to, radionuclides of gallium, yttrium and copper, respectively. Other ring-type chelates such as macrocyclic polyethers, which are of interest for stably binding radionuclides, such as Radium-223 for RAIT may be used. In certain embodiments, chelating moieties may be used to attach a PET imaging agent, such as an Aluminum-18F complex, to a targeting molecule for use in PET analysis.

Exemplary contrast agents that can be used as detectable markers in accordance with the embodiments of the disclosure include, but are not limited to, barium, diatrizoate, ethiodized oil, gallium citrate, iocarmic acid, iocetamic acid, iodamide, iodipamide, iodoxamic acid, iogulamide, iohexyl, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, thallous chloride, or combinations thereof.

Bioluminescent and fluorescent compounds or molecules and dyes that can be used as detectable markers in accordance with the embodiments of the disclosure include, but are not limited to, fluorescein, fluorescein isothiocyanate (FITC), OREGON GREEN™, rhodamine, Texas red, tetrarhodimine isothiocynate (TRITC), Cy3, Cy5, and the like), fluorescent markers (e.g., green fluorescent protein (GFP), phycoerythrin, and the like), autoquenched fluorescent compounds that are activated by tumor-associated proteases, enzymes (e.g., luciferase, horseradish peroxidase, alkaline phosphatase, and the like), nanoparticles, biotin, digoxigenin or combination thereof.

Enzymes that can be used as detectable markers in accordance with the embodiments of the disclosure include, but are not limited to, horseradish peroxidase, alkaline phosphatase, acid phosphatase, glucose oxidase, β-galactosidase, β-glucoronidase or β-lactamase. Such enzymes may be used in combination with a chromogen, a fluorogenic compound or a luminogenic compound to generate a detectable signal.

In some embodiments, the antigen binding construct is conjugated to a nanoparticle. The term “nanoparticle” refers to a microscopic particle whose size is measured in nanometers, e.g., a particle with at least one dimension less than about 100 nm. Nanoparticles can be used as detectable substances because they are small enough to scatter visible light rather than absorb it. For example, gold nanoparticles possess significant visible light extinction properties and appear deep red to black in solution. As a result, compositions comprising antigen binding constructs conjugated to nanoparticles can be used for the in vivo imaging of T-cells in a subject. At the small end of the size range, nanoparticles are often referred to as clusters. Metal, dielectric, and semiconductor nanoparticles have been formed, as well as hybrid structures (e.g. core-shell nanoparticles). Nanospheres, nanorods, and nanocups are just a few of the shapes that have been grown. Semiconductor quantum dots and nanocrystals are examples of additional types of nanoparticles. Such nanoscale particles, when conjugated to an antigen binding construct, can be used as imaging agents for the in vivo detection of T-cells as described herein.

### Therapeutic Agents

In some embodiments, an antigen binding construct is conjugated to a therapeutic agent. A “therapeutic agent” as used herein is an atom, molecule, or compound that is useful in the treatment of cancer, inflammation, other disease conditions, or to otherwise suppress an immune response, for example immunosuppression in organ transplants. Examples of therapeutic agents include, but are not limited to, drugs, chemotherapeutic agents, therapeutic antibodies and antibody fragments, toxins, radioisotopes, enzymes (e.g., enzymes to cleave prodrugs to a cytotoxic agent at the site of the antigen binding construct binding), nucleases, hormones, immunomodulators, antisense oligonucleotides, chelators, boron compounds, photoactive agents and dyes, and nanoparticles.

Chemotherapeutic agents are often cytotoxic or cytostatic in nature and may include alkylating agents, antimetabolites, anti-tumor antibiotics, topoisomerase inhibitors, mitotic inhibitors hormone therapy, targeted therapeutics and immunotherapeutics. In some embodiments the chemotherapeutic agents that may be used as detectable markers in accordance with the embodiments of the disclosure include, but are not limited to, 13-cis-Retinoic Acid, 2-Chlorodeoxyadenosine, 5-Azacitidine, 5-Fluorouracil, 6-Mercaptopurine, 6-Thioguanine, actinomycin-D, adriamycin, aldesleukin, alemtuzumab, alitretinoin, all-transretinoic acid, alpha interferon, altretamine, amethopterin, amifostine, anagrelide, anastrozole, arabinosylcytosine, arsenic trioxide, amsacrine, aminocamptothecin, aminoglutethimide, asparaginase, azacytidine, bacillus calmette-guerin (BCG), bendamustine, bevacizumab, bexarotene, bicalutamide, bortezomib, bleomycin, busulfan, calcium leucovorin, citrovorum factor, capecitabine, canertinib, carboplatin, carmustinc, cetuximab, chlorambucil, cisplatin, cladribine, cortisone, cyclophosphamide, cytarabine, darbepoetin alfa, dasatinib, daunomycin, decitabine, denileukin diftitox, dexamethasone, dexasone, dexrazoxane, dactinomycin, daunorubicin, decarbazine, docetaxel, doxorubicin, doxifluridine, eniluracil, epirubicin, epoetin alfa, erlotinib, everolimus, exemestane, estramustine, etoposide, filgrastim, fluoxymesterone, fulvestrant, flavopiridol, floxuridine, fludarabine, fluorouracil, flutamide, gefitinib, gemcitabine, gemtuzumab ozogamicin, goserelin, granulocyte-colony stimulating factor, granulocyte macrophage-colony stimulating factor, hexamethylmelamine, hydrocortisone hydroxyurca, ibritumomab, interferon alpha, interleukin-2, interleukin-11, isotretinoin, ixabepilone, idarubicin, imatinib mesylate, ifosfamide, irinotecan, lapatinib, lenalidomide, letrozole, leucovorin, leuprolide, liposomal Ara-C, lomustine, mechlorethamine, megestrol, melphalan, mercaptopurine, mesna, methotrexate, methylprednisolone, mitomycin C, mitotane, mitoxantrone, nelarabine, nilutamide, octreotide, oprelvekin, oxaliplatin, paclitaxel, pamidronate, pemetrexed, panitumumab, PEG Interferon, pegaspargase, pegfilgrastim, PEG-L-asparaginase, pentostatin, plicamycin, prednisolone, prednisone, procarbazine, raloxifene, rituximab, romiplostim, ralitrexed, sapacitabine, sargramostim, satraplatin, sorafenib, sunitinib, semustine, streptozocin, tamoxifen, tegafur, tegafur-uracil, temsirolimus, temozolamide, teniposide, thalidomide, thioguanine, thiotepa, topotecan, toremifene, tositumomab, trastuzumab, tretinoin, trimitrexate, alrubicin, vincristine, vinblastine, vindestine, vinorelbine, vorinostat, or zoledronic acid.

Toxins that may be used as detectable markers in accordance with the embodiments of the disclosure include, but are not limited to, ricin, abrin, ribonuclease (RNase), DNase I, Staphylococcal enterotoxin-A, pokeweed antiviral protein, gelonin, diphtheria toxin, Pseudomonas exotoxin, and Pseudomonas endotoxin.

In some embodiments nanoparticles are used in therapeutic applications as drug carriers that, when conjugated to an antigen binding construct, deliver chemotherapeutic agents, hormonal therapeutic agents, radiotherapeutic agents, toxins, or any other cytotoxic or anti-cancer agent known in the art to cancerous cells that overexpress the target on the cell surface.

Any of the antigen binding constructs described herein may be further conjugated with one or more additional therapeutic agents, detectable markers, nanoparticles, carriers or a combination thereof. For example, an antigen binding construct may be radiolabeled with Iodine 131 and conjugated to a lipid carrier, such that the anti-CD8-lipid conjugate forms a micelle. The micelle can incorporate one or more therapeutic or detectable markers. Alternatively, in addition, the antigen binding construct may be radiolabeled with lodine 131 (for example, at a tyrosine residue) and conjugated to a drug (for example, at the epsilon amino group of a lysine residue), and the carrier may incorporate an additional therapeutic or detectable marker.

### Kits

In some embodiments, kits are provided. In some embodiments, the kit includes an antigen binding construct as described herein. In some embodiments, the kit includes a nucleic acid that encodes an antigen binding construct as described herein. In some embodiments, the kit includes a cell line that produces an antigen binding construct as described herein. In some embodiments, the kit includes a detectable marker as described herein. In some embodiments, the kit includes a therapeutic agent as described herein. In some embodiments, the kit includes buffers. In some embodiments, the kit includes positive controls, for example CD8, CD8+ cells, or fragments thereof. In some embodiments, the kit includes negative controls, for example a surface or solution that is substantially free of CD8. In some embodiments, the kit includes packaging. In some embodiments, the kit includes instructions.

### Methods of Detecting the Presence or Absence of the Target Molecule

Antigen binding constructs can be used to detect the presence or absence of the target molecule in vivo and/or in vitro. Accordingly, some embodiments include methods of detecting the presence or absence of the target. The method can include applying an antigen binding construct to a sample. The method can include detecting a binding or an absence of binding of the antigen binding construct to the target molecule, CD8.

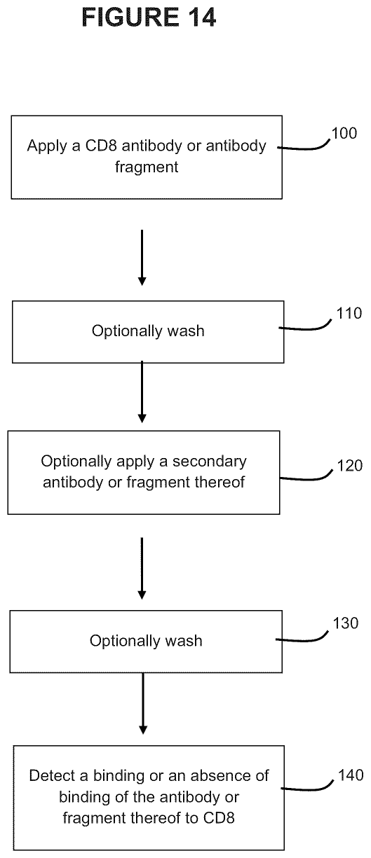

FIG. 14 illustrates some embodiments of methods of detecting the presence or absence of CD8. It will be appreciated that the steps shown in FIG. 14 can be performed in any sequence, and/or can be optionally repeated and/or eliminated, and that additional steps can optionally be added to the method. An antigen binding construct as described herein can be applied to a sample 100. An optional wash 110 can be performed. Optionally, a secondary antigen binding construct can be applied to the sample 120. An optional wash can be performed 130. A binding or absence of binding of the antigen binding construct to the target molecule can be detected 140.

In some embodiments, an antigen binding construct as described herein is applied to a sample in vivo. The antigen binding construct can be administered to a subject. In some embodiments, the subject is a human. In some embodiments, the subject is a non-human mammal, for example a rat, mouse, guinea pig, hamster, rabbit, dog, cat, cow, horse, goat, sheep, donkey, pig, monkey, or ape. In some embodiments, the antigen binding construct is infused into the subject. In some embodiments, the infusion is intravenous. In some embodiments, the infusion is intraperitoneal. In some embodiments, the antigen binding construct is applied topically or locally (as in the case of an interventional or intraoperative application) to the subject. In some embodiments, a capsule containing the antigen binding construct is applied to the subject, for example orally or intraperitoneally. In some embodiments, the antigen binding construct is selected to reduce the risk of an immunogenic response by subject. For example, for a human subject, the antigen binding construct can be humanized as described herein. In some embodiments, following in vivo application of the antigen binding construct, the sample, or a portion of the sample is removed from the host. In some embodiments, the antigen binding construct is applied in vivo, is incubated in vivo for a period of time as described herein, and a sample is removed for analysis in vitro, for example in vitro detection of antigen binding construct bound to the target molecule or the absence thereof as described herein.

In some embodiments, the antigen binding construct is applied to a sample in vitro. In some embodiments, the sample is freshly harvested from a subject, for example a biopsy. In some embodiments, the sample is incubated following harvesting from a subject. In some embodiments, the sample is fixed. In some embodiments the sample includes a whole organ and/or tissue. In some embodiments, the sample includes one or more whole cells. In some embodiments the sample is from cell extracts, for example lysates. In some embodiments, antigen binding construct in solution is added to a solution in the sample. In some embodiments, antigen binding construct in solution is added to a sample that does not contain a solution, for example a lyophilized sample, thus reconstituting the sample. In some embodiments, lyophilized antigen binding construct is added to a sample that contains solution, thus reconstituting the antigen binding construct.

In some embodiments, the antigen binding construct is optionally incubated with the sample. The antigen binding construct can be incubated for a period of no more than about 10 days, for example no more than about 10 days, 9, 8, 7, 6, 5, 4, 3, 2, or 1 day, or no more than about 23 hours, for example no more than about 23 hours, 22, 21, 20, 19, 18, 17, 16, 15, 14, 13, 12, 11, 10, 9, 8, 7, 6, 5, 4, 3, 2, 1, 0.75, 0.5, 0.25, or 0.1 hour, including ranges between any two of the listed values. In some embodiments, the incubation is within a subject to which the antigen binding construct was administered. In some embodiments, the incubation is within an incubator. In some embodiments, the incubator is maintained at a fixed temperature, for example about 21° C., room temperature, 25° C., 29° C., 34° C., 37° C. or 40° C.

In some embodiments, the antigen binding construct that is not bound to the target is optionally removed from the sample. In some embodiments, the sample is washed. Washing a sample can include removing solution that contains unbound antigen binding construct, and adding solution that does not contain antigen binding construct, for example buffer solution. In some embodiments, an in vitro sample is washed, for example by aspirating, pipetting, pumping, or draining solution that contains unbound antigen binding construct, and adding solution that does not contain antigen binding construct. In some embodiments, an in vivo sample is washed, for example by administering to the subject solution that does not contain antigen binding construct, or by washing a site of topical antigen binding construct administration. In some embodiments, the wash is performed at least two times, for example at least 2, 3, 4, 5, 6, 7, 8, 9, 10, 15, or 20 times. In some embodiments, following the wash or washes, at least about 50% of unbound antibody is removed from the sample, for example at least about 50%, 60%, 70%, 75%, 80%, 85%, 90%, 95%, 97%, 98%, 99% or greater.

In some embodiments, unbound antigen binding construct is eliminated from the sample. Following application of the antigen binding construct to the sample, antigen binding construct bound to the target reaches an equilibrium with antigen binding construct unbound to the target, so that at some time after application of the antigen binding construct, the amount of antigen binding construct bound to the target does not substantially increase. After this time, at least part of the quantity of the antigen binding construct that is unbound to the target can be eliminated. In some embodiments, unbound antigen binding construct is eliminated by metabolic or other bodily processes of the subject to whom the antibody or fragment was delivered. In some embodiments, unbound antigen binding construct is eliminated by the addition of an agent that destroys or destabilized the unbound antigen binding construct, for example a protease or a neutralizing antibody. In some embodiments, 1 day after application of the antigen binding construct, at least about 30% of the antigen binding construct that was applied has been eliminated, for example at least about 30%, 40%, 60%, 70%, 75%, 80%, 85%, 90%, 95%, 97%, 98%, 99%, or 99.9%. In some embodiments, 2 days after application of the antigen binding construct, at least about 40% of the antigen binding construct that was applied has been eliminated, for example at least about 40%, 50%, 60%, 70%, 75%, 80%, 85%, 90%, 95%, 97%, 98%, 99%, or 99.9%.

In some embodiments, the presence or absence of the target, CD8, is detected. The presence or absence of the target can be detected based on the presence or absence of the antigen binding construct in the sample. After removal and/or elimination of the antigen binding construct from the sample, for example by washing and/or metabolic elimination, remaining antigen binding construct in the sample can indicate the presence of the target, while an absence of the antigen binding construct in the sample can indicate the absence of the target.

In some embodiments, the antigen binding construct includes a detectable marker as described herein. Thus, the presence of the antigen binding construct can be inferred by detecting the detectable marker.

In some embodiments, a secondary antigen binding construct is used to detect the antigen binding construct. The secondary antigen binding construct can bind specifically to the antigen binding construct. For example, the secondary antigen binding construct can include a polyclonal or monoclonal antibody, diabody, minibody, etc. against the host type of the antibody, or against the antigen binding construct itself. The secondary antigen binding construct can be conjugated to a detectable marker as described herein. The secondary antigen binding construct can be applied to the sample. In some embodiments, the secondary antigen binding construct is applied to the sample in substantially the same manner as the antigen binding construct. For example, if the antigen binding construct was infused into a subject, the secondary antigen binding construct can also be infused into the subject.

In some embodiments, binding or the absence of binding of the antigen binding construct is detected via at least one of: positron emission tomography (PET), single-photon emission computed tomography (SPECT), magnetic resonance imaging (NMR), or detection of fluorescence emissions. PET can include, but is not limited to microPET imaging. In some embodiments, binding of the absence of binding of the antigen binding construct is detected via two or more forms of imaging. In some embodiments, detection can be via near-infrared (NIR) and/or Cerenkov.

### Methods of Targeting a Therapeutic Agent to a Cell

Antigen binding constructs can be used to target a therapeutic molecule, for example a cytotoxin to a target positive cell, such as a cell expressing CD8. Thus, some embodiments include methods of targeting a therapeutic agent to a target positive cell. The method can include administering an antigen binding construct as described herein to a subject. The subject can be a subject in need, for example a subject in need of elimination or neutralization of at least some target positive cells. In some embodiments, the antigen binding construct includes at least on therapeutic agent as described herein. In some embodiments, the therapeutic can be directly conjugated to the antigen binding construct via a covalent bond, such as a disulfide bond. In some embodiments, the subject can benefit from the localization of a CD8 positive cell to another cell or agent.

Optionally, before and/or after administration of the antigen binding construct that includes at least one therapeutic agent, the number and/or localization of the target positive cells of the patient is determined. For example, determining the number and/or localization of target positive cells prior to administration can indicate whether the patient is likely to benefit from neutralization and/or elimination of the target positive cells. Determining the number and/or localization of the target positive cells after administration can indicate whether the target positive cells were eliminated in the patient.

### Additional Embodiments

Some embodiments include detection of human CD8 which is a specific biomarker found on the surface of a subset of T-cells for diagnostic imaging of the immune system. Imaging of the target molecule can allow for the in vivo detection of T-cell localization. Changes in T-cell localization can reflect the progression of an immune response and can occur over time as a result various therapeutic treatments or even disease states. For example, imaging T-cell localization can be useful in immunotherapy. Adoptive immunotherapy is a form of therapy where a patient's own T-cells are manipulated in vitro and re-introduced into the patient. For this form of treatment, imaging of T-cells can be useful for monitoring and/or determining the status of the treatment. Thus, in some embodiments, monitoring the localization of the target molecule can be a useful for analyzing a mechanism of action, efficacy, and/or safety in the development of drugs and/or can aid in the clinical management of disease.

In some embodiments, the CDRs of an antigen binding construct that binds specifically to a target, for example for the antibody OKT8, have been adjusted to minibody and cys-diabody arrangements. The CDRs of a murine antibody have been grafted onto a human minibody and cys-diabody framework, thus producing a chimeric minibody. Antibody V domains typically contain two cysteines that form intra-disulfide bonds. The OKT8 Vu has an extra cysteine in framework 3 (FR3) which could interfere with the expression of the protein as it may lead to aggregation and consequently retention in the endoplasmic reticulum. Thus, some embodiments include minibodies made with a serine replacing the extra cysteine in the framework (see, for example, SEQ ID:16, 18, 20, and 22).

In some embodiments, a method of targeting a CD8+ cell to a first antigen is provided. The method can include applying a bispecific antigen binding construct to a sample. The bispecific antigen binding construct can include a CD8 antigen binding construct as described herein. The bispecific antibody can include an antigen binding construct that binds to the first antigen, for example 1, 2, 3, 4, 5, or 6 CDR's, an scFv, or a monomer of a minibody or cys-diabody. In some embodiments, the bispecific antibody includes 1, 2, or 3 HCDR's of an antigen binding construct as described herein, and/or 1, 2, or 3 LCDR's of an antigen binding construct as described herein. In some embodiments, the bispecific antigen binding construct includes an scFv of an antigen binding construct as described herein. In some embodiment, the bispecific antigen binding construct includes a V_{H} or V_{L} sequence as described herein. In some embodiments, the bispecific an antigen binding construct includes a minibody or cys-diabody monomer as described herein. In some embodiments, the bispecific an antigen binding construct is applied to a sample in vivo, for example an organ or tissue of a subject. In some embodiments, the bispecific an antigen binding construct is applied to an in vitro sample. Without being limited to any one theory, in some embodiments, the bispecific an antigen binding construct binds to the target on the target positive cell, and binds to the first antigen (which can be different from CD8) on the first cell, and thus brings the target positive cell in proximity to the first cell. For example, a CD8+ T cell can be brought into proximity of a cancer cell, and can facilitate an immune response against that cancer cell.

In some embodiments, the anti-CD8 antigen binding constructs can be imaging agents that specifically target human CD8+ T-cells. In some embodiments, the anti-CD8 fragments can directly bind and detect the localization of the specific subclass of T-cells that express CD8. In some embodiments, engineered fragments able to cross link CD8 can potentiate signaling through the T cell receptor and enhance the ability of a subject to clear viral pathogens and respond to tumor antigens and vaccines.

In some embodiments, the minibody and cys-diabody antibody formats have desired pharmacokinetic characteristics for diagnostic imaging while maintaining the high binding affinity and specificity of the parental antibody. Compared to imaging with the full-length parental antibody, these fragments clear much faster; yet they are able to target the antigen for rapid high-contrast imaging. The same favorable pharmacokinetic properties are advantageous for targeting immune responses allowing for more controlled T cell stimulation and preventing undesirable effects of overstimulation (for example, cytokine storms). In preclinical models, the shorter serum half lives for the minibody and the cys-diabody allow for optimal imaging at approximately 16-20 hours post injection for the minibody and 2-6 hours post-injection for the cys-diabody. Same day imaging can provide a significant advantage in the clinic with respect to patient care management.

In addition, the cys-diabody antibody format features the C-terminus cysteine tail. These two sulfhydryl groups (following mild reduction) provide a strategy for site-specific conjugation of functional moieties such as radiolabels that will not interfere with the cys-diabody's binding activity.

In some embodiments, these antigen binding constructs can be diagnostic imaging agents (following labeling with an appropriate radioisotope such as lodine-124, Cu-64 or Zr-89 (for PET imaging) or fluorophore (for fluorescent imaging)). As clinical imaging agents, these CD8 antigen binding constructs can help to monitor treatment and be used as a patient selection tool.

In some embodiments, the antigen binding constructs can be used for applications where highly specific and high-affinity binding to CD8 is required. Outside of diagnostic imaging, these fragments could serve different purposes depending on the attachment of different functional groups.

With the attachment of the appropriate infrared or fluorescent dye, these constructs can be used as the targeting agent for image-guided intraoperative surgery.

In some embodiments, in addition to the modifications to the functional groups attached to the fragments, through the use of bispecific fragments (where the fragment is able to bind 2 different antigens) it is possible to bring together CD8+ cells to a second antigen. Bispecific full-length antibodies have been used in cancer immunotherapy to bring cytotoxic cells of the immune system to tumor cells. Thus, such embodiments are also contemplated for the appropriate antigen binding constructs.

In some embodiments, provided herein are engineered scFv, minibody, and cys-diabody antibody fragments that are able to bind and specifically target human CD8 alpha both in vitro and in vivo.

### Embodiments

The following embodiments are described herein:

1. An antigen binding construct that comprises:
a HCDR1 of a HCDR1 of SEQ ID NO:3 or 6;
a HCDR2 of HCDR1 of SEQ ID NO:3 or 6;
a HCDR3 of HCDR1 of SEQ ID NO:3 or 6;
a LCDR1 of LCDR1 of SEQ ID NO:9;
a LCDR2 of LCDR1 of SEQ ID NO:9; and
a LCDR3 of LCDR1 of SEQ ID NO:9.

2. The antigen binding construct of embodiment 1, wherein the antigen binding construct binds specifically to CD8.

3. The antigen binding construct of embodiment 1, further comprising a detectable marker.

4. The antigen binding construct of embodiment 1, further comprising a therapeutic agent.

5. The antigen binding construct of embodiment 1, wherein the antigen binding construct is bispecific.

6. The antigen binding construct of embodiment 1, wherein the antigen binding construct is a monovalent scFv.

7. A humanized cys-diabody that binds to CD8, the humanized cys-diabody comprising a polypeptide that comprises:
a single-chain variable fragment (scFv) comprising a variable heavy (VH) domain linked to a variable light (VL) domain; and
a C-terminal cysteine.

8. The humanized cys-diabody of embodiment 7, wherein the order of the variable domains, from N terminus to C terminus of the polypeptide is VL, VH.

9. The humanized cys-diabody of embodiment 7, wherein the order of the variable domains, from N terminus to C terminus of the polypeptide is VH, VL.

10. The humanized cys-diabody of embodiment 7, further comprising a detectable molecule.

11. The humanized cys-diabody of embodiment 7, wherein the humanized cys-diabody comprises:
a HCDR1 of a HCDR1 of SEQ ID NO:3 or 6;
a HCDR2 of HCDR1 of SEQ ID NO:3 or 6;
a HCDR3 of HCDR1 of SEQ ID NO:3 or 6;
a LCDR1 of LCDR1 of SEQ ID NO:9;
a LCDR2 of LCDR1 of SEQ ID NO:9; and
a LCDR3 of LCDR1 of SEQ ID NO:9.

12. A humanized minibody that binds to CD8, the humanized minibody comprising a polypeptide that comprises from N-terminus to C-terminus:
a single-chain variable fragment (scFv) that binds to CD8, the scFv comprising a variable heavy (VH) domain linked to a variable light (VL) domain;
a hinge-extension domain comprising a human IgGI hinge region; and
a human IgG CH3 sequence.

13. The humanized minibody of embodiment 12, further comprising a detectable marker.

14. The humanized minibody of embodiment 12, wherein the humanized minibody comprises:
a HCDR1 of a HCDR1 of SEQ ID NO:3 or 6;
a HCDR2 of HCDR1 of SEQ ID NO:3 or 6;
a HCDR3 of HCDR1 of SEQ ID NO:3 or 6;
a LCDR1 of LCDR1 of SEQ ID NO:9;
a LCDR2 of LCDR1 of SEQ ID NO:9; and
a LCDR3 of LCDR1 of SEQ ID NO:9.

15. A nucleic acid encoding an antibody of any one of embodiments 1-14.

16. A cell line producing an antibody of any of embodiments 1-14.

17. A kit comprising:
at least one of an antigen binding construct thereof of any of embodiments 1-14, a nucleic acid of embodiment 15, or a cell of a cell line of embodiment 16; and
a detectable marker.

18. A method of detecting a presence or absence of a CD8, the method comprising:
applying the antigen binding construct of any one of embodiments 1-14 to a sample; and
detecting a presence or an absence of the antigen binding construct, thereby detecting a presence of absence of a CD8.

19. The method of embodiment 18, wherein the antigen binding construct is conjugated to a detectable marker.

20. The method of embodiment 18, wherein applying the antigen binding construct comprises administering the antigen binding construct to a subject.

21. The method of embodiment 18, wherein detecting binding or absence of binding of the antigen binding construct to CD8 comprises at least one of positron emission tomography or single-photon emission computed tomography.

22. The method of embodiment 18, the method further comprising applying a secondary antigen binding construct to the sample, wherein the secondary antigen binding construct binds specifically to the antigen binding construct.

23. The method of embodiment 18, wherein the antigen binding construct is incubated with the sample for no more than 20 hours.

24. The method of embodiment 18, wherein the antigen binding construct is incubated with the sample for no more than 6 hours.

25. The method of embodiment 18, wherein the antigen binding construct is administered to a host, and wherein a first quantity of antigen binding construct thereof is unbound to CD8, and a second quantity of antigen binding construct is bound to CD8, wherein at least about 80% of the first quantity of antigen binding construct is eliminated in no more than 12 hours.

26. A method of targeting a therapeutic agent to a CD8, the method comprising administering to a subject an antigen binding construct of any one of embodiments 1-14, wherein the antigen binding construct is conjugated to a therapeutic agent.

### EXAMPLE 1: HUMANIZATION OF CD8 ANTIBODIES AND ANTIBODY FRAGMENTS

The murine variable regions of the OKT8 antibody were humanized by grafting the murine Complimentary Determining Region (CDR) onto a human framework. The murine V genes were run against the human V germ-line database. The human V gene with highest sequence homology was examined for functional residues and similarity to antigen binding loop (CDRs) structures. The V_{L} and V_{H} CDRs of the murine OKT8 were then incorporated into the human acceptor variable region framework, replacing the human CDRs. An alignment of the corresponding murine, human germline, and humanized sequences is shown for the heavy chain variable regions (FIG. 2A) and light chain variable regions (FIG. 2B). In these figures, the CDRs are boxed and the asterisks indicate residues that differ from each other. Selected mouse residues, known to function in the loop structure, were kept in the human framework.

### EXAMPLE 2: EXPRESSION OF CD8 MINIBODIES

The OKT8 minibody constructs (sequence combinations as outlined in Table 0.1) were transiently transfected into CHO-K1 cells to validate expression. The transfections were performed in a 6-well plate using the Lipofectamine reagent (Invitrogen). Following a 72-hour incubation at 37° C. in a CO2 incubator, the supernatants were harvested and filtered to remove any cells.

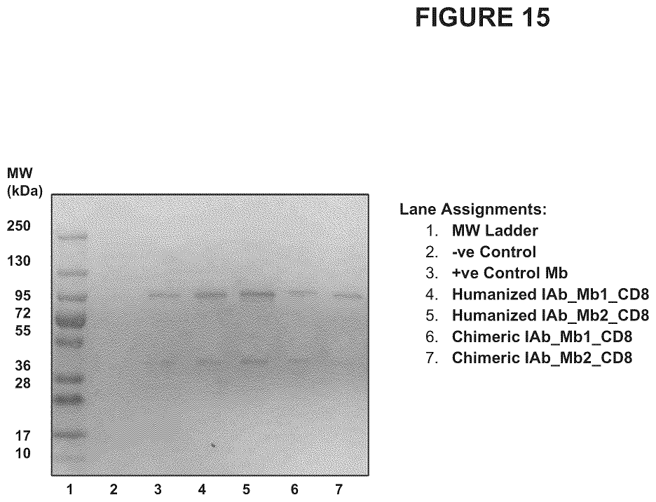

Western blot analysis was performed on supernatant from the transient transfections to confirm the expression of the antibody fragments. Supernatant from an empty vector transfection was included as a negative control, and supernatant from the transfection of an irrelevant minibody was used as a positive control. Under non-reducing conditions, the minibodies run at the expected molecular weight of 80-90 kDa (FIG. 15). A minor band representing the monomeric form is also detected at approximately 40 kDa. These results confirm the proper expression of the chimeric and humanized minibodies.

### EXAMPLE 3 BINDING

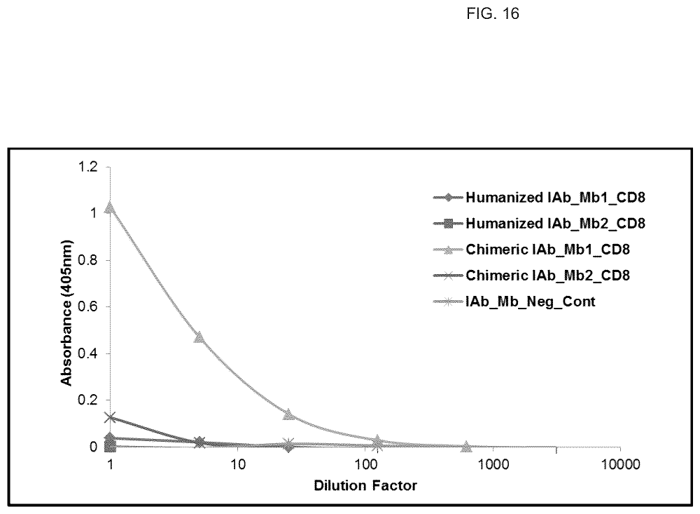

The chimeric minibody variant 1 demonstrated highest binding to CD8 by ELISA whereas the humanized variants did not show any significant binding to CD8 (FIG. 16). Although SPR analysis of the humanized variants showed binding to soluble CD8, several-fold loss in affinity was observed compared to chimeric minibody and parental OKT8 antibody. 96 well plates were coated with the recombinant human CD8 antigen and incubated with supernatants obtained from the transient transfection. Binding was detected with horseradish peroxidase (HRP) conjugated goat anti-human IgG (Fc-specific) and the chromogenic substrate 3,3′,5,5′-Tetramethylbenzidine (TMB) measuring the absorbance at 405nm. Dilutions were done in triplicate. Data is shown as mean of relative absorbance.

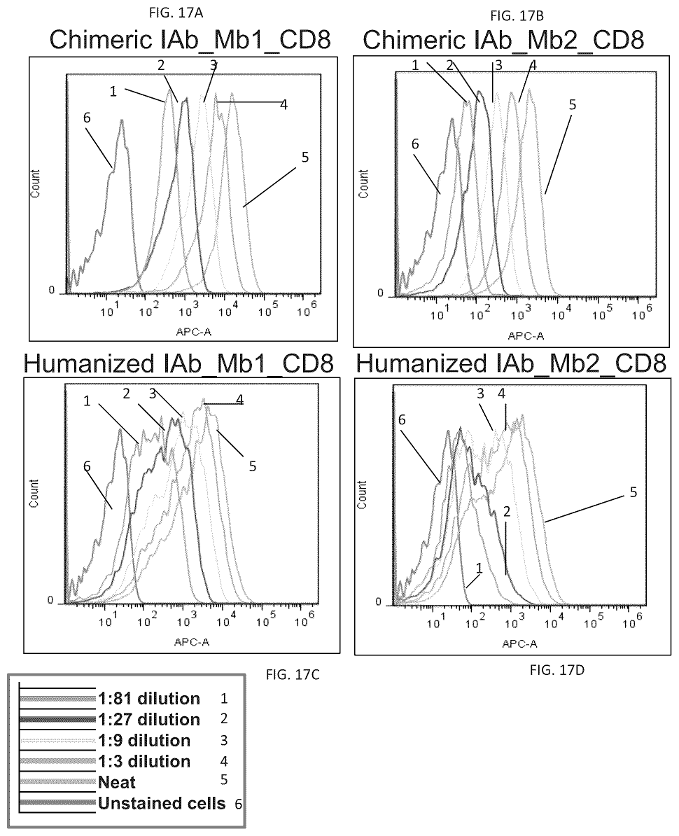

FIGS. 17A-17D display the results of the flow cytometry analysis of the IAb_Mb_CD8 variants. All histograms show allophycocyanin (APC) signal vs. counts. Supernatants from transfection of the variants at different dilutions were incubated with CD8+ cells. Cells were washed and subsequently stained with a secondary anti-human IgG (Fc-specific) APC conjugated antibody. 1×10⁵ cells were stained per point and analysis was performed with 10,000 events/point.

The chimeric and humanized minibodies showed concentration-dependent binding to the CD8+ cells (FIGS. 17A-17D). Although the humanized minibodies expressed better than the chimeric, the chimeric minibodies showed a stronger signal by the flow cytometry suggesting the chimeric minibodies possessed stronger binding affinity.

### EXAMPLE 4 MATURATION OF ANTIGEN BINDING CONSTRUCTS

To improve binding affinity of the humanized antibody fragments, the two humanized V_{H} regions were further affinity matured. For the 1^{st} version V_{H}, the affinity maturation resulted in sub-versions a and b. For the 2^{nd} version V_{H}, the affinity maturation resulted in sub-versions c and d. FIGS. 12F-12I display the resulting antibody variable light (V_{L}) and variable heavy (V_{H}) genes. The DNA with the amino acid sequences are shown. CDRs are boxed using Chothia definition.

The affinity matured humanized OKT8 V genes were engineered into two minibody variants that differed in the orientation of the V genes in the scFv; the V_{L}-V_{H} orientation referred to as number 1) and V_{H}-V_{L} orientation referred to as number 2. The specific sequence combinations are outlined in Table 0.2.

### EXAMPLE 5: EXPRESSION OF CD8 MINIBODIES

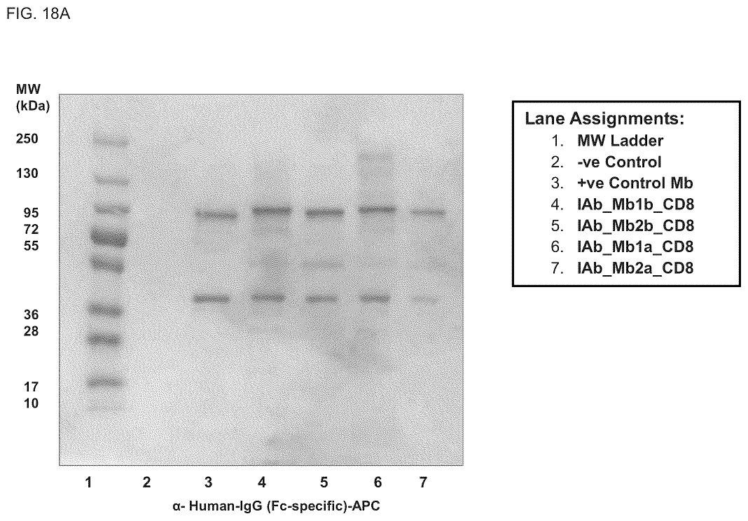

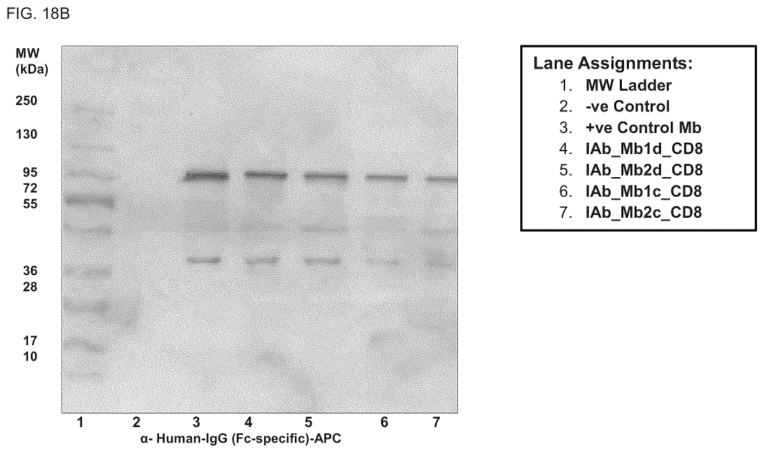

The above IAb_Mb_CD8 expression constructs were transiently transfected into CHO-K1 cells. Supernatant from the transfections was analyzed by Western blot to confirm proper expression of the minibodies. Supernatant from an empty vector transfection was included as a negative control, and purified protein of an irrelevant minibody was used as a positive control. All variants were expressed as evidenced by a band at the expected molecular weight for the assembled minibody complex (˜95 kDa) (FIG. 18A and FIG. 18B). The band present at ˜45 kDa represents the monomer. Transfection supernatants from transient transfectants were run on SDS-PAGE and transferred to PVDF membrane. The membrane was probed with alkaline phosphatase (AP)-conjugated anti-human IgG (Fc-specific) antibody and developed by incubating with the AP substrate BCIP/NBT. This is a representative blot of multiple experiments.

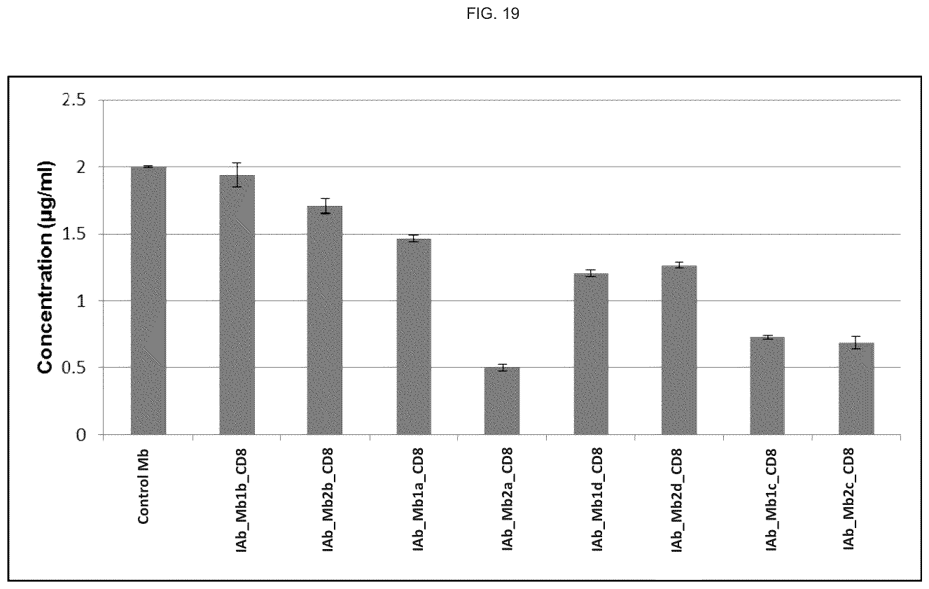

Quantitative ELISAs were performed to measure IAb_Mb_CD8 variants expression levels from the transient transfection in CHO-K1 cells. The IAb_Mb_CD8 minibody variants were expressed in a range between ˜0.5-1.9 ug/ml, with the higher end of the range being comparable with a previously expressed reference control minibody (FIG. 19). A goat anti-human IgG (Fc specific) was used to capture the minibody and an AP-conjugated goat anti-human IgG (Fc specific) was used for detection. Purified irrelevant isotype control minibody protein was used as a standard. IAb_Mb_CD8 supernatants were serially diluted to find dilution points which fit in the linear range of the standard curve.

### EXAMPLE 6: FUNCTIONAL ACTIVITY OF MINIBODIES

To demonstrate the functional activity of the IAb_Mb_CD8 minibody variants, the supernatants from the transient transfection were tested for binding to purified recombinant human CD8 protein by ELISA. The concentration of the variants IAb_Mb1b_CD8. IAb_Mb2b_CD8, and IAb_Mb1a_CD8 were normalized to match the concentration of IAb_MB2a_CD8 (0.5 μg/ml) based on the quantitative ELISA presented in FIG. 19. Samples were then serially diluted to assess binding over a series of concentrations. The parental OKT8 antibody was also included as a positive control for the assay (data not shown). All minibody variants showed concentration dependent binding to soluble recombinant human CD8 (rhCD8).

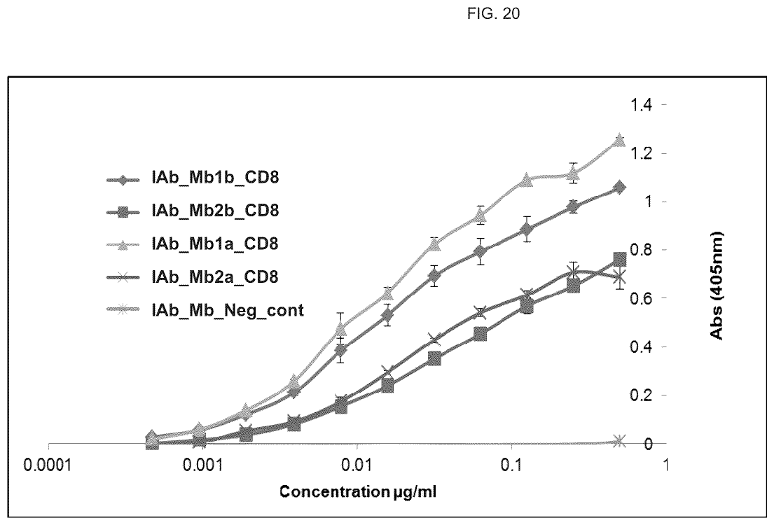

FIG. 20 indicates that IAb_Mb1a_CD8 followed by IAb_Mb1b_CD8 has the highest level of binding to the antigen. 96 well plates were coated with rhCDS antigen and incubated with supernatants obtained from the transient transfection. Binding was detected with HRP-conjugated goat anti-human (Fc-specific) IgG and TMB substrate. The absorbance measured at 405 nm. Dilutions were done in triplicate. Data is shown as mean of relative absorbance.

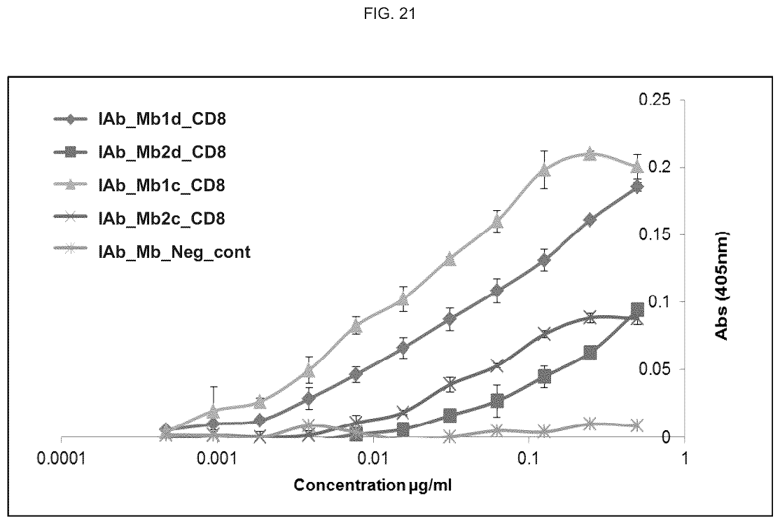

FIG. 21 indicated that variant Ic has the highest level of binding followed by variant 1c. 96 well plates were coated with rhCD8 antigen and incubated with supernatants obtained from the transient transfection. Binding was detected with HRP-conjugated goat anti-human (Fc-specific) IgG and TMB substrate. The absorbance measured at 405 nm. Dilutions were done in triplicate. Data is shown as mean of relative absorbance.

### EXAMPLE 7: BINDING TO CELLULAR HUMAN CD8

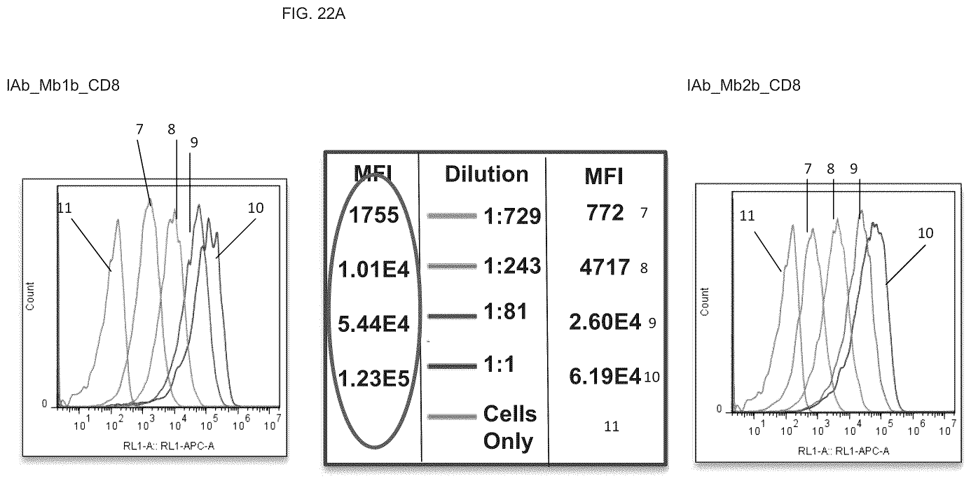

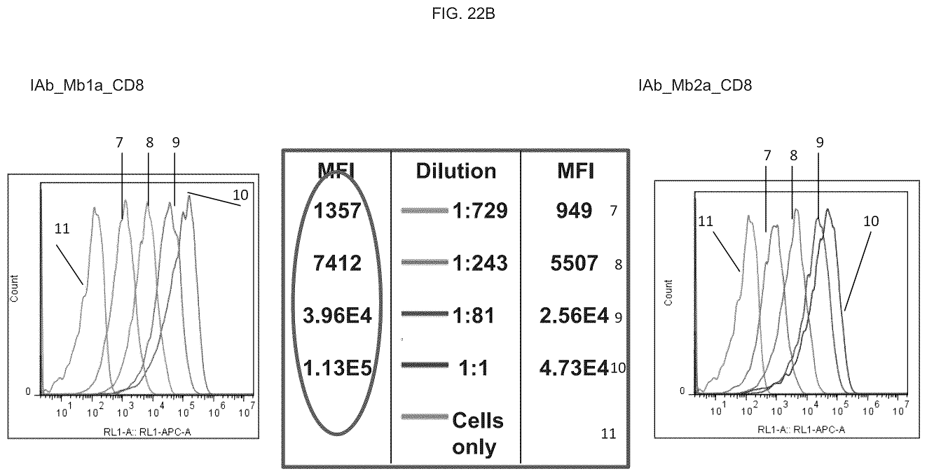

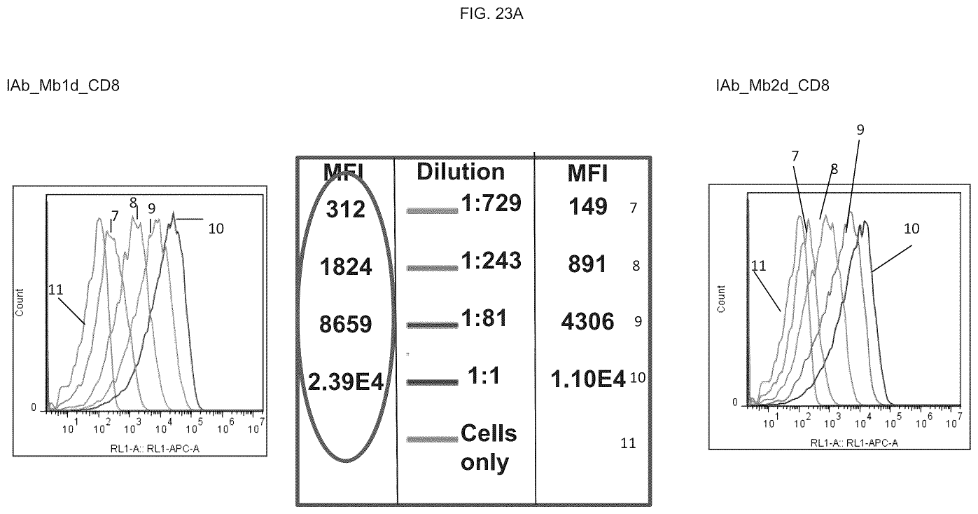

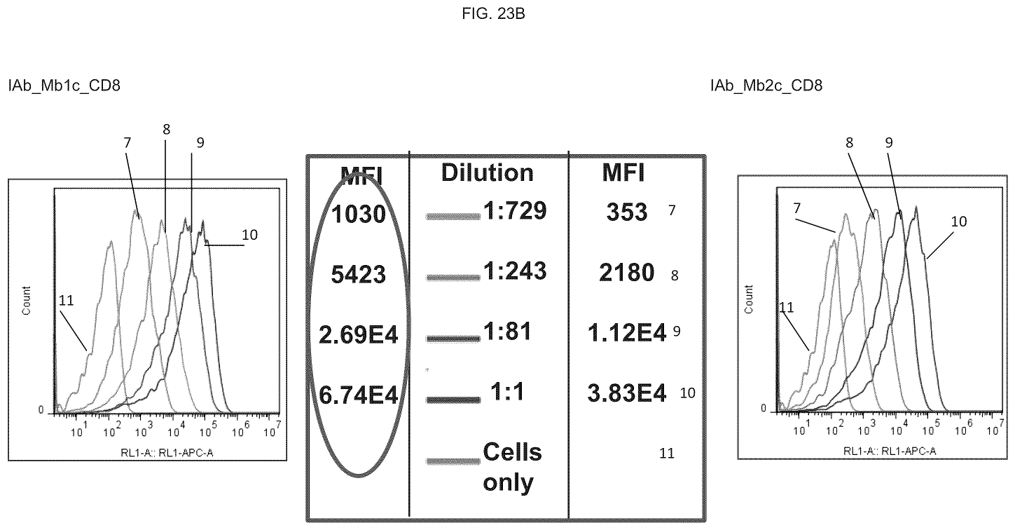

IAb_Mb_CD8 variants were evaluated for binding to cellular human CD8 using flow cytometry. Supernatants from the transient transfection were tested for binding to PC3-CD8 cells (PC3 cells stably transfected with human CD8) (FIGS. 22A and 22B and 23A and 23B). The minibody supernatants were normalized for the flow cytometry experiment. The parental OKTS was included as a positive control for binding (data not shown). PC3 cells was used as negative control and confirmed that the minibody variants did not bind (data not shown). IAb_Mb1b_CD8 demonstrated the highest Mean Fluorescence Intensity (MFI) of the four minibody variants.

In regard to the results presented in FIGS. 22A and 22B, all histograms show APC signal vs. counts. Supernatants from transfection of the variants were incubated with PC3-CD8 cells. Cells were washed and subsequently stained with APC-conjugated anti-human IgG (Fc-specific) antibody. 1×10⁵ cells were stained per point and analysis was performed with 10,000 events/point.

In regard to the results presented in FIGS. 23A and 23B, all histograms show APC signal vs. counts. Supernatants from transfection of the variants were incubated with PC3-CD8 cells. Cells were washed and subsequently stained with APC-conjugated anti-human IgG (Fc-specific) antibody. 1×10⁵ cells were stained per point and analysis was performed with 10,000 events/point.

The results indicated that the constructs still bind to cellular human CD8.

### EXAMPLE 8: SPR ANALYSIS

Surface plasma resonance (SPR) was used to determine the binding affinity for all the IAb_Mb_CD8 variants to recombinant human CD8 (Table 8.0). The minibody protein in the supernatant was captured on to the BIAcore chip using an anti-human IgG (Fc-specific) antibody. The amount of minibody captured on the chip was normalized to enable a direct comparison of binding affinity between the variants as a kinetic “scouting” experiment to rank affinities. The rhCD8 protein was passed over the captured minibody protein to measure binding. All variants showed strong binding to CD8 protein that was similar to the parental OKT8 mAb.

TABLE 8.0
Table 8.0 summarizes the measured association (ka), dissociation (kd), and kD
constants for the IAb_Mb_CD8 variants binding to recombinant hCD8. The variants were
captured on a BIAcore chip using anti-human Fc-specific IgG antibody.

| Ligand | Analyte | ka (1/Ms) | kd (1/s) | Conc of analyte | KA (1/M) | KD (M) |
|---|---|---|---|---|---|---|
| OKT8 mAb | hCD8 | 5.6 × 10⁵ | 1.0 × 10⁻³ | 50 nM | 5.5 × 10⁸ | 1.8 × 10⁻⁹ |
| IAb_Mb1b_CD8 | hCD8 | 6.4 × 10⁵ | 1.2 × 10⁻³ | 50 nM | 5.4 × 10⁸ | 1.9 × 10⁻⁹ |
| IAb_Mb2b_CD8 | hCD8 | 5.4 × 10⁵ | 2.1 × 10⁻³ | 50 nM | 2.6 × 10⁸ | 3.9 × 10⁻⁹ |
| IAb_Mb1a_CD8 | hCD8 | 6.5 × 10⁵ | 1.2 × 10⁻³ | 50 nM | 5.3 × 10⁸ | 1.9 × 10⁻⁹ |
| IAb_Mb2a_CD8 | hCD8 | 4.8 × 10⁵ | 2.2 × 10⁻³ | 50 nM | 2.2 × 10⁸ | 4.6 × 10⁻⁹ |
| IAb_Mb1d_CD8 | hCD8 | 7.0 × 10⁵ | 2.0 × 10⁻³ | 50 nM | 3.4 × 10⁸ | 2.9 × 10⁻⁹ |
| IAb_Mb2d_CD8 | hCD8 | 5.4 × 10⁵ | 2.9 × 10⁻³ | 50 nM | 1.8 × 10⁸ | 5.5 × 10⁻⁹ |
| IAb_Mb1c_CD8 | hCD8 | 7.0 × 10⁵ | 1.8 × 10⁻³ | 50 nM | 4.0 × 10⁸ | 2.5 × 10⁻⁹ |
| IAb_Mb2c_CD8 | hCD8 | 7.2 × 10⁵ | 3.2 × 10⁻³ | 50 nM | 2.2 × 10⁸ | 4.5 × 10⁻⁹ |

### EXAMPLE 9: EXPRESSION OF CD8 CYS-DIABODIES

The cys-diabody constructs as outlined in Table 0.3 (humanized version b) were transiently transfected into CHO-K1 cells to validate expression. The transfections were performed in a 6-well plate using the Lipofectamine reagent. Following a 72-hour incubation at 37° C. in a CO₂ incubator, the supernatants were harvested and filtered to remove any cells.

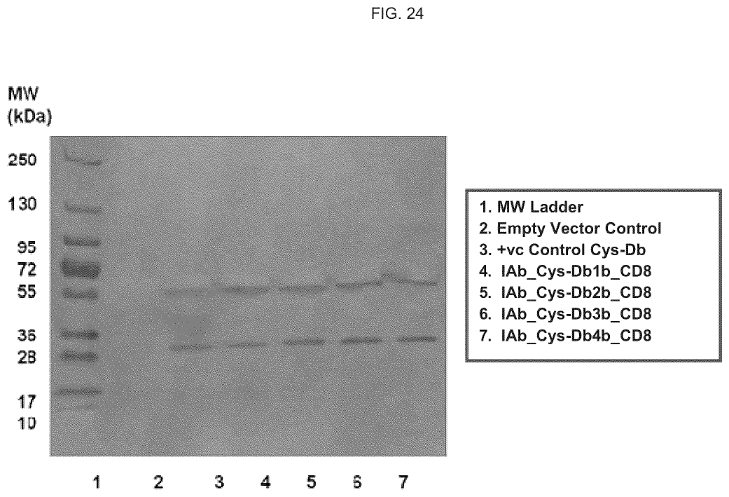

A western blot analysis was performed using supernatant from the transient transfections to confirm the expression of the antibody fragments. Supernatant from an empty vector transfection was included as a negative control, and supernatant from the transfection of an irrelevant cys-diabody was used as a positive control. Under non-reducing conditions, all four variants of the humanized OKT8 cys-diabody ran at the appropriate molecular weight of approximately 55 kD (FIG. 24). A minor band representing the monomeric form is also detected at approximately 25 kD. These results confirm the proper expression of the humanized OKT8 cys-diabodies. For the western blot, supernatants were collected following transient transfection into CHO-K1 cells. Transfection supernatants were electrophoresed by SDS-PAGE under non-reducing conditions and transferred to a PVDF membrane. The membrane was probed with an HRP-conjugated anti-His antibody and developed with the HRP substrate TMB.

### EXAMPLE 10: BINDING FOR CD8 CYS-DIABODIES

The supernatants from the transient transfection of the IAb_Cys-Dbb_CD8 variants were tested for binding to recombinant human CD8 (rhCD8) protein by ELISA. Samples were serially diluted to assess binding over a range of concentrations prior to incubation with CD8. 96 well plates were coated with rhCD8 antigen. Coated plates were incubated with supernatants following transient transfection of the different cys-diabody variants, and then incubated with HRP-conjugated anti-His antibody. The signal was detected using TMB and absorbance measured at 405 nm.

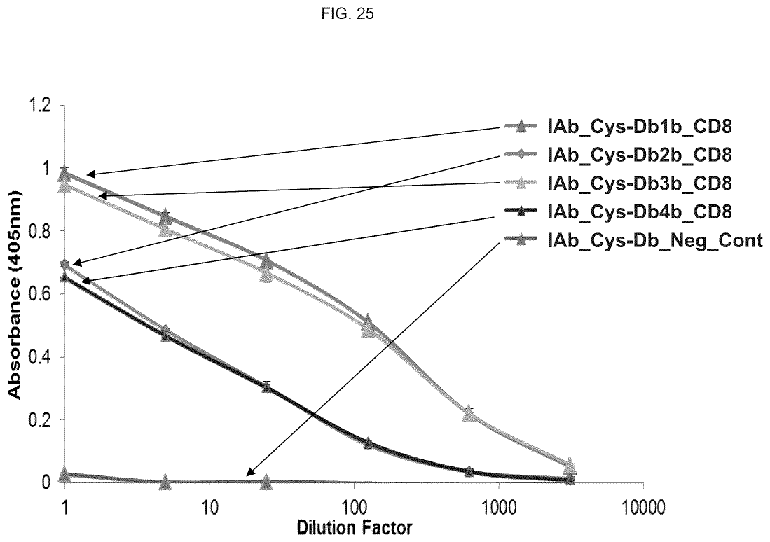

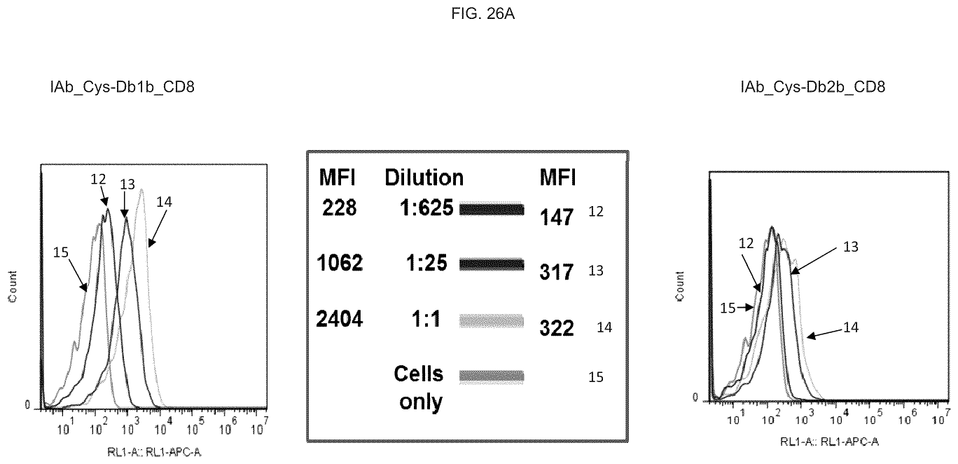

All four variants exhibited concentration-dependent binding to the rhCD8 protein (FIG. 25). The transfection supernatant from a negative control cys-diabody that was included in the analysis did not show any binding to CD8 (FIG. 25).

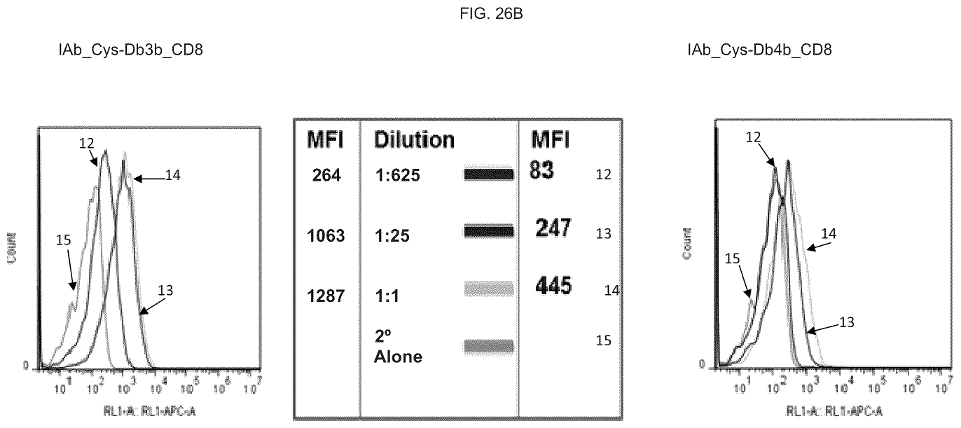

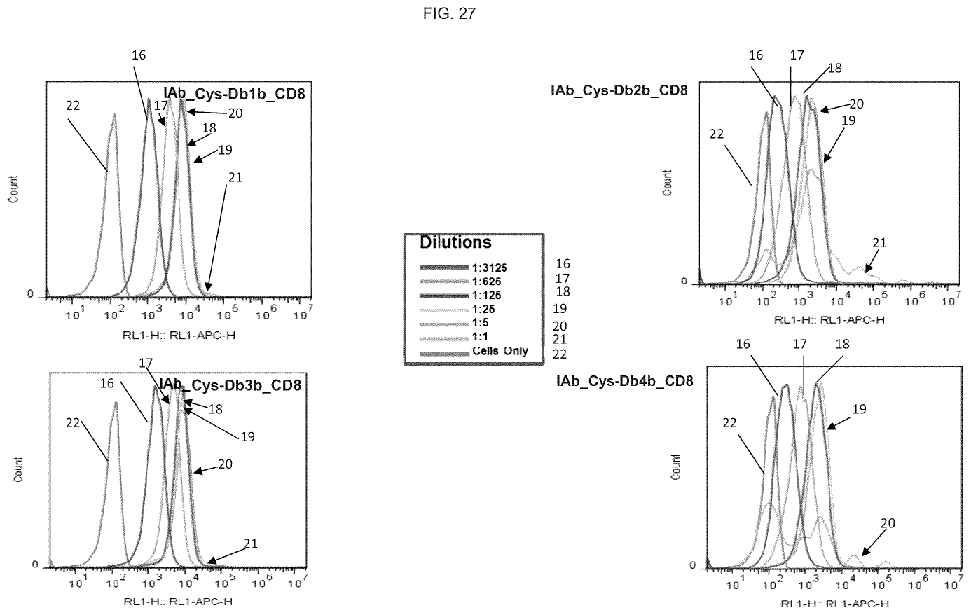

All four IAb_Cys-Dbb_CD8 variants were further tested for binding to cellular human CDS using flow cytometry. Supernatants from the transient transfection were tested for binding to a PC3-CD8 cells (FIGS. 26A and 26B). The parental OKT8 was included as a positive control for binding (data not shown). The PC3 cells were included as negative control and confirmed that there was no non-specific binding of the cys-diabody variants to this cell (data not shown). IAb_Cys-Db1b_CD8 and IAb_Cys-Db3b_CD8 demonstrated higher Mean Fluorescence Intensity (MFI) compared to the other two variants. All histograms in FIGS. 26A and 26B show APC signal vs. counts. Supernatants from transfection of the variants were evaluated for binding to PC3-CDS cells. Cells were subsequently stained with APC-conjugated anti-His antibody. 1×10⁵ cells/point and 10,000 events were analyzed for each point.

The IAb_Cys-Dbb_CD8 variants were evaluated for their ability to bind endogenous CD8 expressing HPB-ALL cells. All IAb_Cys-Db 1b and 3b variants bound to the HPB-ALL cells (FIG. 27). The parental OKT8 was included as a positive control for binding, and the PC3 cells were included as negative cell line. No binding was seen by the cys-diabody variants on PC3 cells that did not express CD8 (data not shown). Supernatants from transfection of the variants were evaluated for binding to endogenous CD8 expressing HPB-ALL cells. Cells were subsequently stained with APC-conjugated anti-His antibody. All histograms show APC signal vs. counts. 1×10⁵ cells/point and 10,000 events were analyzed for each point.

### EXAMPLE 11: IN VIVO DETECTION OF CD8

A humanized CD8 cys-diabody of Table 0.3 is conjugated with a relevant chelator via C-terminal cysteines on the cys-diabody and subsequently radiolabeled with an isotope of In111, (or in the alternative, Zr89 or Cu64). Alternatively, the cys-diabody can be radiolabeled after attaching relevant chelators to Lysine residues or directly radiolabeled with Iodine.

The cys-diabody is infused intravenously into a healthy human subject. The cys-diabody is incubated in the human subject for 10 minutes post-infusion. Within the same day as the incubation, the localization of the cys-diabody is detected via a PET scan or external scintillation system.

Localization of cys-diabody is used to determine localization of CD8 in the subject.

### EXAMPLE 12: IN VIVO DETECTION OF CD8

A minibody from Table 0.2 is conjugated with a relevant chelator via Lysine residues on the minibody and subsequenty radiolabeled with an isotope of In111 (or in the alternative, Zr89 or Cu64). Alternatively, the minibody can be radiolabeled by directly radiolabeling with lodine via Tyrosine residues.

The minibody is infused intravenously into a healthy human subject. The minibody is incubated in the human subject for 10 minutes post-infusion. On the same day as the incubation, the localization of the minibody is detected via a PET scan or external scintillation system.

Localization of cys-diabody is used to determine localization of CD8 in the subject.

### EXAMPLE 13: IN VIVO DETECTION OF CD8

A humanized CD8 minibody that is a homodimer of monomers of SEQ ID NO:12 is provided. The minibody is infused intravenously into a healthy human subject. The minibody is incubated in the human subject for 1 hour post-infusion. A secondary antibody, a humanized cys-diabody that binds specifically to the CD8 minibody and is conjugated to 33P is provided. Within the same day as the incubation, the secondary antibody is infused into to subject. The secondary antibody is incubated for one hour. The localization of the minibody is detected via PET imaging, via a marker on the secondary antibody.

Localization of minibody is used to determine localization of CD8 in the subject.

### EXAMPLE 14: THERAPEUTIC TREATMENT USING A CYS-DIABODY

A CD8 cys-diabody that is a homodimer of monomers of Table 0.3 is provided. The cys-diabody is infused intravenously into a subject having a CD8 related disorder in an amount adequate to bind to sufficient levels of CD8 in the subject to provide a lessening of the symptoms of the CD8 related disorder. The cys-diabody is conjugated to Yttrium 90.

### EXAMPLE 15: THERAPEUTIC TREATMENT USING A MINIBODY

A CD8 minibody of Table 0.2 is provided. The minibody is injected into a patient who has been vaccinated with an antigen to an infectious disease or with a tumor associated antigen. The CD8 directed fragments augment the immune response and enhance the cytolytic activity of CD8 expressing T cells.

### EXAMPLE 16: THERAPEUTIC TREATMENT USING A CYS-DIABODY

A CD8 cys-diabody that is a homodimer of a monomer of Table 0.3 is provided. The cys-diabody is infused intravenously into a subject having a CD8 related disorder in an amount adequate to bind to sufficient levels of CD8 in the subject to provide a lessening of the symptoms of the CD8 related disorder. The cys-diabody is conjugated to Lu177Tx. The CD8 cys-diabody binds to a cell expressing CD8 and the Lu177Tx results in the killing of the cell.

In this application, the use of the singular can include the plural unless specifically stated otherwise or unless, as will be understood by one of skill in the art in light of the present disclosure, the singular is the only functional embodiment. Thus, for example, “a” can mean more than one, and “one embodiment” can mean that the description applies to multiple embodiments.

### Incorporation By Reference

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application; including but not limited to defined terms, term usage, described techniques, or the like, this application controls.

### Equivalents

The foregoing description and Examples detail certain embodiments. It will be appreciated, however, that no matter how detailed the foregoing may appear in text, the invention may be practiced in many ways and the invention should be construed in accordance with the appended claims and any equivalents thereof.

1. (canceled)

2. An isolated nucleic acid encoding one or more polypeptides of an antigen binding construct comprising:
a heavy chain complementarity determining region 1 (HCDR1) of SEQ ID NO:3 or 6;
a HCDR2 of SEQ ID NO:3 or 6;
a HCDR3 of SEQ ID NO:3 or 6;
a light chain complementarity determining region 1 (LCDR1) of SEQ ID NO:9;
a LCDR2 of SEQ ID NO:9; and
a LCDR3 of SEQ ID NO:9.

3. The isolated nucleic acid of claim 2, wherein the antigen binding construct binds specifically to CD8.

4. The isolated nucleic acid of claim 2, wherein the antigen binding construct is bispecific.

5. The isolated nucleic acid of claim 2, wherein the antigen binding construct is a monovalent scFv.

6. The isolated nucleic acid of claim 2, wherein the antigen binding construct comprises a HFR3 of a HFR3 of SEQ ID NO:48.

7. The isolated nucleic acid of claim 2, wherein the antigen binding construct comprises a LFR1 of a LFR1 of SEQ ID NO:42.

8. The isolated nucleic acid of claim 2, wherein the antigen binding construct is a minibody.

9. The isolated nucleic acid of claim 6, wherein the one or more polypeptides comprises a polypeptide that comprises, from N- to C-terminus:
a single-chain variable fragment (scFv) that binds to CD8, the scFv comprising:
a variable heavy (V_{H}) domain linked to a variable light (V_{L}) domain, the V_{H} domain comprising the HCDR1, HCDR2, and the HCDR3, and the V_{L} domain comprising the LCDR1, LCDR2, and the LCDR3; and
a hinge-extension domain comprising a human IgG1 hinge region; and
a human IgG C_{H}3 sequence.

10. The isolated nucleic acid of claim 2, wherein the antigen binding construct is a cys-diabody.

11. The isolated nucleic acid of claim 10, wherein the one or more polypeptides comprises a polypeptide that comprises:
a single-chain variable fragment (scFv) that binds to CD8, the scFv comprising:
a variable heavy (V_{H}) domain linked to a variable light (V_{L}) domain, the V_{H} domain comprising the HCDR1, HCDR2, and the HCDR3, and the V_{L} domain comprising the LCDR1, LCDR2, and the LCDR3; and
a C-terminal cysteine.

12. The isolated nucleic acid of claim 11, wherein the order of the variable domains, from N terminus to C terminus of the polypeptide is V_{L}, V_{H}.

13. The isolated nucleic acid of claim 11, wherein the order of the variable domains, from N terminus to C terminus of the polypeptide is V_{H}, V_{L}.

14. A cell line producing an antigen binding construct comprising:
a HCDR1 of SEQ ID NO:3 or 6;
a HCDR2 of SEQ ID NO:3 or 6;
a HCDR3 of SEQ ID NO:3 or 6;
a LCDR1 of SEQ ID NO:9;
a LCDR2 of SEQ ID NO:9; and
a LCDR3 of SEQ ID NO:9.

15. The cell line of claim 14, wherein the antigen binding construct is a minibody.

16. The cell line of claim 14, wherein the antigen binding construct is a cys-diabody.

17. A method of targeting a therapeutic agent to a CD8, comprising administering to a subject an antigen binding construct comprising:
a HCDR1 of SEQ ID NO:3 or 6;
a HCDR2 of SEQ ID NO:3 or 6;
a HCDR3 of SEQ ID NO:3 or 6;
a LCDR1 of SEQ ID NO:9;
a LCDR2 of SEQ ID NO:9; and
a LCDR3 of SEQ ID NO:9,
wherein the antigen binding construct is conjugated to a therapeutic agent.

18. The method of claim 17, wherein the antigen binding construct is a minibody.

19. The method of claim 17, wherein the antigen binding construct is a cys-diabody

20. The method of claim 17, wherein the therapeutic agent comprises a chemotherapeutic agent.
